(19) United States
(12) Patent Application Publication
(10) Pub. No.: US 2004/0053900 A1
(43) Pub. Date: Mar. 18, 2004

(54) Method of using a COX-2 inhibitor and an aromatase inhibitor as a combination therapy

(75) Inventor: Jaime L. Masferrer, Ballwin, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Pharmacia Corporation

(21) Appl. No.: 10/421,685

(22) Filed: Apr. 23, 2003

Related U.S. Application Data
(63) Continuation-in-part of application No. 09/470,951, filed on Dec. 22, 1999, now abandoned.
(60) Provisional application No. 60/113,786, filed on Dec. 23, 1998.

(51) Int. Cl. A61K 31/56; A61K 31/415; A61K 31/365

(52) U.S. Cl. 514/171; 514/406; 514/473

(57) ABSTRACT

The present invention provides compositions and methods to treat, prevent or inhibit a neoplasia, a neoplasia-related disorder or osteoporosis in a mammal using a combination of a COX-2 inhibitor and an aromatase inhibitor.

This application is a continuation-in-part of U.S. patent application Ser. No. 09/470,951, filed Dec. 22, 1999, which claims priority to United States provisional patent application Serial No. 60/113,786, filed Dec. 23, 1998. The text of these applications is hereby incorporated by reference.

### FIELD OF THE INVENTION

The present invention relates to compositions and methods for the treatment, prevention or inhibition of a neoplasia, a neoplasia-related disorder or osteoporosis in a mammal using a combination of a COX-2 selective inhibitor and an aromatase inhibitor.

### BACKGROUND OF THE INVENTION

Cancer is now the second leading cause of death in the United States and over 8,000,000 persons in the United States have been diagnosed with cancer. In 1995, cancer accounted for 23.3% of all deaths in the United States. (See U.S. Dept. of Health and Human Services, National Center for Health Statistics, Health United States 1996-97 and Injury Chartbook 117 (1997)).

Cancer is not fully understood on the molecular level. It is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called proto-oncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A neoplasm, or tumor, is an abnormal, unregulated, and disorganized proliferation of cell growth, and is generally referred to as cancer. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now primarily treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer.

The adverse effects of systemic chemotherapy used in the treatment of neoplastic disease are most feared by patients undergoing treatment for cancer. Of these adverse effects nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications (see M. D. Abeloff, et al., Alopecia and Cutaneous Complications, p. 755-56 in Abeloff, M. D., Armitage, J. O., Lichter, A. S., and Niederhuber, J. E. (eds.), Clinical Oncology, Churchill Livingston, N.Y., 1992, for cutaneous reactions to chemotherapy agents), such as pruritis, urticaria, and angioedema; neurological complications; pulmonary and cardiac complications in patients receiving radiation or chemotherapy; and reproductive and endocrine complications. Chemotherapy-induced side effects significantly impact the quality of life of the patient and may dramatically influence patient compliance with treatment.

Additionally, adverse side effects associated with chemotherapeutic agents are generally the major dose-limiting toxicity (DLT) in the administration of these drugs. For example, mucositis, is one of the major dose limiting toxicities for several anticancer agents, including the antimetabolite cytotoxic agents 5-FU, methotrexate, and antitumor antibiotics, such as doxorubicin. Many of these chemotherapy-induced side effects if severe, may lead to hospitalization, or require treatment with analgesics for the treatment of pain.

Prostaglandins are arachidonate metabolites that are produced in virtually all mammalian tissues and possess diverse biologic capabilities, including vasoconstriction, vasodilation, stimulation or inhibition of platelet aggregation, and immunomodulation, primarily immunosuppression. They are implicated in the promotion of development and growth of malignant tumors (Honn et al., Prostaglandins, 21, 833-64 (1981); Furuta et al., Cancer Res., 48, 3002-7 (1988); Taketo, J. Natl. Cancer Inst., 90, 1609-20 (1998)). They are also involved in the response of tumor and normal tissues to cytotoxic agents such as ionizing radiation (Milas and Hanson, Eur. J. Cancer, 31A, 1580-5 (1995)). Prostaglandin production is mediated by two cyclooxygenase enzymes, COX-1 and COX-2. Cyclooxygenase-1 (COX-1) is constitutively expressed and is ubiquitous. Cyclooxygenase-2 (COX-2) is induced by diverse inflammatory stimuli (Isakson et al., Adv. Pros. Throm. Leuk Res., 23, 49-54 (1995)).

Traditional nonsteroidal anti-inflammatory drugs (NSAIDs) non-selectively inhibit both cyclooxygenase enzymes and consequently can prevent, inhibit, or abolish the effects of prostaglandins. Increasing evidence shows that NSAIDs can inhibit the development of cancer in both experimental animals and in humans, can reduce the size of established tumors, and can increase the efficacy of cytotoxic cancer chemotherapeutic agents.

Investigations have demonstrated that indomethacin prolongs tumor growth delay and increases the tumor cure rate in mice after radiotherapy (Milas et al., Cancer Res., 50, 4473-7 (1990)). The influence of oxyphenylbutazone and radiation therapy on cervical cancer has been studied (Weppelmann and Monkemeier, Gyn. Onc., 17(2), 196-9 (1984)). However, treatment with NSAIDs is limited by toxicity to normal tissue, particularly by ulcerations and bleeding in the gastrointestinal tract, ascribed to the inhibition of COX-1. Recently developed selective COX-2 inhibitors exert potent anti-inflammatory activity but cause fewer side effects.

COX-2 has been linked to all stages of carcinogenesis (S. Gately, Cancer Metastasis Rev., 19(1/2), 19-27 (2000)). Recent studies have shown that compounds which preferentially inhibit COX-2 relative to COX-1 restore apoptosis and inhibit cancer cell proliferation (E. Fosslien, Crit. Rev. Clin. Lab. Sci., 37(5), 431-502 (2000)). COX-2 inhibitors, such as celecoxib, are showing promise for the treatment and prevention of colon cancer (R. A. Gupta et al., Ann. N.Y. Acad. Sci., 910, 196-206 (2000)) and in animal models for the treatment and prevention of breast cancer (L. R. Howe et al., Endocr.-Relat. Cancer, 8(2), 97-114 (2001)).

COX-2 inhibitors have been described for the treatment of cancer (WO 98/16227) and for the treatment of tumors (EP 927,555). Celecoxib, an anti-inflammatory drug showing a high degree of selectivity for COX-2, exerted potent inhibition of fibroblast growth factor-induced corneal angiogenesis in rats (Masferrer et al., Proc. Am. Assoc. Cancer Research, 40, 396 (1999)).

In 1896 Cecil Beatson demonstrated that ovariectomy resulted in tumor regression in premenopausal breast cancer patients. Subsequently, estrogens were identified as the mediator of ovarian dependency. The biological effect of estrogens was found to be mediated by the stimulation of a nuclear estrogen receptor (ER), which belongs to a family of hormone-activated transcription factors that can initiate or enhance the transcription of genes containing specific hormone response elements. Further, the sensitivity of breast cancer to estrogens has been found to increase in tumors positive for ER. Over the last two decades, several approaches have been attempted to develop pharmacological agents able to reduce estrogen effect.

Two pharmacological approaches are currently available: 1) the antiestrogens, which antagonize the effect of estrogens at the ER level; 2) the aromatase (estrogen synthetase) inhibitors, which inhibit the estrogen production, i.e., the conversion of the substrates androstenedione and testosterone to estrone and estradiol, respectively. The prototype antiestrogen, tamoxifen, is now largely used in the adjuvant systemic therapy of localized breast cancer (i.e., systemic therapy given at the time of primary local treatment in the absence of demonstrated metastasis) and in the treatment of advanced (metastatic) breast cancer. However, resistance to tamoxifen occurs, due to: 1) the intrinsic estrogenic effect of tamoxifen (i.e., partial estrogen agonism); 2) the formation of tamoxifen's estrogenic metabolites; 3) the stimulation by tamoxifen and its metabolites of a mutated ER; 4) the growth of estrogen independent tumor cells. In addition, some concerns are now being considered in the use of tamoxifen in the early disease, due to the increased risk of endometrial cancer. Therefore, new hormonal therapies without the negative effects of either tamoxifen or other similar compounds are under extensive evaluation.

The aromatase inhibitors represent one such new antihormonal treatment for breast cancer (V. C. O. Njar et al., Drugs, 58(2), 233-255 (1999)). In premenopausal women, the ovarian aromatase is the main source of circulating estrogens. In postmenopausal women, adipose tissue is considered to be the main site for estrogen synthesis. In addition, aromatase activity has been shown in the breast tissue, including the tumor itself. Therefore, the very high levels of intratumoral estrogens in comparison to the circulating estrogens are due to the local estrogen synthesis through the aromatase enzyme. Various steroidal and non-steroidal compounds have been described as aromatase inhibitors, including the steroidal derivatives exemestane and formestane, and the nonsteroidal derivatives aminoglutethimide, vorozole, fadrozole, letrozole, anastrozole and YM-511 (Kudoh, M. et al., J. Steroid. Biochem. Molec. Biol., 58,189-194 (1996)). The use of exemestane in postmenopausal women with advanced breast cancer has been reviewed (D. Clemett et al., Drugs, 59(6), 1279-1296 (2000)). Many clinical trials have shown that these compounds represent an effective second line treatment for metastatic breast cancer refractory to tamoxifen. In addition, these compounds are being clinically evaluated in the adjuvant setting, either alone or combined with tamoxifen, and as first-line treatment of the metastatic disease. The more complete estrogen blockade via aromatase inhibition is expected to result in greater tumor response than with tamoxifen, due to the weak or partial estrogen agonist effect of tamoxifen as above discussed.

Breast cancer was one of the first solid tumors to be treated with chemotherapy involving cytotoxic agents, and one of the first tumors to be treated with polychemotherapy. Menopausal status and ER status play an important role in therapy selection either in early or metastatic breast cancer. Chemotherapy is more commonly used in premenopausal women who are more likely to have ER-negative tumors. In the advanced disease, chemotherapy is recommended for ER-negative tumors and after hormonotherapy failures for ER-positive tumors. In several randomized trials, polychemotherapy has been established to be superior to monochemotherapy either in the adjuvant or metastatic setting. The cytotoxic compounds generally used in the polychemotherapy of breast cancer or that are under clinical evaluation belong to various classes including: 1) topoisomerase II inhibitors, such as the antracyclines doxorubicin, epirubicin, idarubicin and nemorubicin, the anthraquinones mitoxantrone and losoxantrone, and the podophillotoxines etoposide and teniposide; 2) antimicrotubule agents, such as the taxanes paclitaxel and docetaxel, and the vinkaalkaloids vinblastine and vinorelbine; 3) alkylating agents, such as cyclophosphamide, ifosfamide and melphalan and the alkycycline derivative PNU-159548 (C. Geroni et al., Proc. Am. Assoc. Cancer Res. 39, 223 (1998)); 4) antineoplastic antimetabolites, such as 5-fluorouracil, capecitabine, gemcitabine, methotrexate and edatrexate; 5) topoisomerase I inhibitors, such as topotecan, irinotecan, 9-nitrocamptothecin and the macromolecular camptothecin conjugate PNU-166148 (compound A1 in WO 99/17804).

Despite intensive efforts directed at prevention and early diagnosis, breast cancer remains one of the leading causes of morbidity and mortality in women. Although early-stage disease is now frequently cured by surgical intervention and adjuvant hormonal and/or chemotherapy, the prognosis for women with advanced or with metastatic disease remains poor. In fact, a median survival of only 2-3 years has been consistently reported over the last 20 years, in spite of the introduction of novel agents. Therefore, in advanced breast cancer patients, palliation of symptoms remains one of the primary objectives of treatment, and maintaining a reasonable quality of life is of paramount importance. Hormonal therapy is often the treatment of choice in such patients. However, current hormonal treatments of breast cancer in patients not selected on the basis of their receptor status, gives a maximal response rate of 30-35%. The median duration of response is 1 to 2 years and is influenced by the site of disease. If a patient's cancer responds to hormonal therapy but later progresses, the cancer may respond again to a second hormonal therapy, but the response rate decreases and the duration of response becomes shorter. Eventually, nearly all breast cancers become refractory to hormonal manipulation and the patients are candidates for cytotoxic chemotherapy. Chemotherapy is more toxic than hormonal therapy and is therefore generally reserved for patients refractory to hormonal treatment, patients with extensive visceral involvement, or patients with a rapidly growing tumor. Combination chemotherapy is generally more effective than single agent treatment. However, only 15% of patients have a complete remission, the duration of the response is limited, all the tumors become resistant to chemotherapy and the patients die. Therefore a major goal in breast cancer therapy is to develop new treatment modalities in order to increase tumor response and survival.

Accordingly, it would be desirable to have a drug combination modality having improved action over currently used treatment modalities. Ideally such a combination would have increased efficacy, e.g. by providing both better control of breast tumor growth and a longer duration of action. Such a strategy would also result in less toxic side effects, thus allowing for the administration of lower dosage levels of the chemotherapeutic agents. Adverse side effects induced by anticancer therapy have become of major importance to the clinical management of cancer patients undergoing treatment for cancer or neoplasia disease.

Recent studies have shown that there is a strong linear correlation between aromatase and cyclooxygenase gene expression in human breast cancer specimens (R. W. Brueggemeier, et al., Cancer Letters 140, 27-35 (1999)).

WO 98/16227 describes the use of COX-2 inhibitors in the treatment or prevention of neoplasia.

WO 98/41511 describes 5-(4-sulphonylphenyl)-pyridazinone COX-2 inhibitors used for treating cancer.

WO 98/41516 describes (methylsulphonyl)phenyl-2-(5H)-furanone COX-2 inhibitors that can be used in the treatment of cancer.

WO 98/47890 describes substituted benzopyran derivatives that may be used alone or in combination with other active principles for the treatment of neoplasia.

WO 96/41645 describes a combination comprising a COX-2 inhibitor and a leukotriene A hydrolase inhibitor.

WO 97/11701 describes a combination comprising a COX-2 inhibitor and a leukotriene B4 receptor antagonist useful in treating colorectal cancer.

WO 97/29774 describes the combination of a COX-2 inhibitor and prostaglandin or antiulcer agent useful in treating cancer.

WO 97/36497 describes a combination comprising a COX-2 inhibitor and a 5-lipoxygenase inhibitor useful in treating cancer.

WO 99/18960 describes a combination comprising a COX-2 inhibitor and an induced nitric-oxide synthase inhibitor (iNOS) that can be used to treat colorectal and breast cancer.

WO 99/25382 describes compositions containing a COX-2 inhibitor and a N-methyl-d-aspartate (NMDA) antagonist used to treat cancer and other diseases.

Osteoporosis is the most common type of metabolic bone disease and is characterized by the thinning of bone tissue and the progressive loss of bone density. Osteoporosis may occur when the body does not form enough new bone or when too much old bone is reabsorbed by the body. In the aging process, the body may reabsorb calcium and phosphate from the bones, making the bone tissue weaker. This situation results in fragile, brittle bones that are subject to fractures, even in the absence of trauma.

It is estimated that 23 percent of American women over the age of 50 have osteoporosis and an even larger percentage have osteopenia, which is abnormally low bone density. Researchers estimate that 50% of women over 50 will suffer an osteoporosis related fracture at some point in their life.

Therapies for the prevention and treatment of osteoporosis include estrogen replacement therapy and the use of drugs that slow the rate of bone loss, such as calcitonin, alendronate, and raloxifene (Lopez, F. J., Curr. Opin. Chem. Biol., 4(4), 383-393 (2000)).

U.S. Pat. No. 6,271,253 describes substituted benzopyran selective COX-2 inhibitors useful in treating or preventing bone resorption associated with osteoporosis.

WO 01/40216 describes heterocyclo-alkylsulfonyl pyrazole COX-2 inhibitors useful in treating osteoporosis.

U.S. Pat. No. 6,222,048 describes diaryl-2-(5H)-furanone COX-2 inhibitors useful in the prevention of bone loss.

WO 01/116138 describes sulfonylphenylpyrazole compounds useful as COX-2 inhibitors for the treatment of osteoporosis.

U.S. Pat. No. 6,071,936 describes substituted pyridine selective COX-2 inhibitors useful for the treatment of decreasing bone loss, particularly in postmenopausal women.

WO 99/11605 describes certain 5-alkyl-2-arylaminophenylacetic acids and derivatives as selective COX-2 inhibitors useful for the treatment of osteoporosis.

WO 01/03719 describes the use of a novel polypeptide, osteoprotegerin, in combination with a COX-2 inhibitor to treat bone diseases characterized by increased bone loss, such as osteoporosis.

U.S. Pat. No. 6,306,874 describes tyrosine kinase inhibitors, in combination with selective COX-2 inhibitors as being useful to treat and prevent conditions related to bone resorption, such as osteoporosis.

However, new therapies for the treatment and prevention of osteoporosis with minimized side effects are still needed. In particular, novel therapies for the treatment, prevention or inhibition of both neoplasia and osteoporosis, would be desirable.

### SUMMARY OF THE INVENTION

Among its several embodiments, the present invention provides a composition comprising an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention or inhibition of a neoplasia, a neoplasia-related disorder, or osteoporosis.

In another embodiment, the present invention provides a combination therapy method for the treatment, prevention, or inhibition of a neoplasia, a neoplasia-related disorder, or osteoporosis in a mammal in need thereof, comprising administering to the mammal an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention, or inhibition of a neoplasia, a neoplasia-related disorder, or osteoporosis.

In yet another embodiment, the present invention provides a pharmaceutical composition comprising an amount of a COX-2 inhibitor compound source and an amount of an aromatase inhibitor and a pharmaceutically-acceptable excipient.

In still another embodiment, the present invention provides a kit that is suitable for the treatment, prevention of inhibition of a neoplasia or a neoplasia-related disorder or osteoporosis, wherein the kit comprises a first dosage form comprising a COX-2 inhibitor compound source and a second dosage form comprising an aromatase inhibitor, in quantities which comprise a therapeutically effective amount of the compounds for the treatment, prevention or inhibition of a neoplasia, a neoplasia-related disorder, or osteoporosis.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

Definitions

The following definitions are provided in order to aid the reader in understanding the detailed description of the present invention.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. here used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six-carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl and trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

The phrase “combination therapy” (or “co-therapy”) embraces the administration of a COX-2 inhibitor and an aromatase inhibitor as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different antineoplastic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrase “therapeutically effective” is intended to qualify the amount of inhibitors in the therapy. This amount will achieve the goal, e.g., of treating, preventing or inhibiting neoplasia or a neoplasia-related disorder, or of osteoporosis, where that is the therapeutic objective.

“Therapeutic compound” means a compound useful in the treatment, prevention or inhibition of neoplasia or a neoplasia-related disorder, or of osteoporosis, where that is the therapeutic objective.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The term “comprising” means “including the following elements but not excluding others.”

Combinations and Methods

Among its several embodiments, the present invention provides a composition comprising an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention or inhibition of a neoplasia, a neoplasia-related disorder, or osteoporosis.

In one embodiment, the source of the COX-2 inhibitor compound is a COX-2 inhibitor.

In another embodiment, the COX-2 inhibitor is a COX-2 selective inhibitor.

In yet another embodiment, the source of the COX-2 inhibitor compound is a prodrug of a COX-2 inhibitor compound, illustrated herein with parecoxib.

In still another embodiment, the present invention provides a combination therapy method for the treatment, prevention, or inhibition of a neoplasia, a neoplasia-related disorder, or osteoporosis in a mammal in need thereof, comprising administering to the mammal an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention, or inhibition of a neoplasia, a neoplasia-related disorder, or osteoporosis.

In an additional embodiment, the present invention provides a pharmaceutical composition comprising an amount of a COX-2 inhibitor compound source and an amount of an aromatase inhibitor and a pharmaceutically-acceptable excipient.

In yet an additional embodiment, the present invention provides a kit that is suitable for the treatment, prevention of inhibition of a neoplasia or a neoplasia-related disorder or osteoporosis, wherein the kit comprises a first dosage form comprising a COX-2 inhibitor compound source and a second dosage form comprising an aromatase inhibitor, in quantities which comprise a therapeutically effective amount of the compounds for the treatment, prevention or inhibition of a neoplasia, a neoplasia-related disorder, or osteoporosis.

The methods and combinations of the present invention provide one or more benefits. Combinations of COX-2 inhibitors with the compounds, compositions, agents and therapies of the present invention are useful in treating, preventing or inhibiting neoplasia or a neoplasia-related disorder or osteoporosis. Preferably, the COX-2 inhibitors and the compounds, compositions, agents and therapies of the present invention are administered in combination at a low dose, that is, at a dose lower than has been conventionally used in clinical situations.

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Alternatively, the methods and combinations of the present invention can also maximize the therapeutic effect at higher doses.

When administered as a combination, the therapeutic agents can be formulated as separate compositions that are given at the same time or different times, or the therapeutic agents can be given as a single composition.

There are many uses for the present inventive combination. For example, aromatase inhibitors and COX-2 selective inhibiting agents (or prodrugs thereof) are each believed to be effective antineoplastic or antiangiogenic agents. However, patients treated with an aromatase inhibitor experience side effects, such as nausea, vomiting, pain and fatigue. The present inventive combination will allow the subject to be administered an aromatase inhibitor at a therapeutically effective dose yet experience reduced or fewer symptoms of nausea, vomiting, pain and fatigue. A further use and advantage is that the present inventive combination will allow therapeutically effective individual dose levels of the aromatase inhibitor and the COX-2 selective inhibitor that are lower than the dose levels of each inhibitor when administered to the patient as a monotherapy.

Inhibitors of the cyclooxygenase pathway in the metabolism of arachidonic acid used in the treatment, prevention or reduction of the risk of developing neoplasia disease may inhibit enzyme activity through a variety of mechanisms. By way of example, the cyclooxygenase inhibitors used in the methods described herein may block the enzyme activity directly by acting as a substrate for the enzyme. The use of a COX-2 selective inhibiting agent is highly advantageous in that they minimize the gastric side effects that can occur with non-selective non-steroidal antiinflammatory drugs (NSAIDs), especially where prolonged treatment is expected.

Besides being useful for human treatment, the present invention is also useful for veterinary treatment of companion animals, exotic animals and farm animals, including mammals, rodents, and the like. More preferred animals include horses, dogs, and cats.

### Cyclooxygenase-2 Selective Inhibitors

A component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

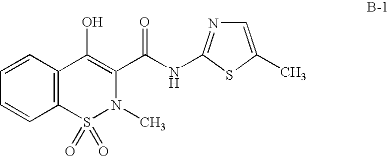

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

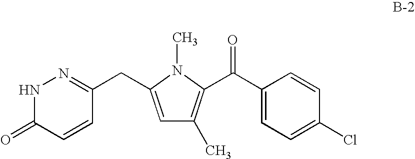

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 1, including the diastereomers, enantiomers, racemates, tautomers, salts, esters, amides and prodrugs thereof.

Benzopyrans that can serve as a cyclooxygenase-2 selective inhibitor of the present invention include substituted benzopyran derivatives that are described in U.S. Pat. No. 6,271,253. One such class of compounds is defined by the general formula shown below in formulas I:

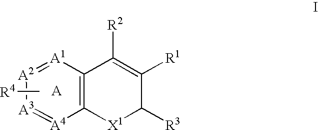

wherein X¹ is selected from O, S, CR^{c}R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl;

wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁C₆-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl;

or an isomer or pharmaceutically acceptable salt thereof.

Another class of benzopyran derivatives that can serve as the Cox-2 selective inhibitor of the present invention includes a compound having the structure of formula II:

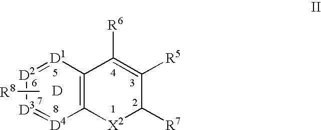

wherein X² is selected from O, S, CR^{c}R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;

or wherein CR^{c}R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl;

wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂ O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

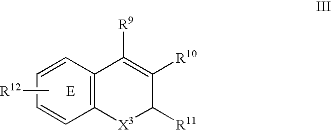

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl;

wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as cyclooxygenase-2 selective inhibitors in the present invention is described by Formulas IV and V:

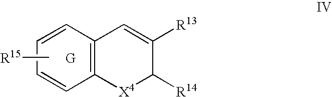

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁵ together with ring G forms a naphthyl radical;

or an isomer or pharmaceutically acceptable salt thereof.

Formula V is:

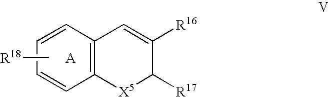

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro-, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The cyclooxygenase-2 selective inhibitor of the present invention can also be a compound having the structure of Formula VI:

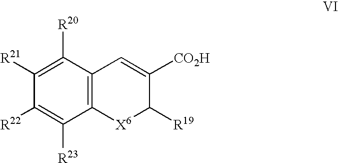

wherein:

X⁶ is selected from the group consisting of O and S;

R¹⁹ is lower haloalkyl;

R²⁰ is selected from the group consisting of hydrido and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl; or an isomer or prodrug thereof.

TABLE 1
Examples of Chromene Cox-2 Selective Inhibitors

| Compound | |
|---|---|
| Number | Structural Formula |
| B-3 | 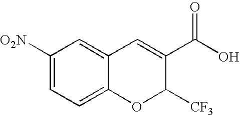 |
| | 6-Nitro-2-trifluoromethyl-2H-1- |
| | benzopyran-3-carboxylic acid |
| B-4 | 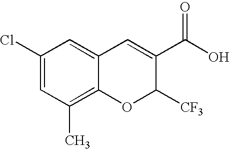 |
| | 6-Chloro-8-methyl-2-trifluoromethyl- |
| | 2H-1-benzopyran-3-carboxylic acid |
| B-5 | 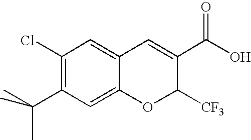 |
| | ((S)-6-Chloro-7-(1,1-dimethylethyl)-2-(trifluoro- |
| | methyl-2H-1-benzopyran-3-carboxylic acid |
| B-6 | 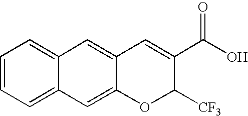 |
| | 2-Trifluoromethyl-2H-naphtho [2, 3-b] |
| | pyran-3-carboxylic acid |
| B-7 | 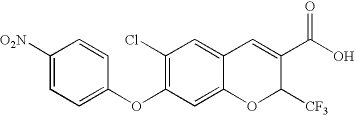 |
| | 6-Chloro-7-(4-nitrophenoxy)-2-(trifluoromethyl)-2H-1- |
| | benzopyran-3-carboxylic acid |
| B-8 | 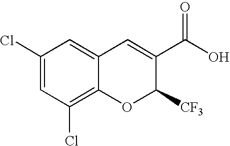 |
| | ((S)-6,8-Dichloro-2-(trifluoromethyl)- |
| | 2H-1-benzopyran-3-carboxylic acid |
| B-9 | 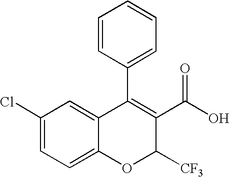 |
| | 6-Chloro-2-(trifluoromethyl)-4-phenyl-2H- |
| | 1-benzopyran-3-carboxylic acid |
| B-10 | 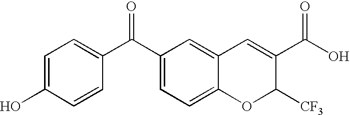 |
| | 6-(4-Hydroxybenzoyl)-2-(trifluoromethyl)- |
| | 2H-1-benzopyran-3-carboxylic acid |
| B-11 | 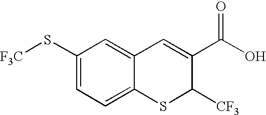 |
| | 2-(Trifluoromethyl)-6-[(trifluoromethyl) thio]- |
| | 2H-1-benzothiopyran-3-carboxylic acid |
| B-12 | 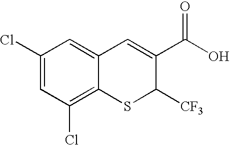 |
| | 6,8-Dichloro-2-trifluoromethyl-2H-1- |
| | benzothiopyran-3-carboxylic acid |
| B-13 | 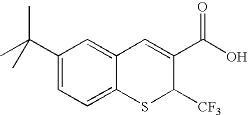 |
| | 6-(1,1-Dimethylethyl)-2-(trifluoromethyl)- |
| | 2H-1-benzothiopyran-3-carboxylic acid |
| B-14 | 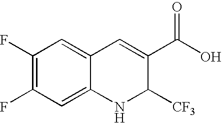 |
| | 6,7-Difluoro-1,2-dihydro-2-(trifluoro- |
| | methyl)-3-quinolinecarboxylic acid |
| B-15 | 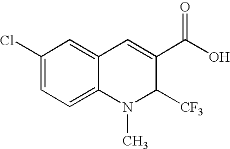 |
| | 6-Chloro-1,2-dihydro-1-methyl-2-(trifluoro- |
| | methyl)-3-quinolinecarboxylic acid |
| B-16 | 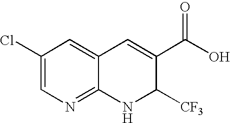 |
| | 6-Chloro-2-(trifluoromethyl)-1,2-dihydro |
| | [1,8]naphthyridine-3-carboxylic acid |
| B-17 | 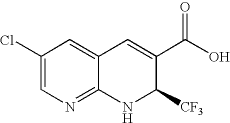 |
| | ((S)-6-Chloro-1,2-dihydro-2-(trifluoro- |
| | methyl)-3-quinolinecarboxylic acid |

Examples of specific compounds that are useful for the cyclooxygenase-2 selective inhibitor include (without limitation):

a1) 8-acetyl-3-(4-fluorophenyl)-2-(4-methylsulfonyl)phenyl-imidazo[1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(d ifluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(d ifluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonyl phenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-d ifluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methyl phenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid;

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of formula VII:

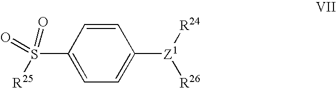

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula VII is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-18 | 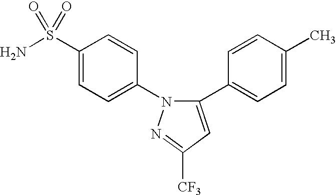 |
| B-19 | 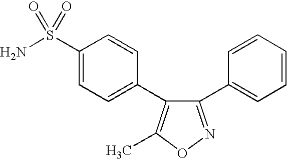 |
| B-20 | 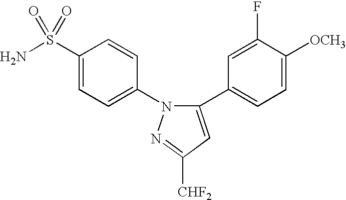 |
| B-21 | 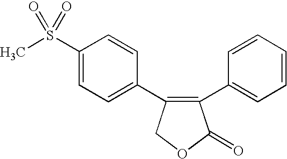 |
| B-22 | 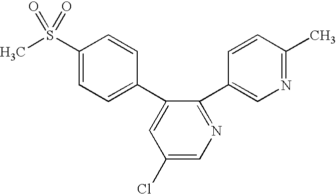 |
| B-23 | 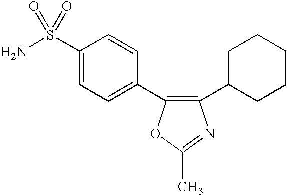 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-19, (See, e.g., U.S. Pat. No. 5,633,272), may be advantageously employed as a source of a cyclooxygenase inhibitor.

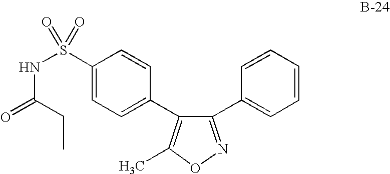

A preferred form of parecoxib is sodium parecoxib.

In another embodiment of the invention, the compound ABT-963 having the formula B-25 that has been previously described in International Publication number WO 00/24719, is another tricyclic cyclooxygenase-2 selective inhibitor which may be advantageously employed.

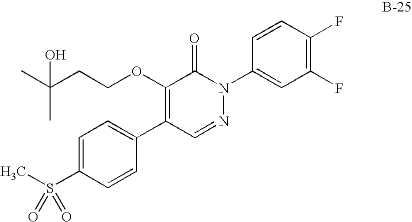

In a yet further embodiment of the invention, the cyclooxygenase inhibitor used in connection with the methods of the present invention can be selected from the class of phenylacetic acid derivative cyclooxygenase-2 selective inhibitors represented by the general structure of Formula VIII:

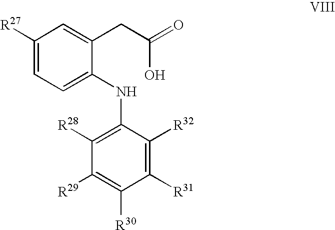

or an isomer, a pharmaceutically acceptable salt, ester, or prodrug thereof; wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl,

provided that R²⁸, R²⁹, R³⁰ and R³¹ are not all fluoro when R²⁷ is ethyl and R³⁰ is H.

A phenylacetic acid derivative cyclooxygenase-2 selective inhibitor that is described in WO 99/11605 is a compound that has the structure shown in Formula VII,

wherein:

R²⁷ is ethyl;

R²⁸ and R³⁰ are chloro;

R²⁹ and R³¹ are hydrogen; and

R³² is methyl.

Another phenylacetic acid derivative cyclooxygenase-2 selective inhibitor is a compound that has the structure shown in Formula VIII,

wherein:

R²⁷ is propyl;

R²⁸ and R³⁰ are chloro;

R²⁹ and R³¹ are methyl; and

R³² is ethyl.

Another phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula VIII,

wherein:

R²⁷ is methyl;

R²⁸ is fluoro;

R³² is chloro; and

R²⁹, R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other cyclooxygenase-2 selective inhibitors that can be used in the present invention have the general structure shown in formula IX, where the J group is a carbocycle or a heterocycle. Preferred embodiments have the structure:

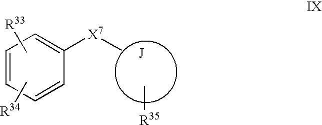

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6-NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N—SO₂CH₃ Na⁺,

(L-745337); and

X is S; J is thiophen-2-yl; R³³ is 4-F; there is no R³⁴ group; and R³⁵ is 5-NHSO₂CH₃, (RWJ-63556); and

X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (06/06/2001); and Iwata, K. et al., in Jpn. J. Pharmacol., 75(2):191-194 (1997).

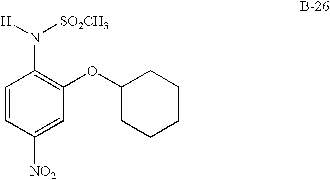

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651. Such diarylmethylidenefuran derivatives have the general formula shown below in formula X:

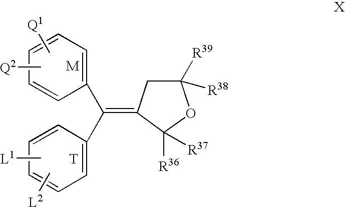

wherein:

rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q¹, Q², L¹ or L² is:

an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R³⁶, R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R³⁶, R³⁷ or R³⁸, R³⁹ are an oxygen atom, or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, 10/04/2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covalently attached to one or more linkers, as described in U.S. Pat. No. 6,395,724.

Compounds that may act as cyclooxygenase-2 inhibitors include conjugated linoleic acid that is described in U.S. Pat. No. 6,077,868.

Materials that can serve as a cyclooxygenase-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

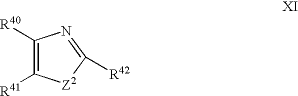

wherein:

Z² is an oxygen atom;

one of R⁴⁰ and R⁴¹ is a group of the formula

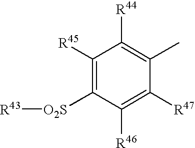

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino,

provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl, and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

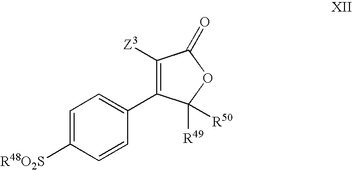

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7)—CO₂H;

R⁴⁸ is selected from the group consisting of NH₂ and CH₃,

R⁴⁹ is selected from the group consisting of:

C₁₋₆ alkyl unsubstituted or substituted with C₃₋₆ cycloalkyl, and

C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as cyclooxygenase-2 selective inhibitors include pyridines that are described in U.S. Pat. Nos. 6, 369,275, 6,127,545, 6,130,334, 6,204,387, 6,071,936, 6,001,843 and 6,040,450, and which have the general formula described by formula XIII:

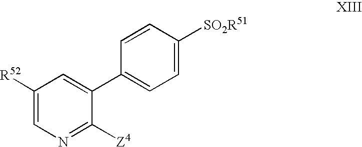

wherein:

R⁵¹ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof),

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j) —C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵, R⁵⁶, R⁵⁷, R⁵⁸, R⁵⁹, R⁶⁰, R⁶¹, R⁶², R⁶³, are each independently chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the cyclooxygenase-2 selective inhibitor of the present invention include diarylbenzopyran derivatives that are described in U.S. Pat. No. 6,340,694. Such diarylbenzopyran derivatives have the general formula shown below in formula XIV:

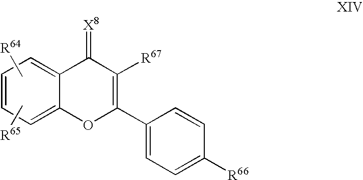

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹ R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

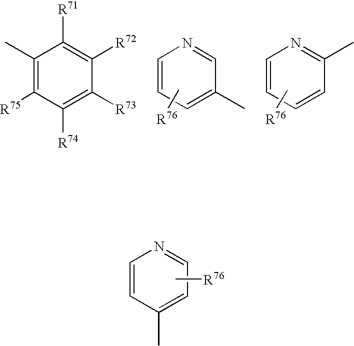

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)ₙR⁶⁸, a group of a formula: NR⁶⁹R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the cyclooxygenase-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

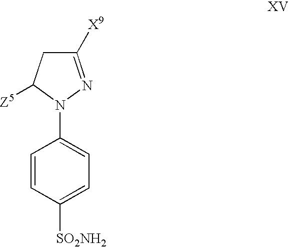

wherein:

X⁹ is selected from the group consisting of C₁-C₆ trihalomethyl, preferably trifluoromethyl; C₁-C₆ alkyl; and an optionally substituted or di-substituted phenyl group of formula XVI:

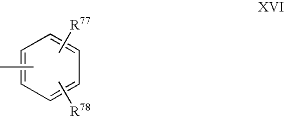

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the cyclooxygenase-2 selective inhibitor of the present invention include heterocycles that are described in U.S. Pat. No. 6,153,787. Such heterocycles have the general formulas shown below in formulas XVII and XVIII:

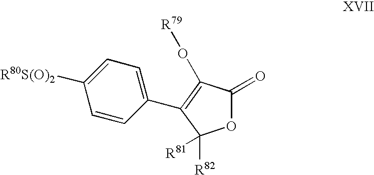

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e) ═O,

(f) dioxolane,

(g) CN; and

R⁸⁰ is selected from the group consisting of:

(a) CH₃,

(b) NH₂,

(c) NHC(O)CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

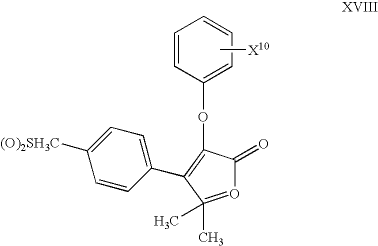

X¹⁰ is fluoro or chloro.

Materials that can serve as the cyclooxygenase-2 selective inhibitor of the present invention include 2,3,5-trisubstituted pyridines that are described in U.S. Pat. No. 6,046,217. Such pyridines have the general formula shown below in formula XIX:

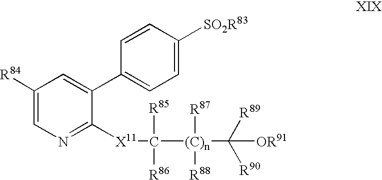

or a pharmaceutically acceptable salt thereof,

wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂R⁹²,

(i) hydroxy,

(j) —C(R⁹³)(R⁹⁴)—OH,

(k) —C₁₋₆ alkyl-CO₂—R⁹⁵,

(l) C₁₋₁₆ fluoroalkoxy,

(m) NO₂,

(n) NR⁹⁶R⁹⁷,

(o) NHCOR⁹⁸;

R⁸⁵ to R⁹⁸ are independently chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is O.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the cyclooxygenase-2 selective inhibitor of the present invention include diaryl bicyclic heterocycles that are described in U.S. Pat. No. 6,329,421. Such diaryl bicyclic heterocycles have the general formula shown below in formula XX:

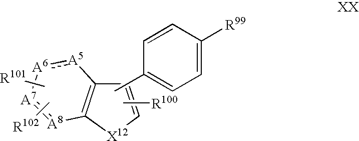

and pharmaceutically acceptable salts thereof wherein:

-A⁵=A⁶-A⁷=A⁸- is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)CH₂—, —C(O)—CH₂—CH₂

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—, —C(O)—O-CH₂—CH₂—,

(f) —C(R¹⁰⁵)₂—O—C(O)—, —C(OO—C(R¹⁰⁵)₂—, —O—C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of:

(a) S(O)₂ CH₃,

(b) S(O)₂ NH₂,

(c) S(O)₂ NHCOCF₃,

(d) S(O)(NH)CH₃,

(e) S(O)(NH)NH₂,

(f) S(O)(NH)NHCOCF₃,

(g) P(O)(CH₃)OH, and

(h) P(O)(CH₃)NH₂;

R¹⁰⁰ is selected from the group consisting of:

(a) C₁₋₆ alkyl,

(b) C₃₋₇, cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹⁰³)(R¹⁴⁰)—OH,

(12) —C(R¹⁰³)(R¹⁰⁴)—O—C₁₋₄ alkyl, and

(13) —C₁₋₆ alkyl-CO₂—R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁₋₁₄ alkyl;

(e) benzoheteroaryl which includes the benzo fused analogs of (d);

R¹⁰¹ and R¹⁰² are the substituents residing on any position of -A⁵=A⁶-A⁷=A⁸- and are selected independently from the group consisting of:

(a) hydrogen,

(b) CF₃,

(c) CN,

(d) C₁₋₆ alkyl,

(e) Q³ wherein Q³ is Q⁴, CO₂H, C(R¹⁰³)(R¹⁰⁴)OH,

(f) —O-Q⁴,

(g) —S-Q⁴, and

(h) optionally substituted:

(1) —C₁₋₅ alkyl-Q³,

(2) —O—C₁₋₅ alkyl-Q³,

(3) —S—C₁₋₅ alkyl-Q³,

(4) —C₁₋₃ alkyl-O—C₁₋₃ alkyl-Q³,

(5) —C₁₋₃ alkyl-S—C₁₋₃ alkyl-Q³,

(6) —C₁₋₅ alkyl-O-Q⁴,

(7) —C₁₋₅ alkyl-S-Q⁴,

wherein the substituent resides on the alkyl chain and the substituent is C₁₋₃ alkyl, and Q³ is Q⁴, CO₂H, C(R¹⁰³)(R¹⁰⁴)OH Q⁴ is CO₂—C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl;

X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—;

—N═C(R¹⁰⁷)—.

Compounds that may act as cyclooxygenase-2 inhibitors include salts of 5-amino or a substituted amino 1,2,3-triazole compound that are described in U.S. Pat. No. 6,239,137. The salts are of a class of compounds of formula XXI:

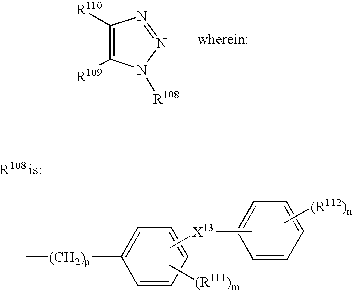

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, lower alkyl, hydroxy, lower alkoxy, amino, lower alkylamino, diloweralkylamino or cyano; and, R¹¹¹ and R¹¹² are independently halogen, cyano, trifluoromethyl, lower alkanoyl, nitro, lower alkyl, lower alkoxy, carboxy, lower carbalkoxy, trifuloromethoxy, acetamido, lower alkylthio, lower alkylsulfinyl, lower alkylsulfonyl, trichlorovinyl, trifluoromethylthio, trifluoromethylsulfinyl, or trifluoromethylsulfonyl; R¹⁰⁹ is amino, mono or diloweralkylamino, acetamido, acetimido, ureido, formamido, formamido or guanidino; and R¹¹⁰ is carbamoyl, cyano, carbazoyl, amidino or N-hydroxycarbamoyl; wherein the lower alkyl, lower alkyl containing, lower alkoxy and lower alkanoyl groups contain from 1 to 3 carbon atoms.

Materials that can serve as a cyclooxygenase-2 selective inhibitor of the present invention include pyrazole derivatives that are described in U.S. Pat. No. 6,136,831. Such pyrazole derivatives have the formula shown below in formula XXII:

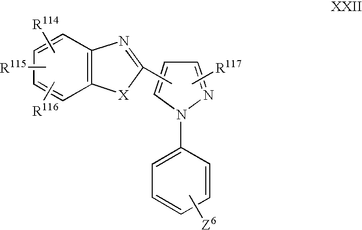

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl; or a pharmaceutically acceptable salt thereof.

Materials that can serve as a cyclooxygenase-2 selective inhibitor of the present invention include substituted derivatives of benzosulphonamides that are described in U.S. Pat. No. 6,297,282. Such benzosulphonamide derivatives have the formula shown below in formula XXIII:

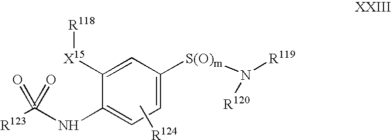

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N— atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;

X¹⁶ denotes halogen, NO₂, —OR¹²¹, —COR¹²¹, —CO₂ R¹²¹, —OCO₂ R¹²¹, —CN, —CONR¹²¹OR¹²², —CONR¹²¹R¹²², —SR¹²¹, —S(O)R¹²¹, —S(O)₂ R¹²¹, NR¹²¹ R¹²², —NHC(O)R¹²¹, —NHS(O)₂ R¹²¹;

n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C— atoms, which can optionally be mono- or polysubstituted by halogen, NO₂, —OR¹²¹, —COR¹²¹, —CO₂R¹²¹,—OCO₂R¹²¹, —CN, —CONR¹²¹OR¹²², —CONR¹²¹R¹²², —SR¹²¹, —S(O)R¹²¹, —S(O)₂ R¹²¹, —NR¹²¹R¹²², —NHC(O)R¹²¹, —NHS(O)₂ R¹²¹, or a polyfluoroalkyl group;

R¹²¹ and R¹²², independently from one another, denote hydrogen, alkyl, aralkyl or aryl; and

m denotes a whole number from 0 to 2;

and the pharmaceutically-acceptable salts thereof.

Materials that can serve as a cyclooxygenase-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

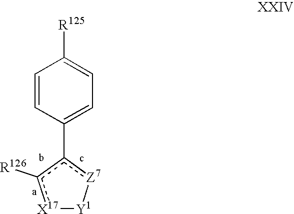

or pharmaceutically acceptable salts thereof wherein:

X¹⁷—Y¹—Z⁷— is selected from the group consisting of:

(a) —CH₂ CH₂ CH₂—,

(b) —C(O)CH₂ CH₂—,

(c) —CH₂ CH₂ C(O)—,

(d) —CR¹²⁹ (R^{129′})—O—C(O)—,

(e) —C(O)—O—CR¹²⁹(R^{129′})—,

(f) —CH₂—NR¹²⁷—CH₂—,

(g) —CR¹²⁹(R^{129′})—NR¹²⁷—C(O)—,

(h) —CR¹²⁸═CR^{128′}′-S—,

(i) —S—CR¹²⁸═CR^{128′}—,

(j) —S—N═CH—,

(k) —CH═N—S—,

(l) —N═CR¹²⁸—O—,

(m) —O—CR¹²⁸═N—,

(n) —N═CR¹²⁸—NH—,

(o) —N═CR¹²⁸—S—, and

(p) —S—CR¹²⁸═N—,

(q) —C(O)—NR¹²⁷—CR¹²⁹(R^{129′})—,

(r) —R¹²⁷ N—CH═CH— provided R₁₂₂ is not —S(O)₂CH₃,

(s) —CH═CH—NR¹²⁷— provided R¹²⁵ is not —S(O)₂CH₃,

when side b is a double bond, and sides a and c are single bonds; and

X¹⁷—Y¹—Z⁷— is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂ CH₃,

(b) S(O)₂ NH₂,

(c) S(O)₂ NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl,

wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹²⁹)(R¹³⁰)—OH,

(12) —C(R¹²⁹)(R¹³⁰)—O—C₁₋₄ alkyl, and

(13) —C₁₋₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxy C₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C₁₋₃ alkyl-S—C₁₋₃ alkyl-Q⁵,

(4) —C₁₋₅ alkyl-O-Q⁵, or

(5) —C₁₋₅ alkyl-S-Q⁵,

wherein the substituent resides on the alkyl and the substituent is C₁₋₃ alkyl;

(h) -Q⁵;

R¹²⁸ and R^{128′} are each independently selected from the group consisting of:

(a) hydrogen,

(b) CF₃,

(c) CN,

(d) C₁₋₆ alkyl,

(e) -Q⁵,

(f) —O-Q⁵;

(g) —S-Q⁵, and

(h) optionally substituted:

(1) —C₁₋₅ alkyl-Q⁵,

(2) —O—C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O-Q⁵,

(7) —C₁₋₅ alkyl-S-Q⁵,

wherein the substituent resides on the alkyl and the substituent is C₁₋₃ alkyl, and

R¹²⁹, R¹²⁹, R¹³⁰, R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂H, CO₂—C₁₋₄ alkyl, tetrazolyl-5-yl, C(R¹³¹)(R¹³²)(OH), or C(R¹³¹)(R¹³²)(O—C₁₋₄ alkyl);

provided that when X—Y-Z is —S—CR¹²⁸=CR^{128′}, then R¹²⁸ and R^{128′} are other than CF₃.

Materials that can serve as a cyclooxygenase-2 selective inhibitor of the present invention include bicycliccarbonyl indole compounds that are described in U.S. Pat. No. 6,303,628. Such bicycliccarbonyl indole compounds have the formula shown below in formula XXV:

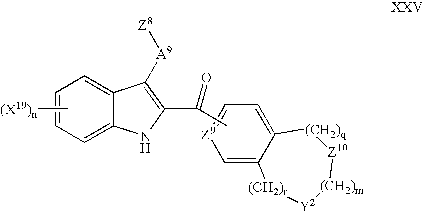

or the pharmaceutically acceptable salts thereof wherein

A⁹ is C₁₋₆ alkylene or —NR¹³³—;

Z⁸ is C(=L³)R¹³⁴, or SO₂R¹³⁵;

Z⁹ is CH or N;

Z¹⁰ and Y² are independently selected from —CH₂—, O, S and —N—R¹³³;

m is 1, 2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;

R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a cyclooxygenase-2 selective inhibitor of the present invention include benzimidazole compounds that are described in U.S. Pat. No. 6,310,079. Such benzimidazole compounds have the formula shown below in formula XXVI:

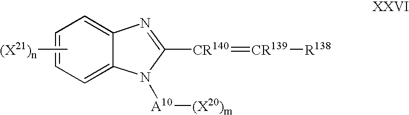

or a pharmaceutically acceptable salt thereof, wherein:

A¹⁰ is heteroaryl selected from

a 5-membered monocyclic aromatic ring having one hetero atom selected from O, S and N and optionally containing one to three N atom(s) in addition to said hetero atom, or

a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₁-C₄ alkanoyl)amonio, N—(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N—(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N,N-di(C₁-C₄ alkyl)amino]sulfonyl;

X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₀-C₄ alkanoyl)amino, N—(C₁-C₄ alkyl)-N—(C₁-C₄ alkanoyl) amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N—(C₁-C₄ alkyl) amino]carbonyl, [N,N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N,N-di(C₁-C₄ alkyl)amino]sulfonyl;

R¹³⁸ is selected from

hydrogen, straight or branched C₁-C₄ alkyl optionally substituted with one to three substituent(s) wherein said substituents are independently selected from halo hydroxy, C₁-C₄ alkoxy, amino, N—(C₁-C₄ alkyl)amino and N,N-di(C₁-C₄ alkyl)amino,

C₃-C₈ cycloalkyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N,N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N,N-di(C₁-C₄ alkyl)amino, phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N,N-di(C₁-C₄ alkyl)amino, [N—(C₁-C₄ alkyl)amino]C₁-C₄ alkyl, [N,N-di(C₁-C₄ alkyl)amino]C₁-C₄ alkyl, N—(C₁-C₄ alkanoyl)amino, N-[C₁-C₄ alkyl)(C₁-C₄ alkanoyl)]amino, N-[(C₁-C₄ alkyl)sulfony]amino, N-[(halo-substituted C₁-C₄ alkyl)sulfonyl]amino, C₁-C₄ alkanoyl, carboxy, (C₁-C₄ alkoxy)carbonyl, carbomoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N-(C₁-C₄ alkyl)amino and N,N-di(C₁-C₄ alkyl)amino,

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Materials that can serve as a cyclooxygenase-2 selective inhibitor of the present invention include indole compounds that are described in U.S. Pat. No. 6,300,363. Such indole compounds have the formula shown below in formula XXVII:

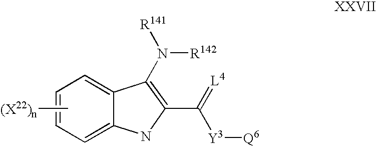

and the pharmaceutically acceptable salts thereof,

wherein:

L⁴ is oxygen or sulfur;

Y³ is a direct bond or C₁₋₄ alkylidene;

Q6 is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from:

(c-1) halo, C₁₋₄ alkyl, halosubstituted C₁₋₄ alkyl, hydroxy, C₁₋₄ alkoxy, halosubstituted C₁₋₄ alkoxy, S(O)ₘR¹⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³, CN, CO₂H, CO₂ (C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo,

C₁₋₄ alkyl, CF₃, hydroxy, OR¹⁴³, S(O)ₘR¹⁴³ amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘ R¹⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³, CN, CO₂ H, CO₂ (C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂ CH₃, SO₂ NH₂, amino, C₁₋₄ alkylamino and NHSO₂R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂ (C₁₋₄ alkyl), CONH₂, CONH(C₁₋₄ alkyl) and CON(C₁₋₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from:

(c-1-1) halo, hydroxy, OR¹⁴³, S(O)ₘ R¹⁴³, nitro, amino, mono- or di-(C₁₋₄ alkyl)amino, NHSO₂ R¹⁴³, CO₂H, CO₂ (C₁₋₄ alkyl), CONH₂, CONH(C₁₋₄ alkyl), CON(C₁₋₄ alkyl)₂, OC(O)R¹⁴³, thienyl, naphthyl and groups of the following formulae:

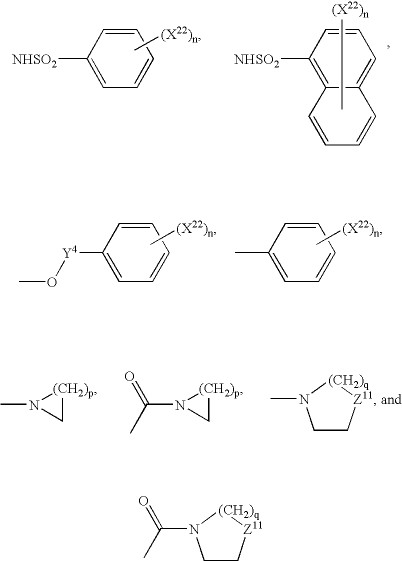

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘ R¹⁴³, amino, mono- or di-(C₁₋₄ alkyl)amino, CONH₂, CONH(C₁₋₄ alkyl) and CON(C₁₋₄ alkyl)₂,

(c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘ R¹⁴³, SO₂ NH₂, SO₂ NH(C₁₋₄ alkyl), SO₂ N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂ (C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘ R¹⁴³, amino, mono- or di-(C₁₋₄ alkyl)amino, CONH₂, CONH(C₁₋₄ alkyl), CON(C₁₋₄ alkyl)₂, CO₂H and CO₂ (C₁₋₄ alkyl), and —Y-phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘ R¹⁴³, amino, mono- or di-(C₁₋₄ alkyl)amino, CO₂H, CO₂ (C₁₋₄ alkyl), CONH₂, CONH(C₁₋₄ alkyl) and CON(C₁₋₄ alkyl)₂,

(c-6) a group of the following formula:

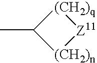

X²² is halo, C₁₋₄ alkyl, hydroxy, C₁₋₄ alkoxy, halosubstitutued C₁₋₄ alkoxy, S(O)ₘ R¹⁴³, amino, mono- or di-(C₁₋₄ alkyl)amino, NHSO₂ R⁴³, nitro, halosubstitutued C₁₋₄ alkyl, CN, CO₂H, CO₂ (C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂; R¹⁴³ is C₁₋₄ alkyl or halosubstituted C₁₋₄ alkyl;

m is 0, 1 or 2; n is 0, 1, 2 or 3; p is 1, 2, 3, 4 or 5; q is 2 or 3;

Z¹¹ is oxygen, sulfur or NR¹⁴⁴; and

R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘ R¹⁴³, amino, mono- or di-(C₁₋₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Materials that can serve as a cyclooxygenase-2 selective inhibitor of the present invention include aryl phenylhydrazides that are described in U.S. Pat. No. 6,077,869. Such aryl phenylhydrazides have the formula shown below in formula XXVIII:

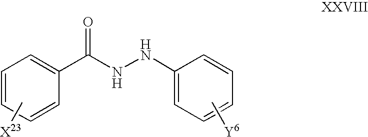

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a cyclooxygenase-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

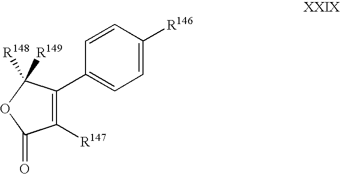

or a pharmaceutical salt thereof,

wherein:

R¹⁴⁶ is selected from the group consisting of SCH₃, —S(O)₂ CH₃ and —S(O)₂ NH₂;

R³ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted With 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a cyclooxygenase-2 selective inhibitor of the present invention include bisaryl compounds that are described in U.S. Pat. No. 5,994,379. Such bisaryl compounds have the formula shown below in formula XXX:

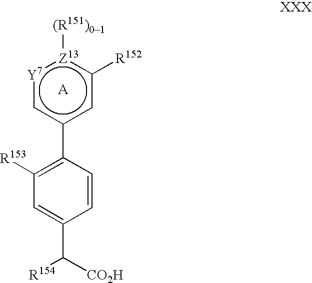

or a pharmaceutically acceptable salt, ester or tautomer thereof,

wherein:

Z¹³ is C or N;

when Z¹³ is N, R¹⁵¹ represents H or is absent, or is taken in conjunction with R as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a cyclooxygenase-2 selective inhibitor of the present invention include 1,5-diarylpyrazoles that are described in U.S. Pat. No. 6,028,202. Such 1,5-diarylpyrazoles have the formula shown below in formula XXXI:

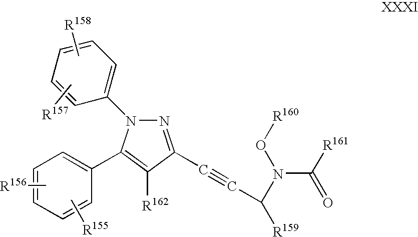

wherein:

R¹⁵⁵, R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or

R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen; and pharmaceutically acceptable salts thereof.

Materials that can serve as a cyclooxygenase-2 selective inhibitor of the present invention include 2-substituted imidazoles that are described in U.S. Pat. No. 6,040,320. Such 2-substituted imidazoles have the formula shown below in formula XXXII:

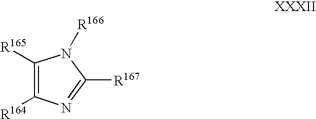

wherein:

R¹⁶⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms, substituted heteroaryl;

wherein the substituents are independently selected from one or more members of the group consisting of C₁₋₅ alkyl and halogen, or substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅ alkyloxycarbonyl, arylC₁₋₅alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or

substituted arylC₁₋₅ alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ—(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₁₅ alkylsulfonyl, phenylsulfonyl,

substituted sulfonamido,

wherein the sulfonyl substituent is selected from the group consisting of C₁₋₅ alkyl, phenyl, araC₁₋₅ alkyl, thienyl, furanyl, and naphthyl; substituted vinyl,

wherein the substituents are independently selected from one or members of the group consisting of fluorine, bromine, chlorine and iodine, substituted ethynyl,

wherein the substituents are independently selected from one or more members of the group consisting of fluorine, bromine chlorine and iodine, substituted C₁₋₅ alkyl,

wherein the substituents are selected from the group consisting of one or more C₁₋₅ alkoxy, trihaloalkyl, phthalimido and amino, substituted phenyl,

wherein the phenyl substituents are independently selected from one or more members of the group consisting of C₁₋₅ alkyl, halogen and C₁₋₅ alkoxy, substituted phenoxy,

wherein the phenyl substituents are independently selected from one or more members of the group consisting of C₁₋₅ alkyl, halogen and C₁₋₅ alkoxy, substituted C₁₋₅ alkoxy,

wherein the alkyl substituent is selected from the group consisting of phthalimido and amino, substituted arylC₁₋₅ alkyl,

wherein the alkyl substituent is hydroxyl, substituted arylC₁₋₅ alkyl,

wherein the phenyl substituents are independently selected from one or more members of the group consisting of C₁₋₅ alkyl, halogen and C₁₋₅ alkoxy, substituted amido,

wherein the carbonyl substituent is selected from the group consisting of C₁₋₅ alkyl, phenyl, arylC₁₋₅ alkyl, thienyl, furanyl, and naphthyl, substituted phenylcarbonyl,

wherein the phenyl substituents are independently selected from one or members of the group consisting of C₁₋₅ alkyl, halogen and C₁₋₅ alkoxy, substituted C₁₋₅ alkylthio,

wherein the alkyl substituent is selected from the group consisting of hydroxy and phthalimido,

substituted C₁₋₅ alkylsulfonyl,

wherein the alkyl substituent is selected from the group consisting of hydroxy and phthalimido, substituted phenylsulfonyl,

wherein the phenyl substituents are independently selected from one or members of the group consisting of bromine, fluorine, chlorine, C₁₋₅ alkoxy and trifluoromethyl, with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X²⁴ cannot be vinyl, ethynyl, C₁₋₅ alkylaminocarbonyl, phenylaminocarbonyl, arylC₁₋₅ alkylaminocarbonyl, C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen; and pharmaceutically acceptable salts thereof.

Materials that can serve as a cyclooxygenase-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

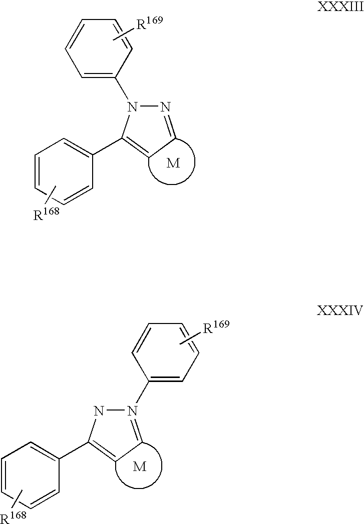

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂ (C₁-C₆)alkyl; and

the fused moiety M is a group selected from the group consisting of an optionally substituted cyclohexyl and cycloheptyl group having the formulae:

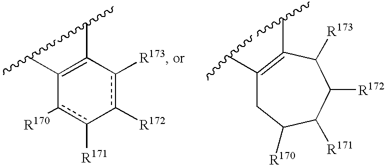

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ON H(CH₃)COCH₂—, —OCOCH.dbd. and —O—;

R¹⁷⁰ and R¹⁷¹ are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂ CH₃, —OSO₂ NHCO₂ CH₃, ═CHCO₂ CH₂ CH₃, —CH₂ CO₂H, —CH₂ CO₂ CH₃, —CH₂ CO₂ CH₂ CH₃, —CH₂ CON(CH₃)₂, —CH₂ CO₂ NHCH₃, —CHCHCO₂ CH₂ CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

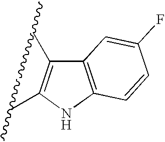

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂ CH₃;

with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a cyclooxygenase-2 selective inhibitor of the present invention include esters derived from indolealkanols and novel amides derived from indolealkylamides that are described in U.S. Pat. No. 6,306,890. Such compounds have the general formula shown below in formula XXXV:

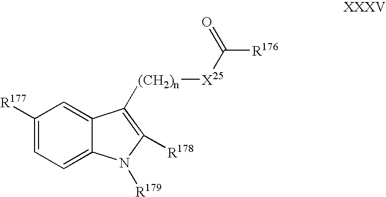

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C₁ to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₈ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo;

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C₁ to C6 alkoxy, C₁ to C₆ branched alkoxy, C4 to C₈ aryloxy, or halo-substituted versions thereof or R¹⁷⁷ is halo where halo is chloro, fluoro, bromo, or iodo;

R¹⁷⁸ is hydrogen, C₁ to C₆ alkyl or C₁ to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C₁ to C₆ branched alkyl.

Materials that can serve as a cyclooxygenase-2 selective inhibitor of the present invention include pyridazinone compounds that are described in U.S. Pat. No. 6,307,047. Such pyridazinone compounds have the formula shown below in formula XXXVI:

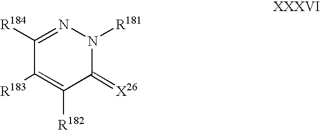

or a pharmaceutically acceptable salt, ester, or prodrug thereof,

wherein:

X²⁶ is selected from the group consisting of O, S, —NR¹⁸⁵, —NOR^{a}, and —NNR^{b}R^{c};

R¹⁸⁵ is selected from the group consisting of alkenyl; alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙ C(O)R¹⁸⁶, —(CH₂)ₙ CH(OH)R¹⁸⁶, —(CH₂)ₙ C(NOR^{d})R¹⁸⁶, —(CH₂)ₙ CH(NOR^{d})R¹⁸⁶, —(CH₂)ₙ CH(NR^{d}R^{e})R¹⁸⁶, —R¹⁸⁷ R¹⁸⁸, —(CH₂)ₙ C≡CR¹⁸⁸, —(CH₂)ₙ [CH(CX^{26′}₃)]ₘ (CH₂)ₚ R¹⁸⁸, —(CH₂)ₙ (CX^{26,}₂)ₘ (CH₂)ₚ R¹⁸⁸, and —(CH₂)ₙ (CHX^{26,}₃)ₘ (CH₂)ₘ R¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl; X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³, or R¹⁸⁴ must be Z¹⁴, and further provided that only one of R¹⁸², R¹⁸³, or R¹⁸⁴ is Z¹⁴;

Z¹⁴ is selected from the group consisting of:

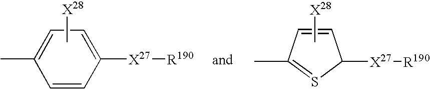

X²⁷ is selected from the group consisting of S(O)₂, S(O)(NR¹⁹¹), S(O), Se(O)₂, P(O)(OR¹⁹²), and P(O)(NR¹⁹³ R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a cyclooxygenase-2 selective inhibitor of the present invention include benzosulphonamide derivatives that are described in U.S. Pat. No. 6,004,948. Such benzosulphonamide derivatives have the formula shown below in formula XXXVII:

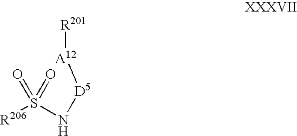

wherein:

A denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

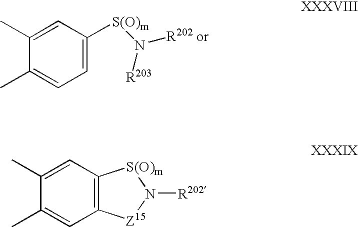

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered,

saturated, partially or totally unsaturated heterocycle with one or more heteroatoms N, O, or S, which may optionally be substituted by oxo, an alkyl, alkylaryl or aryl group or a group (CH₂)ₙ—X²⁹, R²⁰², denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂ R²⁰⁴, —OCO₂ R²⁰⁴, —CN, —CONR²⁰⁴OR²⁰⁵, —CONR²⁰⁴R²⁰⁵, —SR²⁰⁴, —S(O)R²⁰⁴, —S(O)₂ R²⁰⁴, —NR²⁰⁴R²⁰⁵, —NHC(O)R²⁰⁴, —NHS(O)₂R²⁰⁴;

Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂ NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄-alkyl group which may optionally be mono- or polysubstituted by halogen or alkoxy, or R²⁰⁶ denotes CF₃; and

m denotes an integer from 0 to 2;

with the proviso that A1 does not represent O if R²⁰⁶ denotes CF₃;

and the pharmaceutically acceptable salts thereof.

Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

Further preferred COX-2 inhibitors that may be used in the present invention include, but are not limited to:

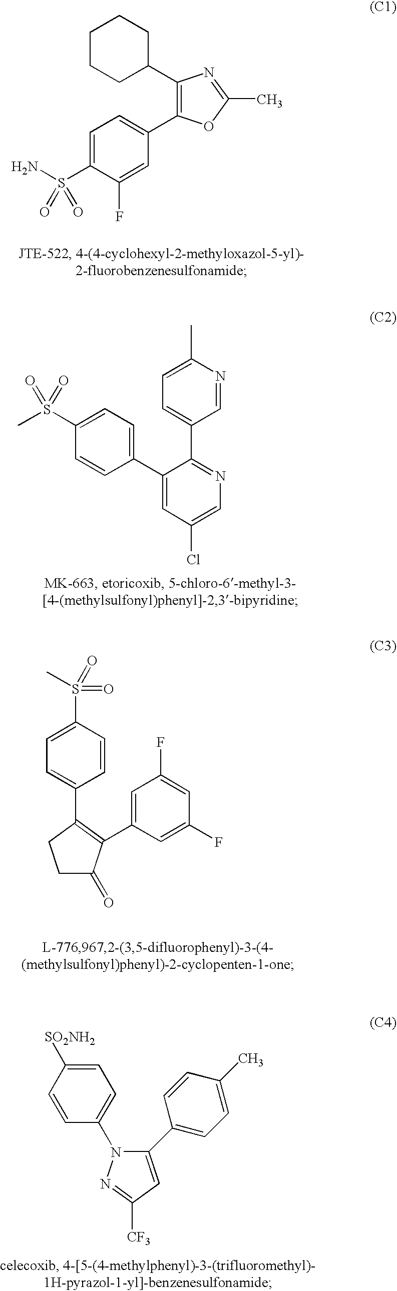

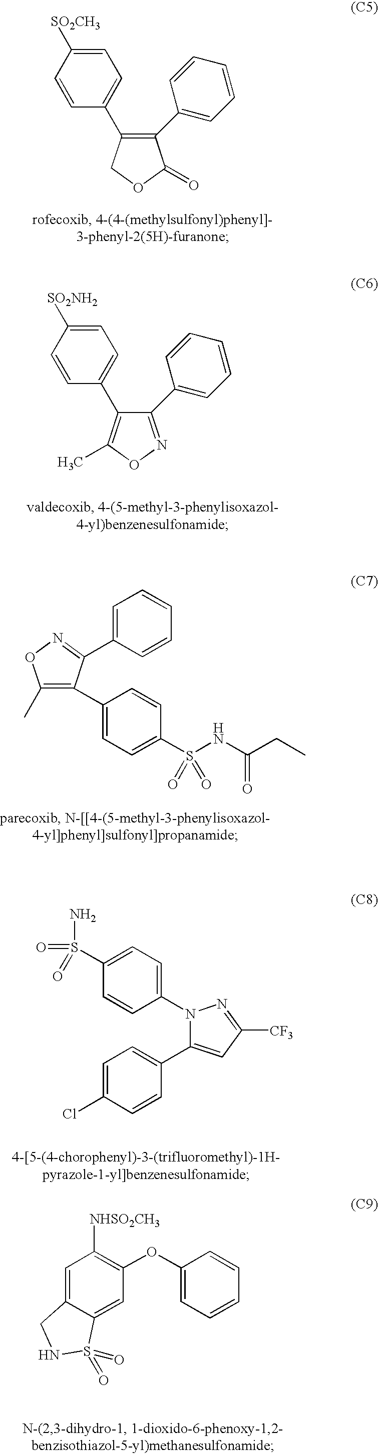

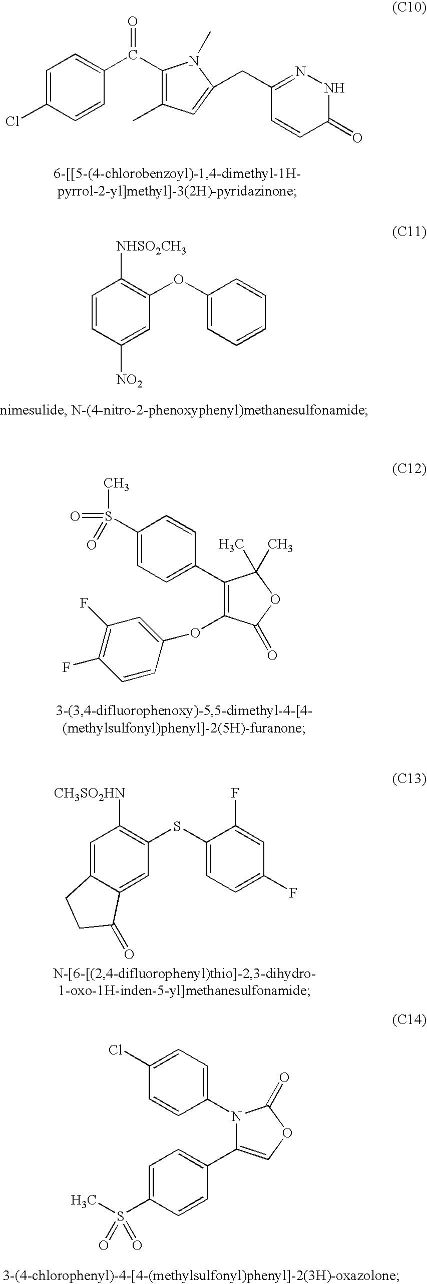

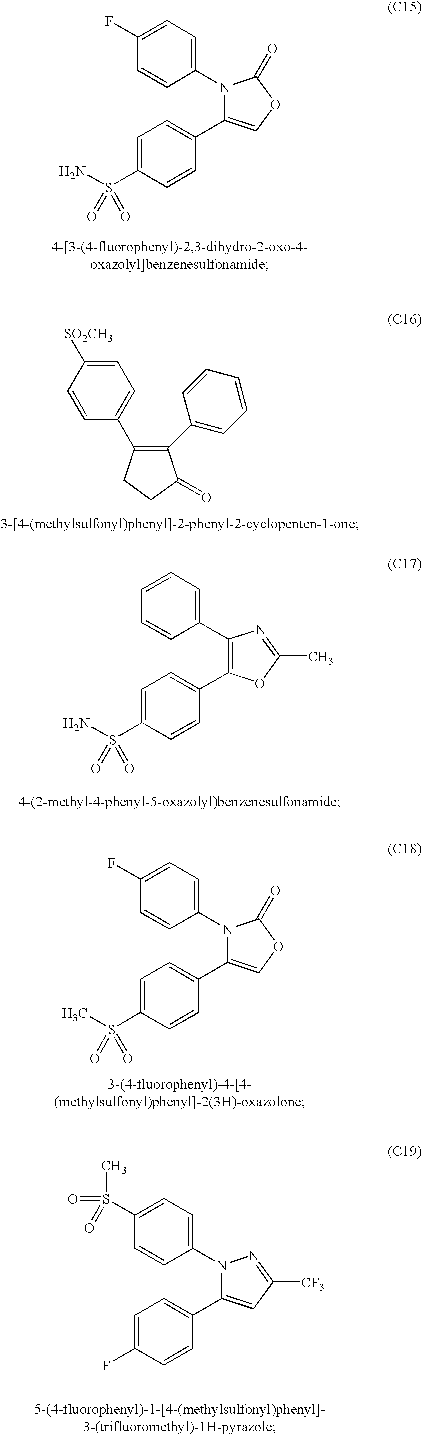

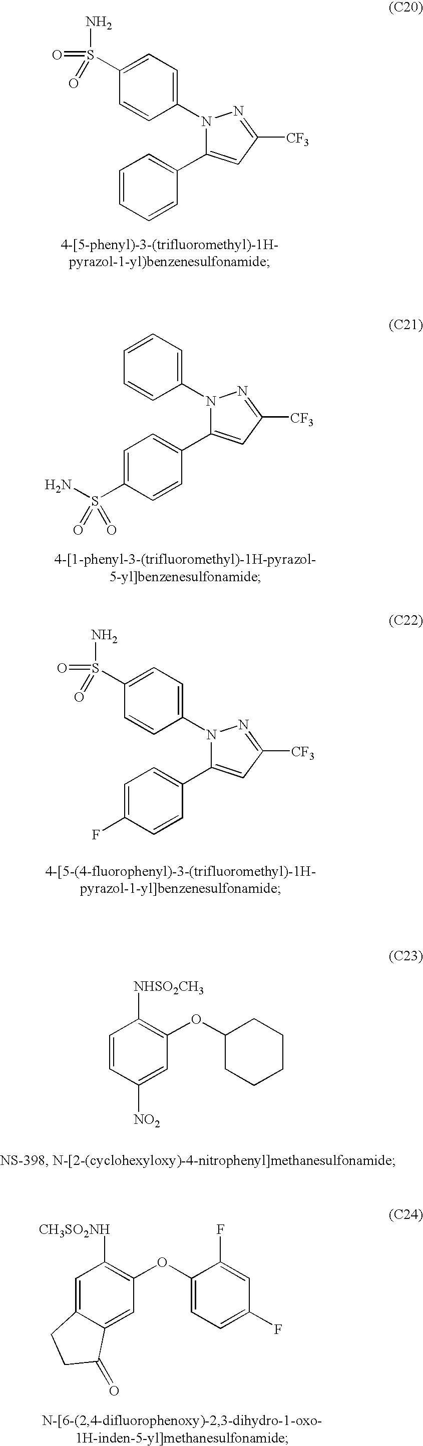

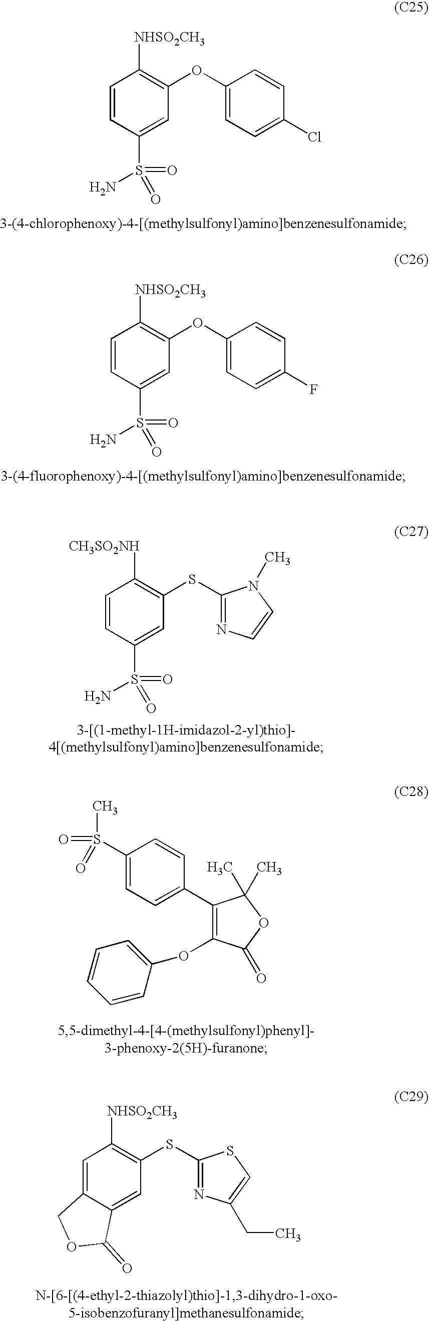

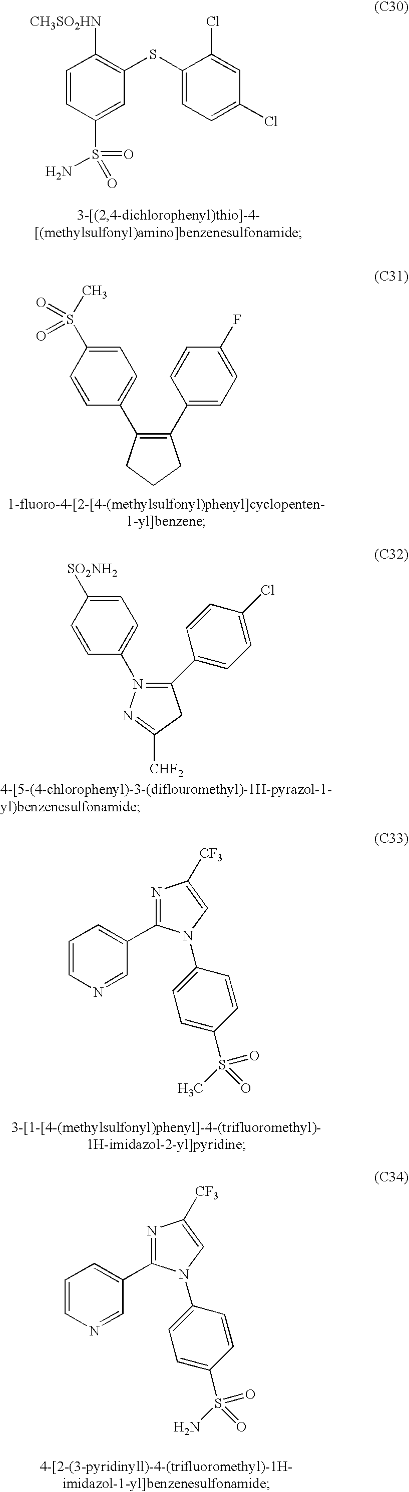

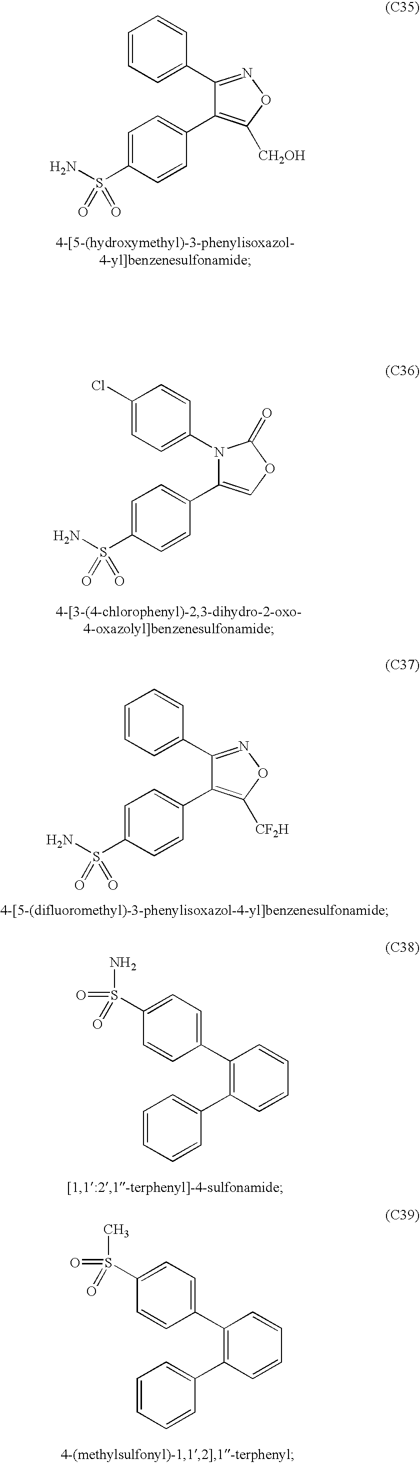

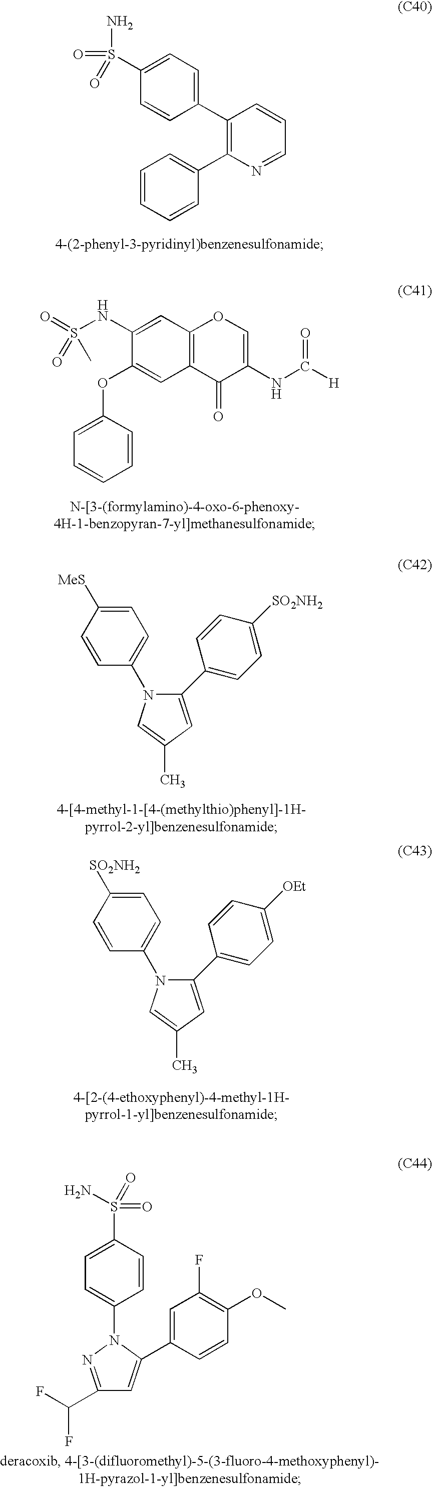

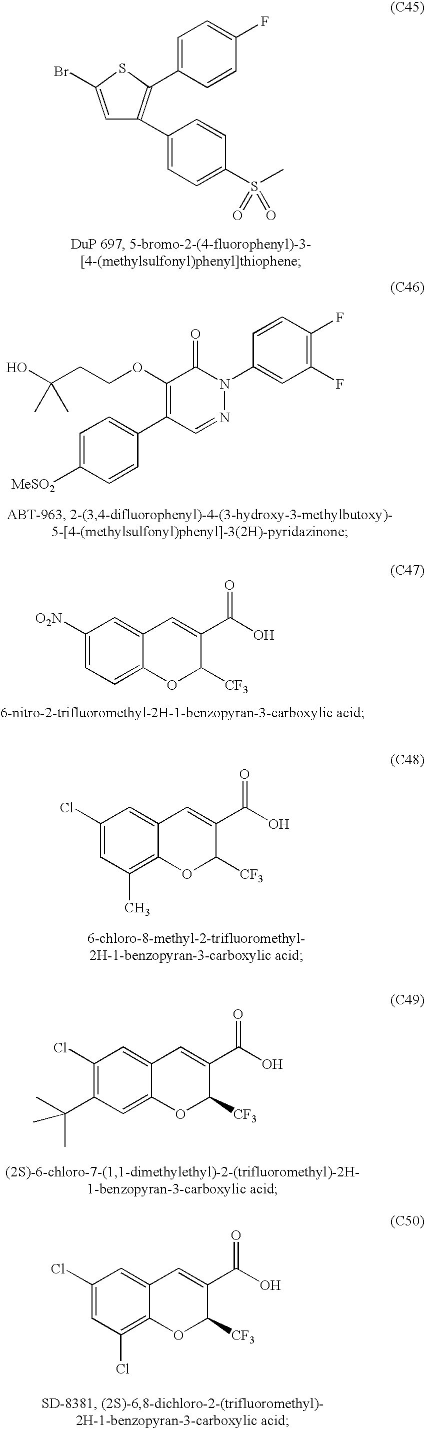

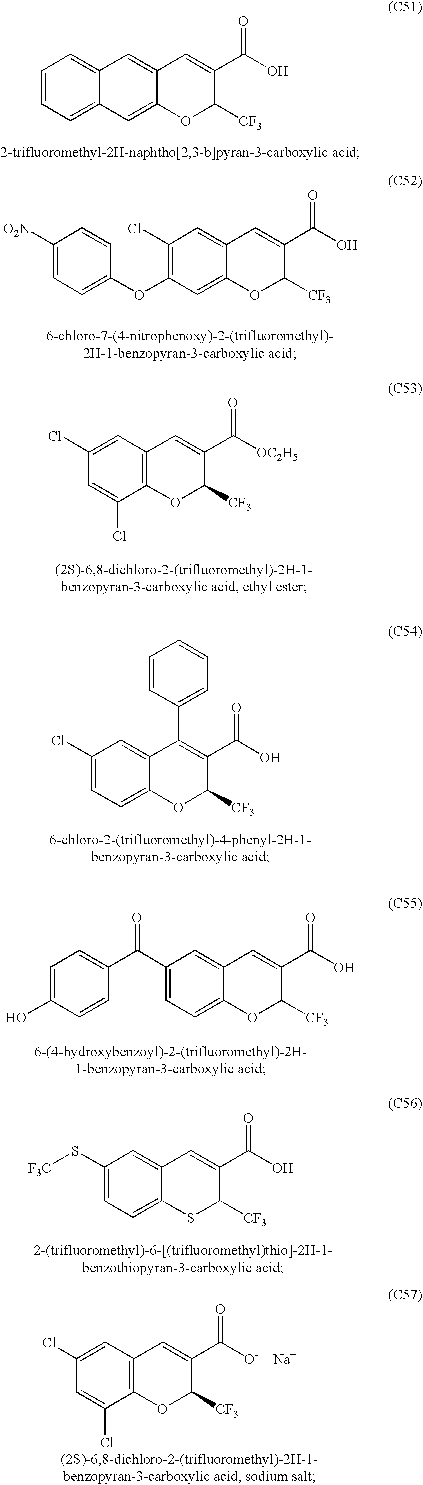

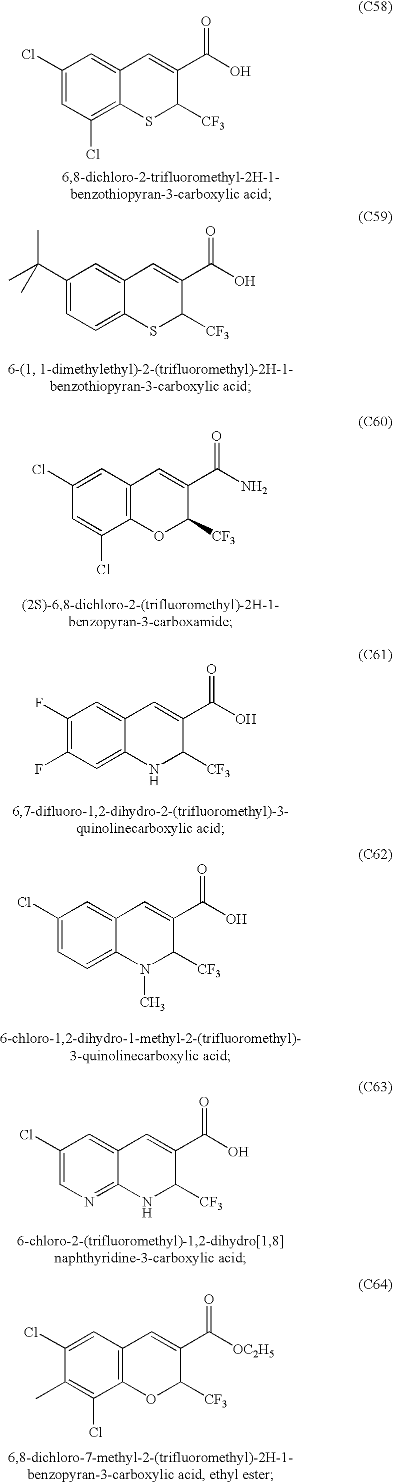

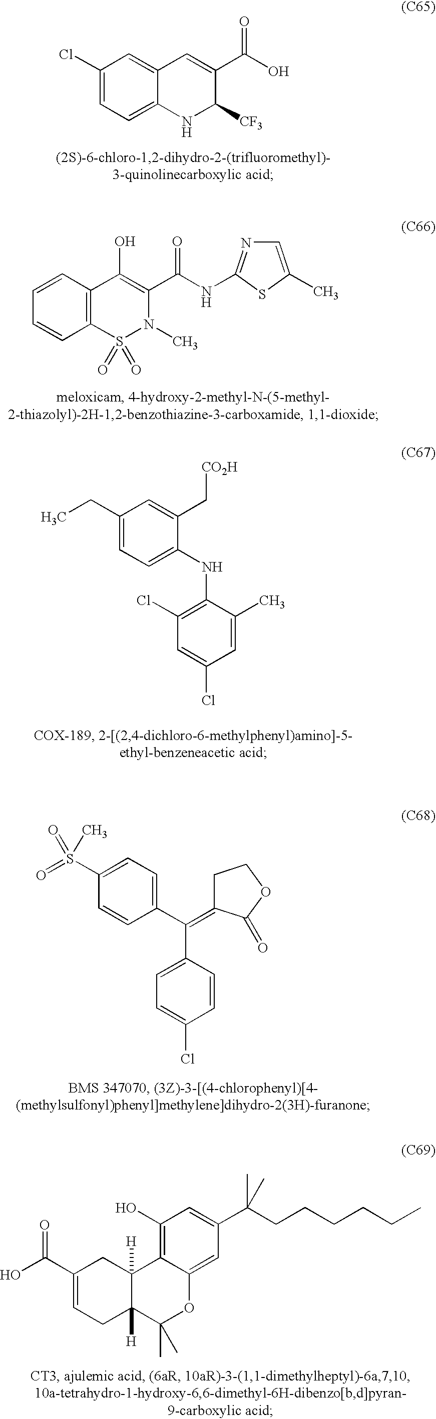

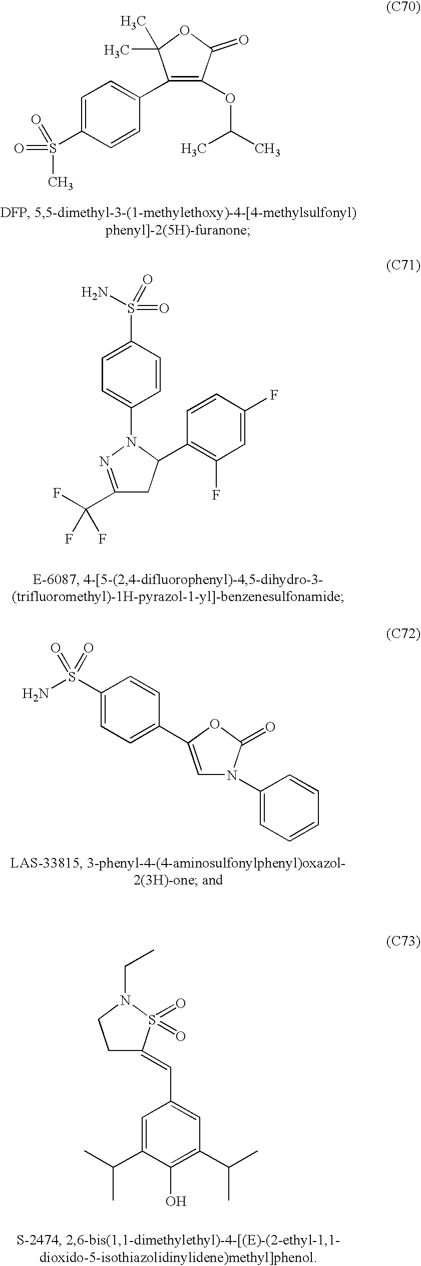

The CAS reference numbers for nonlimiting examples of COX-2 inhibitors are identified in Table No. 3 below.

TABLE No. 3
COX-2 Inhibitor's CAS Reference Numbers

| Compound Number | CAS Reference Number |
|---|---|
| C1 | 180200-68-4 |
| C2 | 202409-33-4 |
| C3 | 212126-32-4 |
| C4 | 169590-42-5 |
| C5 | 162011-90-7 |
| C6 | 181695-72-7 |
| C7 | 198470-84-7 |
| C8 | 170569-86-5 |
| C9 | 187845-71-2 |
| C10 | 179382-91-3 |
| C11 | 51803-78-2 |
| C12 | 189954-13-0 |
| C13 | 158205-05-1 |
| C14 | 197239-99-9 |
| C15 | 197240-09-8 |
| C16 | 226703-01-1 |
| C17 | 93014-16-5 |
| C18 | 197239-97-7 |
| C19 | 162054-19-5 |
| C20 | 170569-87-6 |
| C21 | 279221-13-5 |
| C22 | 170572-13-1 |
| C23 | 123653-11-2 |
| C24 | 80937-31-1 |
| C25 | 279221-14-6 |
| C26 | 279221-15-7 |
| C27 | 187846-16-8 |
| C28 | 189954-16-3 |
| C29 | 181485-41-6 |
| C30 | 187845-80-3 |
| C31 | 158959-32-1 |
| C32 | 170570-29-3 |
| C33 | 177660-77-4 |
| C34 | 177660-95-6 |
| C35 | 181695-81-8 |
| C36 | 197240-14-5 |
| C37 | 181696-33-3 |
| C38 | 178816-94-9 |
| C39 | 178816-61-0 |
| C40 | 279221-17-9 |
| C41 | 123663-49-0 |
| C42 | 197905-01-4 |
| C43 | 197904-84-0 |
| C44 | 169590-41-4 |
| C45 | 88149-94-4 |
| C46 | 266320-83-6 |
| C47 | 215122-43-3 |
| C48 | 215122-44-4 |
| C49 | 215122-74-0 |
| C50 | 215123-80-1 |
| C51 | 215122-70-6 |
| C52 | 264878-87-7 |
| C53 | 279221-12-4 |
| C54 | 215123-48-1 |
| C55 | 215123-03-8 |
| C56 | 215123-60-7 |
| C57 | 279221-18-0 |
| C58 | 215123-61-8 |
| C59 | 215123-52-7 |
| C60 | 279221-19-1 |
| C61 | 215123-64-1 |
| C62 | 215123-70-9 |
| C63 | 215123-79-8 |
| C64 | 215123-91-4 |
| C65 | 215123-77-6 |
| C66 | 71125-38-7 |
| C67 | 220991-33-3 |
| C68 | 197438-41-8 |
| C69 | 137945-48-3 |
| C70 | 189954-66-3 |
| C71 | 251442-94-1 |
| C73 | 158089-95-3 |

Nonlimiting examples of COX-2 inhibitors that may be used in the present invention are identified in Table No. 4 below. The individual references in Table No. 4 are each herein individually incorporated by reference.

TABLE No. 4
COX-2 Inhibitors

| | Trade/ | |
|---|---|---|
| Compound | Research Name | Reference |
| 6-chloro-4-hydroxy-2-methyl-N-2- | lornoxicam; | CAS No. |
| pyridinyl-2H-thieno[2,3-e]-1,2-thiazine-3- | Safem ® | 70374-39-9 |
| carboxamide, 1,1-dioxide | | |
| 1,5-Diphenyl-3-substituted pyrazoles | | WO |
| | | 97/13755 |
| | radicicol | WO |
| | | 96/25928. |
| | | Kwon et al |
| | | (Cancer |
| | | Res(1992) |
| | | 52 6296) |
| | GB-02283745 | |
| | TP-72 | Cancer Res |
| | | 1998 58 4 |
| | | 717-723 |
| 1-(4-chlorobenzoyl)-3-[4-(4-fluoro-phenyl) | A-183827.0 | |
| thiazol-2-ylmethyl]-5-methoxy-2- | | |
| methylindole | | |
| | GR-253035 | |
| 4-(4-cyclohexyl-2-methyloxazol-5-yl)-2- | JTE-522 | JP 9052882 |
| fluorobenzenesulfonamide | | |
| 5-chloro-3-(4-(methylsulfonyl)phenyl)-2- | | |
| (methyl-5-pyridinyl)-pyridine | | |
| 2-(3,5-difluoro-phenyl)-3-4- | | |
| (methylsulfonyl)-phenyl)-2-cyclopenten- | | |
| 1-one | | |
| | L-768277 | |
| | L-783003 | |
| | MK-966; | US |
| | VIOXX ®, | 5968974 |
| | Rofecoxib | |
| indomethacin-derived indolalkanoic acid | | WO |
| | | 96/374679 |
| 1-Methylsulfonyl-4-[1,1-dimethyl-4-(4- | | WO |
| fluorophenyl)cyclopenta-2,4-dien-3- | | 95/30656. |
| yl]benzene | | WO |
| | | 95/30652. |
| | | WO |
| | | 96/38418. |
| | | WO |
| | | 96/38442. |
| 4,4-dimethyl-2-phenyl-3-[4- | | |
| (methylsulfonyl)phenyl]cyclo-butenone | | |
| 2-(4-methoxyphenyl)-4-methyl-1-(4- | | EP 799823 |
| sulfamoylphenyl)-pyrrole | | |
| N-[5-(4-fluoro)phenoxy]thiophene-2- | RWJ-63556 | |
| methanesulfon-amide | | |
| 5(E)-(3,5-di-tert-butyl-4- | | |
| hydroxy)benzylidene-2-ethyl-1,2- | S-2474 | EP 595546 |
| isothiazolidine-1,1-dioxide | | |
| 3-formylamino-7-methylsulfonylamino-6- | T-614 | DE |
| phenoxy-4H-1-benzopyran-4-one | | 3834204 |
| Benzenesulfonamide, 4-(5-(4- | celecoxib | US |
| methylphenyl)-3-(trifluoromethyl)-1H- | | 5466823 |
| pyrazol-1-yl)- | | |
| CS 502 | (Sankyo) | |
| MK 633 | (Merck) | |
| | meloxicam | US |
| | | 4233299 |
| | nimesulide | US |
| | | 3840597 |

The following references listed in Table No. 5 below, hereby individually incorporated by reference, describe various COX-2 inhibitors suitable for use in the present invention described herein, and processes for their manufacture.

TABLE No. 5
COX-2 Inhibitor References

| | | | |
|---|---|---|---|
| WO 99/30721 | WO 99/30729 | US 5760068 | WO 98/15528 |
| WO 99/25695 | WO 99/24404 | WO 99/23087 | FR 27/71005 |
| EP 921119 | FR 27/70131 | WO 99/18960 | WO 99/15505 |
| WO 99/15503 | WO 99/14205 | WO 99/14195 | WO 99/14194 |
| WO 99/13799 | GB 23/30833 | US 5859036 | WO 99/12930 |
| WO 99/11605 | WO 99/10332 | WO 99/10331 | WO 99/09988 |
| US 5869524 | WO 99/05104 | US 5859257 | WO 98/47890 |
| WO 98/47871 | US 5830911 | US 5824699 | WO 98/45294 |
| WO 98/43966 | WO 98/41511 | WO 98/41864 | WO 98/41516 |
| WO 98/37235 | EP 86/3134 | JP 10/175861 | US 5776967 |
| WO 98/29382 | WO 98/25896 | ZA 97/04806 | EP 84/6,689 |
| WO 98/21195 | GB 23/19772 | WO 98/11080 | WO 98/06715 |
| WO 98/06708 | WO 98/07425 | WO 98/04527 | WO 98/03484 |
| FR 27/51966 | WO 97/38986 | WO 97/46524 | WO 97/44027 |
| WO 97/34882 | US 5681842 | WO 97/37984 | US 5686460 |
| WO 97/36863 | WO 97/40012 | WO 97/36497 | WO 97/29776 |
| WO 97/29775 | WO 97/29774 | WO 97/28121 | WO 97/28120 |
| WO 97/27181 | WO 95/11883 | WO 97/14691 | WO 97/13755 |
| WO 97/13755 | CA 21/80624 | WO 97/11701 | WO 96/41645 |
| WO 96/41626 | WO 96/41625 | WO 96/38418 | WO 96/37467 |
| WO 96/37469 | WO 96/36623 | WO 96/36617 | WO 96/31509 |
| WO 96/25405 | WO 96/24584 | WO 96/23786 | WO 96/19469 |
| WO 96/16934 | WO 96/13483 | WO 96/03385 | US 5510368 |
| WO 96/09304 | WO 96/06840 | WO 96/06840 | WO 96/03387 |
| WO 95/21817 | GB 22/83745 | WO 94/27980 | WO 94/26731 |
| WO 94/20480 | WO 94/13635 | FR 27/70,131 | US 5859036 |
| WO 99/01131 | WO 99/01455 | WO 99/01452 | WO 99/01130 |
| WO 98/57966 | WO 98/53814 | WO 98/53818 | WO 98/53817 |
| WO 98/47890 | US 5830911 | US 5776967 | WO 98/22101 |
| DE 19/753463 | WO 98/21195 | WO 98/16227 | US 5733909 |
| WO 98/05639 | WO 97/44028 | WO 97/44027 | WO 97/40012 |
| WO 97/38986 | US 5677318 | WO 97/34882 | WO 97/16435 |
| WO 97/03678 | WO 97/03667 | WO 96/36623 | WO 96/31509 |
| WO 96/25928 | WO 96/06840 | WO 96/21667 | WO 96/19469 |
| US 5510368 | WO 96/09304 | GB 22/83745 | WO 96/03392 |
| WO 94/25431 | WO 94/20480 | WO 94/13635 | JP 09052882 |
| GB 22/94879 | WO 95/15316 | WO 95/15315 | WO 96/03388 |
| WO 96/24585 | US 5344991 | WO 95/00501 | US 5968974 |
| US 5945539 | US 5994381 | US 5521207 | |

Hormonal agents are useful as antineoplastic agents. Aromatase inhibitors, a class of hormonal agents, are useful in the prevention, treatment and inhibition of neoplasia or neoplasia-related orders. Aromatase inhibitors inhibit aromatase (estrogen synthase), a membrane-bound enzyme complex that catalyses the conversion of androgens to estrogens. Since estrogen receptor-positive breast cancers are stimulated to grow by endogenous estrogen, the use of aromatase inhibitors is useful in inhibiting estrogen production, resulting in tumor regression.

Aromatase inhibitor antineoplastic agents are broadly classified as steroidal and nonsteroidal. The majority of aromatase inhibitors known are steroidal compounds that are structurally related to the natural substrate of aromatase. Examples of steroidal aromatase inhibitors include formestane, exemestane, and atamestane. Nonsteroidal inhibitors have a heteroatom, usually in a nitrogen-containing heterocyclo, as a common feature that interferes with the steroidal hydroxylation of the aromatase enzyme. Examples of nonsteroidal aromatase inhibitors include rogletimide, letrozole and anastrozole.

Suitable aromatase inhibitors that may be used in the present invention include, but are not limited to aminoglutethimide; anastrozole; exemestane; fadrozole; formestane; letrozole; liarozole; vorozole; and Yamanouchi YM-511.

Some aromatase inhibitors that may be used in the methods, combinations and compositions of the present invention include, but are not limited to, those identified in Table No. 6 below.

TABLE No. 6
Aromatase Inhibitors

| | Common | | | |
|---|---|---|---|---|
| | Name/Trade | | | |
| Compound | Name | Company | Reference | Dosage |
| | letrozole | | US 4749346 | |
| Androst-4-ene-3,6,17- | NKS01; | Snow Brand | EP 300062 | |
| trione, 14-hydroxy- | 14alpha- | | | |
| | OHAT; | | | |
| | 14OHAT | | | |
| 4-[N-(4-bromobenzyl)-N- | YM-511 | Yamanou-chi | | |
| (4-cyanophenyl)amino]- | | | | |
| 4H-1,2,4-triazole | | | | |
| 2,6-Piperidinedione, 3-(4- | aminoglutethimide; | Novartis | US 3944671 | |
| aminophenyl)-3-ethyl- | Ciba- | | | |
| | 16038; | | | |
| | Cytadren; | | | |
| | Elimina; | | | |
| | Orimeten; | | | |
| | Orimet-ene; | | | |
| | Orimetine | | | |
| 1,3- | anastro-zole; | Zeneca | EP 296749 | 1 mg/day |
| Benzenediacetonitrile, alpha, | Arimidex; ICI- | | | |
| alpha, alpha′, alpha′- | D1033; ZD- | | | |
| tetramethyl-5-(1H-1,2,4- | 1033 | | | |
| triazol-1-ylmethyl)- | | | | |
| Androsta-1,4-diene-3,17- | exemes-tane; | Pharmacia & | DE 3622841 | 5 mg/kg |
| dione, 6-methylene- | FCE-24304 | Upjohn | | |
| Benzonitrile, 4-(5,6,7,8- | fadrozo-le; | Novartis | EP 165904 | 1 mg po bid |
| tetrahydroimidazo[1,5- | Afema; | | | |
| a]pyridin-5-yl)-, | Aresin; CGS- | | | |
| monohydrochloride | 16949; CGS- | | | |
| | 16949A; | | | |
| | CGS-20287; | | | |
| | fadrozole | | | |
| | monohydrochloride | | | |
| Androst-4-ene-3,17- | formest-ane; | Novartis | EP 346953 | 250 or |
| dione, 4-hydroxy- | 4-HAD; 4- | | | 600 mg/wk po |
| | OHA; CGP- | | | |
| | 32349; CRC- | | | |
| | 82/01; Depot; | | | |
| | Lentaron | | | |
| Benzonitrile, 4,4′-(1H- | letroz-ole; | Novartis | EP 236940 | 2.5 mg/day |
| 1,2,4-triazol-1- | CGS-20267; | | | |
| ylmethylene)bis- | Femara | | | |
| 1H-Benzimidazole, 5-[(3- | liaro-zole; | Johnson & | EP 260744 | 300 mg bid |
| chlorophenyl)-1H- | Liazal; Liazol; | Johnson | | |
| imidazol-1-ylmethyl]- | liaro-zole | | | |
| | fumarate; R- | | | |
| | 75251; R- | | | |
| | 85246; Ro- | | | |
| | 85264 | | | |
| 1H-Benzotriazole, 6-[(4- | vorozole; R- | Johnson & | EP 293978 | 2.5 mg/day |
| chlorophenyl)-1H-1,2,4- | 76713; R- | Johnson | | |
| triazol-1-ylmethyl]-1- | 83842; Rivizor | | | |
| methyl- | | | | |

The structures of preferred aromatase inhibitors are listed in Table No. 7 below.

TABLE No. 7
Aromatase Inhibitor Structures

| Compound | |
|---|---|
| Number | Structure |
| A1 | 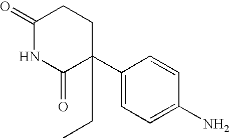 |
| A2 | 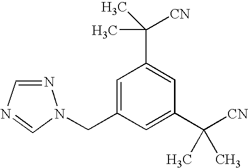 |
| A3 | 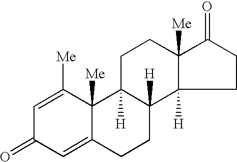 |
| A4 | 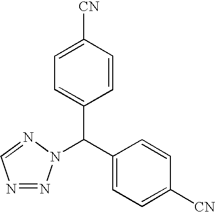 |
| A5 | 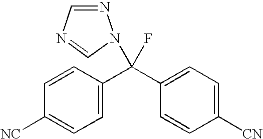 |
| A6 | 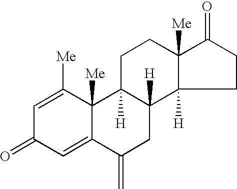 |
| A7 | 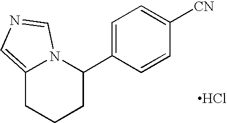 |
| A8 | 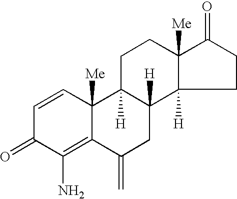 |
| A9 | 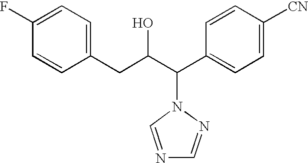 |
| A10 | 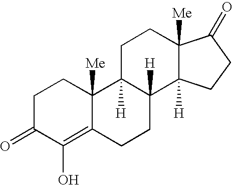 |
| A11 | 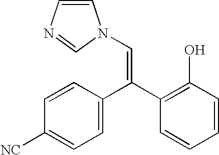 |
| A12 | 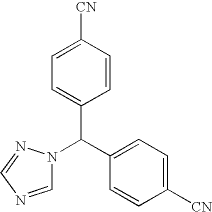 |
| A13 | 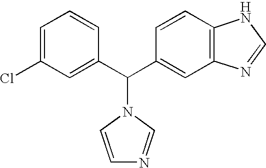 |
| A14 | 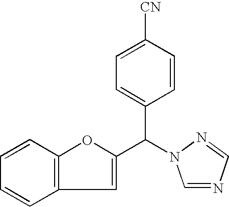 |
| A15 | 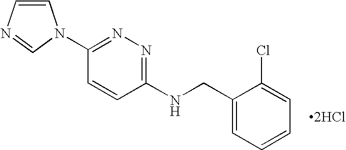 |
| A16 | 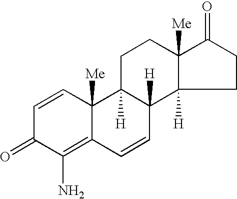 |
| A17 | 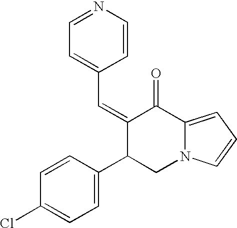 |
| A18 | 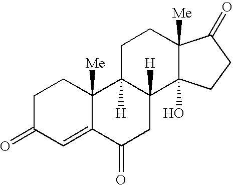 |
| A19 | 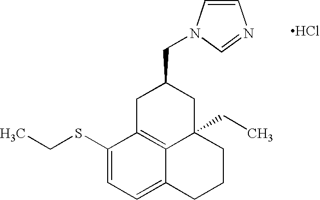 |
| A20 | 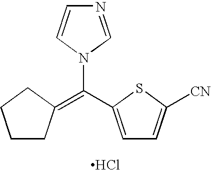 |
| A21 | 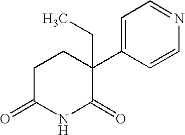 |
| A22 | 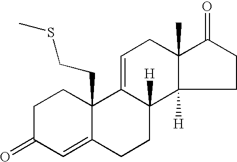 |
| A23 | 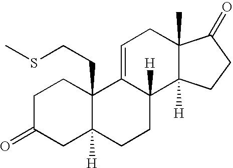 |
| A24 | 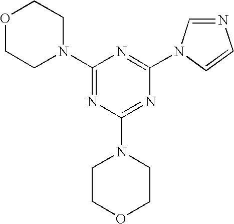 |
| A25 | 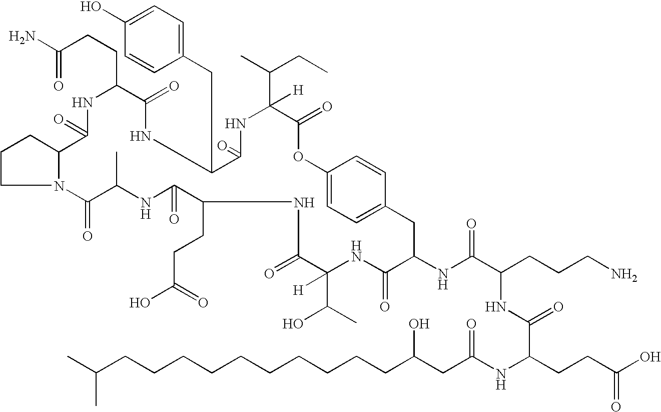 |
| A26 | 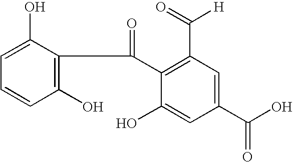 |
| A27 | 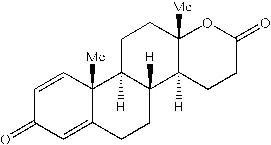 |
| A28 | 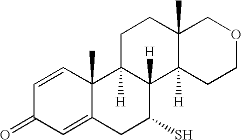 |
| A29 | 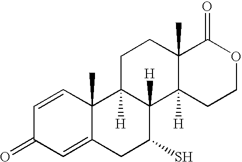 |
| A30 | 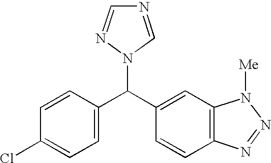 |
| A31 | 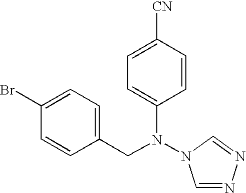 |
| A32 | 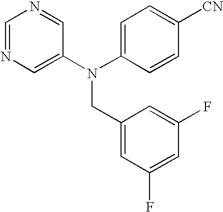 |

The names, CAS registry numbers and references for preferred aromatase inhibitors are listed in Table No. 8 below. The individual references in Table No. 8 are each herein individually incorporated by reference.

TABLE No. 8
Aromatase Inhibitor Antineoplastic Agent Names, CAS Registry
Numbers and References

| Compound | | | |
|---|---|---|---|
| Number | Name(s) | CAS Registry Number | Reference |
| A1 | Aminoglutethimide | 125-84-8 | US 2848455 |
| A2 | Anastrozole | 120511-73-1 | US 4935437 |
| A3 | Atamestane | 96301-34-7 | US 4591585 |
| A4 | CGP-45688, 4,4′-(2H- | 134520-88-0 | EP 408509 |
| | tetrazol-2-ylmethylene)bis- | | |
| | benzonitrile | | |
| A5 | CGS-47645, 4,4′-(fluoro-1H- | 143030-47-1 | US 5227393 |
| | 1,2,4-triazol-1- | | |
| | ylmethylene)bis-benzonitrile | | |
| A6 | Exemestane | 107868-30-4 | US 4808616 |
| A7 | Fadrozole | 102676-47-1 | US 4588732 |
| A8 | FCE-27993, 4-amino-6- | 115837-67-7 | US 5457097 |
| | methylene-androsta-1,4- | | |
| | diene-3,17-dione | | |
| A9 | Finrozole | 204714-56-7 | WO 9413645 |
| A10 | Formestane | 566-48-3 | US 4235893 |
| A11 | 4-[1-(2-Hydroxyphenyl)-2- | 194939-73-6 | JP 09202776 |
| | (1H-imidazol-1- | | |
| | yl)ethenyl]benzo-nitrile | | |
| A12 | Letrozole | 112809-51-5 | US 4749713 |
| A13 | Liarozole | 145858-52-2 | US 4859684 |
| A14 | MEN-11066, 4-(2- | 207288-29-7 | WO 9818791 |
| | benzofuranyl-1H-1,2,4- | | |
| | triazol-1- | | |
| | ylmethyl)benzonitrile | | |
| A15 | MFT-279, N-[(2- | 124079-28-3 | JP 01139578 |
| | chlorophenyl)methyl]-6-(1H- | | |
| | imidazol-1-yl)-3- | | |
| | pyridazinamine, | | |
| | dihydrochloride | | |
| A16 | Minamestane | 105051-87-4 | US 4757061 |
| A17 | MR-20492, (7Z)-6-(4- | 209529-76-0 | P. Auvray, et |
| | chlorophenyl)-6,7-dihydro-7- | | al., J. Steroid |
| | (4-pyridinylmethylene)- | | Biochem. Mol. |
| | 8(5H)-indolizinone | | Biol. (1999), |
| | | | 70(1-3), 59-71 |
| A18 | NKS-01, 14-hydroxy- | 120051-39-0 | US 5098535 |
| | androst-4-ene-3,6,17-trione | | |
| A19 | Org-33201, 1-[[(2S,3aR)-3a- | 148714-92-5 | J. A. A. |
| | ethyl-9-(ethylthio)- | | Geelen, et al., |
| | 2,3,3a,4,5,6-hexahydro-1H- | | J. Steroid |
| | phenalen-2-yl]methyl]-1H- | | Biochem. Mol. |
| | imidazole, | | Biol. (1993), |
| | monohydrochloride | | 44(4-6), 681-2. |
| A20 | Pentrozole | 212894-59-2 | WO 9101975 |
| A21 | Rogletimide | 92788-10-8 | US 5071857 |
| A22 | RU-54115, 10-[2- | 137437-16-2 | EP 434570 |
| | (methylthio)ethyl]-estra- | | |
| | 4,9(11)-diene-3,17-dione | | |
| A23 | RU-56152, 10-[2- | 137437-60-6 | US 5086047 |
| | (methylthio)ethyl]-estr-9(11)- | | |
| | ene-3,17-dione | | |
| A24 | SEF-19, 2-(1H-imidazol-1- | 153429-67-5 | WO 9317009 |
| | yl)-4,6-di-4-morpholinyl- | | |
| | 1,3,5-triazine | | |
| A25 | SNA-60-367, N-(3-hydroxy- | 193738-68-0 | Ken-lchi |
| | 14-methyl-1-oxopentadecyl)- | | Kimura, et al., |
| | □-glutamylornithyl- | | J. Antibiot. |
| | tyrosylthreonyl-□- | | (1997), 50(6), |
| | glutamylalanylprolyl- | | 529-531 |
| | glutaminyltyrosyl-, (10□3)- | | |
| | lactone | | |
| A26 | TAN-931, 4-(2,6- | 127448-92-4 | US 5013757 |
| | dihydroxybenzoyl)-3-formyl- | | |
| | 5-hydroxy-benzoic acid | | |
| A27 | Testolactone | 968-93-4 | US 2744120 |
| A28 | TZA-2209, (4aS,4bR,5R,- | 159821-93-9 | US 5539127 |
| | 10aR,10bS,12aS)- | | |
| | 1,3,4,4a,4b,5,6,10a,- | | |
| | 10b,11,12,12a-dodecahydro- | | |
| | 5-mercapto-10a,12a- | | |
| | dimethyl-8H-phenanthro[2,1- | | |
| | c]pyran-8-one | | |
| A29 | TZA-2237, (4aS,4bR,5R,- | 159822-03-4 | US 5539127 |
| | 10aR,10bS,12aS)- | | |
| | 3,4,4a,5,6,10a,- | | |
| | 10b,11,12,12a-decahydro-5- | | |
| | mercapto-10a,12a-dimethyl- | | |
| | 1H-phenanthro[2,1-c]pyran- | | |
| | 1,8(4bH)-dione | | |
| A30 | Vorozole | 118949-22-7 | US 4943574 |
| A31 | YM-511, 4-[[(4- | 148869-05-0 | US 5674886 |
| | bromophenyl)methyl]-4H- | | |
| | 1,2,4-triazol-4-ylamino]- | | |
| | benzonitrile | | |
| A32 | YM-553, 4-[[(3,5- | 157911-98-3 | US 5538976 |
| | difluorophenyl)methyl]-5- | | |
| | pyrimidinylamino]- | | |
| | benzonitrile | | |

The anastrozole used in the therapeutic combinations of the present invention can be prepared in the manner set forth in U.S. Pat. No. 4,935,437. The letrozole used in the therapeutic combinations of the present invention can be prepared in the manner set forth in U.S. Pat. No. 4,749,713.

More preferred aromatase inhibitors are selected from the group consisting of aminoglutethimide, anastrozole, atamestane, exemestane, fadrozole, finrozole, formestane, letrozole, testolactone, and 4-[[(4-bromophenyl)methyl]-4H-1,2,4-triazol-4-ylamino]benzonitrile.

The compounds useful in the present invention can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

Also included in the methods, combinations and compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Also included in the methods, combinations and compositions of the present invention are the prodrugs of the described compounds and the pharmaceutically-acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe. A nonlimiting example of a “prodrug” that can be used in the methods, combinations and compositions of the present invention is parecoxib, (N-[[4-(5-methyl-3-phenyl-4-isoxazolyl)phenyl]sulfonyl]propanamide).

The methods and combinations of the present invention are useful for the treatment, prevention or inhibition of neoplasia or a neoplasia-related disorder including malignant tumor growth, benign tumor growth and metastasis.

Malignant tumor growth locations comprise the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region.

Malignant tumor growth locations in the nervous system comprise the brain and spine.

Malignant tumor growth locations in the respiratory tract system comprise the lung and bronchus.

Malignant tumor growths in the lymphatic system comprise Hodgkin's lymphoma and non-Hodgkin's lymphoma.

Malignant tumor growth locations in the hepatic system comprise the liver and intrahepatic bile duct.

Malignant tumor growth locations in the musculoskeletal system comprise bone, bone marrow, joint, muscle and connective tissue.

Malignant tumor growth locations in the digestive tract comprise the colon, small intestine, large intestine, stomach, colorectal, pancreas, liver, and rectum.

Malignant tumor growth locations in the renal system comprise the kidney and renal pelvis.

Malignant tumor growth locations in the male reproductive system comprise the prostate, penis and testicle.

Malignant tumor growth locations in the female reproductive system comprise the ovary and cervix.

Malignant tumor growth locations in the urinary tract comprise the bladder, urethra, and ureter.

Malignant tumor growth locations in the nasal sytem comprise the nasal tract and sinuses.

Malignant tumor growth locations in the gastrointestinal tract comprise the esophagus, gastric fundus, gastric antrum, duodenum, hepatobiliary, ileum, jejunum, colon, and rectum.

Malignant tumor growth in the dermis comprises melanoma and basal cell carcinoma.

Malignant tumor growth locations in the head and neck region comprise the mouth, pharynx, larynx, thyroid, and pituitary.

Malignant tumor growth locations further comprise smooth muscle, striated muscle, and connective tissue.

Malignant tumor growth locations even further comprise endothelial cells and epithelial cells.

Malignant tumor growth may be breast cancer.

Malignant tumor growth may be in soft tissue.

Malignant tumor growth may be a viral-related cancer, including cervical, T cell leukemia, lymphoma, and Kaposi's sarcoma.

Benign tumor growth locations comprise the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region.

Benign tumor growth locations in the nervous system comprise the brain and spine.

Benign tumor growth locations in the respiratory tract system comprise the lung and bronchus.

A benign tumor growth in the lymphatic system may comprise a cyst.

Benign tumor growth locations in the hepatic system comprise the liver and intrahepatic bile duct.

Benign tumor growth locations in the musculoskeletal system comprise bone, bone marrow, joint, muscle and connective tissue.

Benign tumor growth locations in the digestive tract comprise the colon, small intestine, large intestine, stomach, colorectal, pancreas, liver, and rectum.

A benign tumor growth in the digestive tract may comprise a polyp.

Benign tumor growth locations in the renal system comprise the kidney and renal pelvis.

Benign tumor growth locations in the male reproductive system comprise the prostate, penis and testicle.

Benign tumor growth in the female reproductive system may comprise the ovary and cervix.

Benign tumor growth in the female reproductive system may comprise a fibroid tumor, endometriosis or a cyst.

Benign tumor growth in the male reproductive system may comprise benign prostatic hypertrophy (BPH) or prostatic intraepithelial neoplasia (PIN).

Benign tumor growth locations in the urinary tract comprise the bladder, urethra, and ureter.

Benign tumor growth locations in the nasal sytem comprise the nasal tract and sinuses.

Benign tumor growth locations in the gastrointestinal tract comprise the esophagus, gastric fundus, gastric antrum, duodenum, hepatobiliary, ileum, jejunum, colon, and rectum.

Benign tumor growth locations in the head and neck region comprise the mouth, pharynx, larynx, thyroid, and pituitary.

Benign tumor growth locations further comprise smooth muscle, striated muscle, and connective tissue.

Benign tumor growth locations even further comprise endothelial cells and epithelial cells.

Benign tumor growth may be located in the breast and may be a cyst or fibrocystic disease.

Benign tumor growth may be in soft tissue.

Metastasis may be from a known primary tumor site or from an unknown primary tumor site.

Metastasis may be from locations comprising the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region.

Metastasis from the nervous system may be from the brain, spine, or spinal cord.

Metastasis from the circulatory system may be from the blood or heart.

Metastasis from the respiratory system may be from the lung or broncus.

Metastasis from the lymphatic system may be from a lymph node, lymphoma, Hodgkin's lymphoma or non-Hodgkin's lymphoma.

Metastasis from the heptatic system may be from the liver or intrahepatic bile duct.

Metastasis from the musculoskeletal system may be from locations comprising the bone, bone marrow, joint, muscle, and connective tissue.

Metastasis from the digestive tract may be from locations comprising the colon, small intestine, large intestine, stomach, colorectal, pancreas, gallbladder, liver, and rectum.

Metastasis from the renal system may be from the kidney or renal pelvis.

Metastasis from the male reproductive system may be from the prostate, penis or testicle.

Metastasis from the female reproductive system may be from the ovary or cervix.

Metastasis from the urinary tract may be from the bladder, urethra, or ureter.

Metastasis from the gastrointestinal tract may be from locations comprising the esophagus, esophagus (Barrett's), gastric fundus, gastric antrum, duodenum, hepatobiliary, ileum, jejunum, colon, and rectum.

Metastasis from the dermis may be from a melanoma or a basal cell carcinoma.

Metastasis from the head and neck region may be from locations comprising the mouth, pharynx, larynx, thyroid, and pituitary.

Metastasis may be from locations comprising smooth muscle, striated muscle, and connective tissue.

Metastasis may be from endothelial cells or epithelial cells.

Metastasis may be from breast cancer.

Metastasis may be from soft tissue.

Metastasis may be from a viral-related cancer, including cervical, T cell leukemia, lymphoma, or Kaposi's sarcoma.

Metastasis may be from tumors comprising a carcinoid tumor, gastrinoma, sarcoma, adenoma, lipoma, myoma, blastoma, carcinoma, fibroma, or adenosarcoma.

Malignant or benign tumor growth may be in locations comprising the genital system, digestive system, breast, respiratory system, urinary system, lymphatic system, skin, circulatory system, oral cavity and pharynx, endocrine system, brain and nervous system, bones and joints, soft tissue, and eye and orbit.

Metastasis may be from locations comprising the genital system, digestive system, breast, respiratory system, urinary system, lymphatic system, skin, circulatory system, oral cavity and pharynx, endocrine system, brain and nervous system, bones and joints, soft tissue, and eye and orbit.

The methods and compositions of the present invention may be used for the treatment, prevention or inhibition of neoplasia or neoplasia-related disorders including acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, benign cysts, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, cysts of the female reproductive system, digestive system cancer, digestive tract polyps, duodenum cancer, endocrine system cancer, endodermal sinus tumor, endometrial hyperplasia, endometrial stromal sarcoma, endometrioid adenocarcinoma, endometriosos, endothelial cell cancer, ependymal cancer, epithelial cell cancer, esophagus cancer, Ewing's sarcoma, eye and orbit cancer, female genital cancer, fibroid tumors, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous carcinoma, squamous cell carcinoma, stomach cancer, striated muscle cancer, submesothelial cancer, superficial spreading melanoma, T cell leukemia, testis cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, vipoma, vulva cancer, well differentiated carcinoma, and Wilm's tumor.

The methods, combinations and compositions of the present invention will be useful for the treatment or prevention of a neoplasia disorder where the neoplasia disorder is located in a tissue of the mammal. The tissues where the neoplasia disorder may be located comprise the lung, breast, skin, stomach, intestine, esophagus, bladder, head, neck, brain, cervical, prostate or ovary of the mammal.

The methods and combinations of the present invention are preferred for the treatment, prevention or inhibition of prostate cancer.

The methods and combinations of the present invention are useful for the treatment, prevention or inhibition of osteoporosis. Osteoporosis may be treated, prevented or inhibited by enhancing the formation of new bone or by reducing or preventing the reabsorption of old bone by the body. Osteoporosis may be evaluated by bone mineral density testing performed by dual-energy X-ray absorptiometry to give a quantitative measure for the demineralization of the bones. A spine CT can show demineralization and quantitative computerized tomography (QCT) can evaluate bond density. Measurement of urinary N-telopeptide (Osteomark) can evaluate bone turnover.

The benefits of treating, preventing or inhibiting osteoporosis include the prevention of brittle, fragile bones that are subject to fracture, particularly of the vertebrae, wrists or hips. Hip fractures are particularly debilitating, leaving about 50% of victims unable to independently walk and is one of the major reasons for admittance to nursing homes. Other symptoms of osteoporosis that may be prevented or alleviated by the compositions and methods of the present invention are low back pain, neck pain, bone pain or tenderness, loss of height over time and stooped posture.

The phrase “neoplasia disorder effective” is intended to qualify the amount of each agent that will achieve the goal of improvement in neoplastic disease severity and the frequency of a neoplastic disease event over treatment of each agent by itself, while avoiding adverse side effects typically associated with alternative therapies.

The phrase “therapeutically effective” is intended to qualify the amount of each agent that will achieve the goal of improvement in neoplastic or osteoporotic disease severity and the frequency of a neoplastic or osteoporotic disease event over treatment of each agent by itself, while avoiding adverse side effects typically associated with alternative therapies.

A “neoplasia disorder effect” or “neoplasia disorder effective amount” is intended to qualify the amount of a COX-2 inhibiting agent and an aromatase inhibitor required to treat, prevent or inhibit a neoplasia disorder or relieve to some extent or one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; or 7) relieving or reducing the side effects associated with the administration of anticancer agents.

A “therapeutically effective amount” is intended to qualify the amount of a COX-2 inhibiting agent and an aromatase inhibitor required to treat, prevent or inhibit osteoporosis, a neoplasia or a neoplasia-related disorder.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “prevention,” in relation to neoplasia, tumor growth or tumor cell growth, means no tumor or tumor cell growth if none had occurred, no further tumor or tumor cell growth if there had already been growth.

The term “chemoprevention” refers to the use of agents to arrest or reverse the chronic cancer disease process in its earliest stages before it reaches its terminal invasive and metastatic phase.

The term “clinical tumor” includes neoplasms that are identifiable through clinical screening or diagnostic procedures including, but not limited to, palpation, biopsy, cell proliferation index, endoscopy, mammagraphy, digital mammography, ultrasonography, computed tomagraphy (CT), magnetic resonance imaging (MRI), positron emmission tomography (PET), radiography, radionuclide evaluation, CT- or MRI-guided aspiration cytology, and imaging-guided needle biopsy, among others. Such diagnostic techniques are well known to those skilled in the art and are described in Cancer Medicine 4th Edition, Volume One. J. F. Holland, R. C. Bast, D. L. Morton, E. Frei III, D. W. Kufe, and R. R. Weichselbaum (Editors). Williams & Wilkins, Baltimore (1997).

The phrases “low dose” or “low dose amount”, in characterizing a therapeutically effective amount of the COX-2 inhibitor and the aromatase inhibitor in the combination therapy, defines a quantity of such agent, or a range of quantity of such agent, that is capable of improving osteoporotic or neoplastic disease severity while reducing or avoiding one or more antineoplastic-agent-induced side effects, such as myelosupression, cardiac toxicity, alopecia, nausea or vomiting.

The phrase “adjunctive therapy” encompasses treatment of a subject with agents that reduce or avoid side effects associated with the combination therapy of the present invention, including, but not limited to, those agents, for example, that reduce the toxic effect of anticancer drugs, e.g., bone resorption inhibitors, cardioprotective agents; prevent or reduce the incidence of nausea and vomiting associated with chemotherapy, radiotherapy or operation; or reduce the incidence of infection associated with the administration of myelosuppressive anticancer drugs.

The phrase a “device” refers to any appliance, usually mechanical or electrical, designed to perform a particular function.

The term “angiogenesis” refers to the process by which tumor cells trigger abnormal blood vessel growth to create their own blood supply. Angiogenesis is believed to be the mechanism via which tumors get needed nutrients to grow and metastasize to other locations in the body. Antiangiogenic agents interfere with these processes and destroy or control tumors. Angiogenesis an attractive therapeutic target for treating neoplastic disease because it is a multi-step process that occurs in a specific sequence, thus providing several possible targets for drug action. Examples of agents that interfere with several of these steps include compounds such as matrix metalloproteinase inhibitors (MMPIs) that block the actions of enzymes that clear and create paths for newly forming blood vessels to follow; compounds, such as avb₃ inhibitors, that interfere with molecules that blood vessel cells use to bridge between a parent blood vessel and a tumor; agents, such as COX-2 selective inhibiting agents, that prevent the growth of cells that form new blood vessels; and protein-based compounds that simultaneously interfere with several of these targets.

The phrase an “immunotherapeutic agent” refers to agents used to transfer the immunity of an immune donor, e.g., another person or an animal, to a host by inoculation. The term embraces the use of serum or gamma globulin containing performed antibodies produced by another individual or an animal; nonspecific systemic stimulation; adjuvants; active specific immunotherapy; and adoptive immunotherapy. Adoptive immunotherapy refers to the treatment of a disease by therapy or agents that include host inoculation of sensitized lymphocytes, transfer factor, immune RNA, or antibodies in serum or gamma globulin.

The phrase a “vaccine” includes agents that induce the patient's immune system to mount an immune response against the tumor by attacking cells that express tumor associated antigens (TAAs).

The phrase “antineoplastic agents” includes agents that exert antineoplastic effects, i.e., prevent the development, maturation, or spread of neoplastic cells, directly on the tumor cell, e.g., by cytostatic or cytocidal effects, and not indirectly through mechanisms such as biological response modification.

The present invention also provides a method for lowering the risk of a first or subsequent occurrence of a neoplastic disease event comprising the administration of a prophylactically effective amount of a combination of an aromatase inhibitor and a COX-2 inhibiting agent to a patient at risk for such a neoplastic disease event. The patient may already have non-malignant neoplastic disease at the time of administration, or be at risk for developing it.

Patients to be treated with the present combination therapy includes those at risk of developing neoplastic disease or of having a neoplastic disease event. Standard neoplastic disease risk factors are known to the average physician practicing in the relevant field of medicine. Such known risk factors include but are not limited to genetic factors and exposure to carcinogens such as certain viruses, certain chemicals, tobacco smoke or radiation. Patients who are identified as having one or more risk factors known in the art to be at risk of developing neoplastic disease, as well as people who already have neoplastic disease, are intended to be included within the group of people considered to be at risk for having a neoplastic disease event.

Studies indicate that prostaglandins synthesized by cyclooxygenases play a critical role in the initiation and promotion of cancer. Moreover, COX-2 is overexpressed in neoplastic lesions of the colon, breast, lung, prostate, esophagus, pancreas, intestine, cervix, ovaries, urinary bladder, and head and neck. Products of COX-2 activity, i.e., prostaglandins, stimulate proliferation, increase invasiveness of malignant cells, and enhance the production of vascular endothelial growth factor, which promotes angiogenesis. In several in vitro and animal models, COX-2 selective inhibiting agents have inhibited tumor growth and metastasis. The utility of COX-2 selective inhibiting agents as chemopreventive, antiangiogenic and chemotherapeutic agents is described in the literature, see for example Koki et al., Potential utility of COX-2 selective inhibiting agents in chemoprevention and chemotherapy. Exp. Opin. Invest. Drugs (1999) 8(10) pp. 1623-1638.

In addition to cancers per se, COX-2 is also expressed in the angiogenic vasculature within and adjacent to hyperplastic and neoplastic lesions indicating that COX-2. plays a role in angiogenesis. In both the mouse and rat, COX-2 selective inhibiting agents markedly inhibited bFGF-induced neovascularization.

Also, COX-2 levels are elevated in tumors with amplification and/or overexpression of other oncogenes including but not limited to c-myc, N-myc, L-myc, K-ras, H-ras, N-ras. Consequently, the administration of a COX-2 selective inhibiting agent and an aromatase inhibitor antineoplastic agent, in combination with an agent, or agents, that inhibits or suppresses oncogenes is contemplated to prevent or treat cancers in which oncogenes are overexpressed.

Accordingly, there is a need for a method of treating or preventing a cancer in a patient that overexpresses COX-2 or an oncogene.

Dosages, Formulations and Routes of Administration

### Dosages

Dosage levels of the source of a COX-2 inhibiting agent (e.g., a COX-2 selective inhibiting agent or a prodrug of a COX-2 selective inhibiting agent) on the order of about 0.1 mg to about 10,000 mg of the active ingredient compound are useful in the treatment of the above conditions, with preferred levels of about 1.0 mg to about 1,000 mg. While the dosage of active compound administered to a warm-blooded animal (a mammal), is dependent on the species of that mammal, the body weight, age, and individual condition, and on the routhe of administration, the unit dosage for oral administration to a mammal of about 50 to 70 kg may contain between about 5 and 500 mg of the active ingredient (for example, COX-189). The amount of active ingredient that may be combined with other anticancer agents to produce a single dosage form will vary depending upon the host treated and the particular mode of administration.

A total daily dose of an aromatase inhibitor can generally be in the range of from about 0.001 to about 10,000 mg/day in single or divided doses.

Table No. 9 provides illustrative examples of median dosages for selected aromatase inhibitors that may be used in combination with a COX-2 inhibitor. It should be noted that specific dose regimen for the chemotherapeutic agents below depends upon dosing considerations based upon a variety of factors including the type of neoplasia; the stage of the neoplasm; the age, weight, sex, and medical condition of the patient; the route of administration; the renal and hepatic function of the patient; and the particular combination employed.

TABLE No. 9
Median Dosages For Selected Aromatase Inhibitor
Antineoplastic Agents

| Aromatase Inhibitor | Median Dosage |
|---|---|
| Aminoglutethimide | 250 mg/day |
| Anastrozole | 1 mg/day |
| Exemestane | 25 mg/day |
| Fadrozole | 1 mg bid |
| Formestane | 250 mg/2 wk |
| Letrozole | 2.5 mg/day |
| Testolactone | 250 mg qid |
| Vorozole | 2.5 mg/day |

It is understood, however, that specific dose levels of the therapeutic agents or therapeutic approaches of the present invention for any particular patient depends upon a variety of factors including the activity of the specific compound employed, the age, body weight, general health, sex, and diet of the patient, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of cancers in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art.

### Formulations and Routes of Administration

Effective formulations and administration procedures are well known in the art and are described in standard textbooks.

The COX-2 inhibiting agent and the aromatase inhibitor antineoplastic agent can be formulated as a single pharmaceutical composition or as independent multiple pharmaceutical compositions. Pharmaceutical compositions according to the present invention include those suitable for oral, inhalation spray, rectal, topical, buccal (e.g., sublingual), or parenteral (e.g., subcutaneous, intramuscular, intravenous, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

Compounds and composition of the present invention can then be administered orally, by inhalation spray, rectally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

The compositions of the present invention can be administered for the treatment, prevention or inhibition of neoplastic disease or disorders by any means that produce contact of these compounds with their site of action in the body, for example in the ileum, the plasma, or the liver of a mammal.

Pharmaceutically acceptable salts are particularly suitable for medical applications because of their greater aqueous solubility relative to the parent compound. Such salts must clearly have a pharmaceutically acceptable anion or cation.

The compounds useful in the methods, combinations and compositions of the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well-known techniques of pharmacy, consisting essentially of admixing the components.

The amount of compound in combination that is required to achieve the desired biological effect will, of course, depend on a number of factors such as the specific compound chosen, the use for which it is intended, the mode of administration, and the clinical condition of the recipient.

The compounds of the present invention can be delivered orally either in a solid, in a semi-solid, or in a liquid form. Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of an aromatase inhibitor antineoplastic agent, the dose range may be from about 0.01 mg to about 5,000 mg or any other dose, dependent upon the specific inhibitor, as is known in the art. When in a liquid or in a semi-solid form, the combinations of the present invention can, for example, be in the form of a liquid, syrup, or contained in a gel capsule (e.g., a gel cap). In one embodiment, when an aromatase inhibitor antineoplastic agent is used in a combination of the present invention, the aromatase inhibitor antineoplastic agent can be provided in the form of a liquid, syrup, or contained in a gel capsule. In another embodiment, when a COX-2 inhibiting agent is used in a combination of the present invention, the COX-2 inhibiting agent can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Pharmaceutical compositions suitable for rectal administration are preferably presented as unit-dose suppositories. These can be prepared by admixing a compound or compounds of the present invention with one or more conventional solid carriers, for example, cocoa butter, synthetic mono- di- or triglycerides, fatty acids and polyethylene glycols that are solid at ordinary temperatures but liquid at the rectal temperature and will therefore melt in the rectum and release the drug; and then shaping the resulting mixture.

Pharmaceutical compositions suitable for topical application to the skin preferably take the form of an ointment, cream, lotion, paste, gel, spray, aerosol, or oil. Carriers which can be used include petroleum jelly (e.g., Vaseline), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research, 3(6), 318 (1986).

In any case, the amount of active ingredients that can be combined with carrier materials to produce a single dosage form to be administered will vary depending upon the host treated and the particular mode of administration.

In combination therapy, administration of two or more of the therapeutic agents useful in the methods, combinations and compositions of the present invention may take place sequentially in separate formulations, or may be accomplished by simultaneous administration in a single formulation or in a separate formulation. Independent administration of each therapeutic agent may be accomplished by, for example, oral, inhalation spray, rectal, topical, buccal (e.g., sublingual), or parenteral (e.g., subcutaneous, intramuscular, intravenous, intramedullary and intradermal injections, or infusion techniques) administration. The formulation may be in the form of a bolus, or in the form of aqueous or non-aqueous isotonic sterile injection solutions or suspensions. Solutions and suspensions may be prepared from sterile powders or granules having one or more pharmaceutically-acceptable carriers or diluents, or a binder such as gelatin or hydroxypropylmethyl cellulose, together with one or more of a lubricant, preservative, surface active or dispersing agent. The therapeutic compounds may further be administered by any combination of, for example, oral/oral, oral/parenteral, or parenteral/parenteral route.

The therapeutic compounds which make up the combination therapy may be a combined dosage form or in separate dosage forms intended for substantially simultaneous oral administration. The therapeutic compounds which make up the combination therapy may also be administered sequentially, with either therapeutic compound being administered by a regimen calling for two step ingestion. Thus, a regimen may call for sequential administration of the therapeutic compounds with spaced-apart ingestion of the separate, active agents. The time period between the multiple ingestion steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

### Treatment Regimen

Any effective treatment regimen can be utilized and readily determined and repeated as necessary to effect treatment. In clinical practice, the compositions containing a COX-2 inhibitor in combination with an aromatase inhibitor are administered in specific cycles until a response is obtained.

For patients who initially present without advanced or metastatic cancer, a COX-2 inhibitor based drug in combination with an aromatase inhibitor can be used as an immediate initial therapy prior to surgery, chemotherapy, or radiation therapy, and/or as a continuous post-treatment therapy in patients at risk for recurrence or metastasis (for example, in adenocarcinoma of the prostate, risk for metastasis is based upon high PSA, high Gleason's score, locally extensive disease, and/or pathological evidence of tumor invasion in the surgical specimen). The goal in these patients is to inhibit the growth of potentially metastatic cells from the primary tumor during surgery or radiotherapy and inhibit the growth of tumor cells from undetectable residual primary tumor.

For patients who initially present with advanced or metastatic cancer, a COX-2 inhibitor based drug in combination with an aromatase inhibitor is used as a continuous supplement to, or possible replacement for hormonal ablation. The goal in these patients is to slow or prevent tumor cell growth from both the untreated primary tumor and from the existing metastatic lesions.

In addition, the invention may be particularly efficacious during post-surgical recovery, where the present compositions and methods may be particularly effective in lessening the chances of recurrence of a tumor engendered by shed cells that cannot be removed by surgical intervention.

### Combinations with Other Treatments

The methods, combinations amd compositions of the present invention may be used in conjunction with other treatment modalities, including, but not limited to surgery and radiation, hormonal therapy, antiangiogenic therapy, chemotherapy, immunotherapy, and cryotherapy. The present invention may be used in conjunction with any current or future therapy.

The following discussion highlights some agents in this respect, which are illustrative, not limitative. A wide variety of other effective agents also may be used.

### Surgery and Radiation

In general, surgery and radiation therapy are employed as potentially curative therapies for patients under 70 years of age who present with clinically localized disease and are expected to live at least 10 years.

For example, approximately 70% of newly diagnosed prostate cancer patients fall into this category. Approximately 90% of these patients (65% of total patients) undergo surgery, while approximately 10% of these patients (7% of total patients) undergo radiation therapy. Histopathological examination of surgical specimens reveals that approximately 63% of patients undergoing surgery (40% of total patients) have locally extensive tumors or regional (lymph node) metastasis that was undetected at initial diagnosis. These patients are at a significantly greater risk of recurrence. Approximately 40% of these patients will actually develop recurrence within five years after surgery. Results after radiation are even less encouraging. Approximately 80% of patients who have undergone radiation as their primary therapy have disease persistence or develop recurrence or metastasis within five years after treatment. Currently, most of these surgical and radiotherapy patients generally do not receive any immediate follow-up therapy. Rather, for example, they are monitored frequently for elevated Prostate Specific Antigen (“PSA”), which is the primary indicator of recurrence or metastasis prostate cancer.

Thus, there is considerable opportunity to use the present invention in conjunction with surgical intervention.

### Hormonal Therapy

Hormonal ablation is the most effective palliative treatment for the 10% of patients presenting with metastatic prostate cancer at initial diagnosis. Hormonal ablation by medication and/or orchiectomy is used to block hormones that support the further growth and metastasis of prostate cancer. With time, both the primary and metastatic tumors of virtually all of these patients become hormone-independent and resistant to therapy. Approximately 50% of patients presenting with metastatic disease die within three years after initial diagnosis, and 75% of such patients die within five years after diagnosis. Continuous supplementation with NAALADase inhibitor based drugs are used to prevent or reverse this potentially metastasis-permissive state.

Among hormones which may be used in combination with the present inventive compounds, diethylstilbestrol (DES), leuprolide, flutamide, cyproterone acetate, ketoconazole and amino glutethimide are preferred.

### Immunotherapy

The combinations and methods of the present invention may also be used in combination with monoclonal antibodies in treating cancer. For example monoclonal antibodies may be used in treating prostate cancer. A specific example of such an antibody includes cell membrane-specific anti-prostate antibody.

The present invention may also be used with immunotherapies based on polyclonal or monoclonal antibody-derived reagents, for instance. Monoclonal antibody-based reagents are most preferred in this regard. Such reagents are well known to persons of ordinary skill in the art. Radiolabelled monoclonal antibodies for cancer therapy, such as the recently approved use of monoclonal antibody conjugated with strontium-89, also are well known to persons of ordinary skill in the art.

### Antiangiogenic Therapy

The combinations and methods of the present invention may also be used in combination with other antiangiogenic agents in treating cancer. Antiangiogenic agents include but are not limited to MMP inhibitors, integrin antagonists, COX-2 inhibitors, angiostatin, endostatin, thrombospondin-1, and interferon alpha. Examples of preferred antiangiogenic agents include, but are not limited to vitaxin, marimastat, Bay-12-9566, AG-3340, metastat, EMD-121974, and D-2163 (BMS-275291).

### Cryotherapy

Cryotherapy recently has been applied to the treatment of some cancers. Methods and compositions of the present invention also could be used in conjunction with an effective therapy of this type.

### Chemotherapy

There are large numbers of antineoplastic agents available in commercial use, in clinical evaluation and in pre-clinical development, which could be included in the present invention for treatment of neoplasia by combination drug chemotherapy. For convenience of

discussion, antineoplastic agents are classified into the following classes, subtypes and species:

ACE inhibitors,

alkylating agents,

angiogenesis inhibitors,

angiostatin,

anthracyclines/DNA intercalators,

anti-cancer antibiotics or antibiotic-type agents,

antimetabolites,

antimetastatic compounds,

asparaginases,

bisphosphonates,

cGMP phosphodiesterase inhibitors,

calcium carbonate,

cyclooxygenase-2 inhibitors

DHA derivatives,

DNA topoisomerase,

endostatin,

epipodophylotoxins,

genistein,

hormonal anticancer agents,

hydrophilic bile acids (URSO),

immunomodulators or immunological agents,

integrin antagonists

interferon antagonists or agents,

MMP inhibitors,

miscellaneous antineoplastic agents,

monoclonal antibodies,

nitrosoureas,

NSAIDs,

ornithine decarboxylase inhibitors,

pBATTs,

radio/chemo sensitizers/protectors,

retinoids

selective inhibitors of proliferation and migration of endothelial cells,

selenium,

stromelysin inhibitors,

taxanes,

vaccines, and

vinca alkaloids.

The major categories that some preferred antineoplastic agents fall into include antimetabolite agents, alkylating agents, antibiotic-type agents, hormonal anticancer agents, immunological agents, interferon-type agents, and a category of miscellaneous antineoplastic agents. Some antineoplastic agents operate through multiple or unknown mechanisms and can thus be classified into more than one category.

Therapeutic Illustrations

All of the various cell types of the body can be transformed into benign or malignant neoplasia or tumor cells and are contemplated as objects of the invention. A “benign” tumor cell denotes the non-invasive and non-metastasized state of a neoplasm. In man the most frequent neoplasia site is lung, followed by colorectal, breast, prostate, bladder, pancreas, and then ovary. Other prevalent types of cancer include leukemia, central nervous system cancers, including brain cancer, melanoma, lymphoma, erythroleukemia, uterine cancer, and head and neck cancer.

The following non-limiting illustrative examples describe various cancer diseases and therapeutic approaches that may be used in the present invention, and are for illustrative purposes only. Preferred COX-2 inhibitors of the below non-limiting illustrations include but are not limited to celecoxib, deracoxib, valdecoxib, chromene COX-2 inhibitors, parecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-d ifluorophenyl)-4-(3-hydroxy-3-methyl butoxy)-5-[4-(methylsulfonyl)phenyl]-3(2H)-pyridazinone, N-[2-(cyclohexyloxy)-4-n itrophenyl]methanesulfonamide, 2-[(2,4-dichloro-6-methylphenyl)amino]-5-ethyl-benzeneacetic acid, diarylmethylidenefuran derivative COX-2 inhibitors, and BMS 347070 or other similar compounds.

Preferred aromatase inhibitors of the below non-limiting illustrations include but are not limited to aminoglutethimide, anastrozole, atamestane, exemestane, fadrozole, finrozole, formestane, letrozole, testolactone and 4-[[(4-bromophenyl)methyl]-4H-1,2,4-triazol-4-ylamino]benzonitrile.

### Illustration 1: Lung Cancer

In many countries including Japan, Europe and America, the number of patients with lung cancer is fairly large and continues to increase year after year and is the most frequent cause of cancer death in both men and women. Although there are many potential causes for lung cancer, tobacco use, and particularly cigarette smoking, is the most important. Additionally, etiologic factors such as exposure to asbestos, especially in smokers, or radon are contributory factors. Also occupational hazards such as exposure to uranium have been identified as an important factor. Finally, genetic factors have also been identified as another factor that increase the risk of cancer.

Lung cancers can be histologically classified into non-small cell lung cancers (e.g. squamous cell carcinoma (epidermoid), adenocarcinoma, large cell carcinoma (large cell anaplastic), etc.) and small cell lung cancer (oat cell). Non-small cell lung cancer (NSCLC) has different biological properties and responses to chemotherapeutics from those of small cell lung cancer (SCLC). Thus, chemotherapeutic formulas and radiation therapy are different between these two types of lung cancer.

Non-Small Cell Lung Cancer

In the present invention, a preferred therapy for the treatment of NSCLC is a combination of neoplasia disorder effective amounts of a COX-2 inhibitor and an aromatase inhibitor in combination with one or more of the following combinations of antineoplastic agents: 1) ifosfamide, cisplatin, etoposide; 2) cyclophosphamide, doxorubicin, cisplatin; 3) ifosfamide, carboplatin, etoposide; 4) bleomycin, etoposide, cisplatin; 5) ifosfamide, mitomycin, cisplatin; 6) cisplatin, vinblastine; 7) cisplatin, vindesine; 8) mitomycin C, vinblastine, cisplatin; 9) mitomycin C, vindesine, cisplatin; 10) ifosfamide, etoposide; 11) etoposide, cisplatin; 12) ifosfamide, mitomycin C; 13) flurouracil, cisplatin, vinblastine; 14) carboplatin, etoposide; or radiation therapy.

In the present invention, a further preferred therapy for the treatment of NSCLC is a composition of a neoplasia disorder effective amounts of a COX-2 selective inhibitor in combination with an aromatase inhibitor.

Small Cell Lung Cancer

In another embodiment of the present invention, a preferred therapy for the treatment of small cell lung cancer is a combination of neoplasia disorder effective amounts of a COX-2 inhibitor in combination with an aromatase inhibitor.

Additionally, radiation therapy in conjunction with the preferred combinations of COX-2 inhibitors and aromatase inhibitors is contemplated to be effective at increasing the response rate for SCLC patients. The typical dosage regimen for radiation therapy ranges from 40 to 55 Gy, in 15 to 30 fractions, 3 to 7 times week. The tissue volume to be irradiated will be determined by several factors and generally the hilum and subcarnial nodes, and bialteral mdiastinal nodes up to the thoraic inlet are treated, as well as the primary tumor up to 1.5 to 2.0 cm of the margins.

A preferred therapeutic combination for the treatment of small cell lung cancer in the present invention is a combination of celecoxib and exemestane.

### Illustration 2: Colorectal Cancer

Tumor metastasis prior to surgery is generally believed to be the cause of surgical intervention failure and up to one year of chemotherapy is required to kill the non-excised tumor cells. Because severe toxicity is associated with the chemotherapeutic agents, only patients at high risk of recurrence are placed on chemotherapy following surgery. Thus, the incorporation of a COX-2 inhibitor and an aromatase inhibitor into the management of colorectal cancer will play an important role in the treatment of colorectal cancer and lead to overall improved survival rates for patients diagnosed with colorectal cancer.

In one embodiment of the present invention, a combination therapy for the treatment of colorectal cancer is surgery, followed by a regimen of a COX-2 inhibiting agent and an aromatase inhibitor, cycled over a one year time period. In another embodiment, a combination therapy for the treatment of colorectal cancer is a regimen of a COX-2 inhibiting agent and an aromatase inhibitor, followed by surgical removal of the tumor from the colon or rectum and then followed be a regimen of a COX-2 inhibiting agent and an aromatase inhibitor, cycled over a one year time period. In still another embodiment, a therapy for the treatment of colon cancer is a combination of neoplasia disorder effective amounts of a COX-2 inhibiting agent and an aromatase inhibitor.

A preferred therapeutic combination in the present invention for the treatment of colorectal cancer is a combination of celecoxib and exemestane.

### Illustration 3: Breast Cancer

In the treatment of locally advanced noninflammatory breast cancer, a COX-2 inhibitor and an aromatase inhibitor will be useful to treat the disease in combination with surgery, radiation therapy and/or chemotherapy. Preferred combinations of chemotherapeutic agents, radiation therapy and surgery that can be used in combination with the present invention include, but are not limited to the following combinations: 1) doxorubicin, vincristine, radical mastectomy; 2) doxorubicin, vincristine, radiation therapy; 3) cyclophosphamide, doxorubicin, 5-flourouracil, vincristine, prednisone, mastecomy; 4) cyclophosphamide, doxorubicin, 5-flourouracil, vincristine, prednisone, radiation therapy; 5) cyclophosphamide, doxorubicin, 5-flourouracil, premarin, tamoxifen, radiation therapy for pathologic complete response; 6) cyclophosphamide, doxorubicin, 5-flourouracil, premarin, tamoxifen, mastectomy, radiation therapy for pathologic partial response; 7) mastectomy, radiation therapy, levamisole; 8) mastectomy, radiation therapy; 9) mastectomy, vincristine, doxorubicin, cyclophosphamide, levamisole; 10) mastectomy, vincristine, doxorubicin, cyclophosphamide; 11) mastecomy, cyclophosphamide, doxorubicin, 5-fluorouracil, tamoxifen, halotestin, radiation therapy; 12) mastecomy, cyclophosphamide, doxorubicin, 5-fluorouracil, tamoxifen, halotestin.

In the treatment of locally advanced inflammatory breast cancer, a COX-2 inhibitor and an aromatase inhibitor can be used to treat the disease in combination with surgery, radiation therapy or with chemotherapeutic agents. In one embodiment, combinations of chemotherapeutic agents, radiation therapy and surgery that can be used in combination with the present invention include, but or not limited to the following combinations: 1) cyclophosphamide, doxorubicin, 5-fluorouracil, radiation therapy; 2) cyclophosphamide, doxorubicin, 5-fluorouracil, mastectomy, radiation therapy; 3) 5-flurouracil, doxorubicin, clyclophosphamide, vincristine, prednisone, mastectomy, radiation therapy; 4) 5-flurouracil, doxorubicin, clyclophosphamide, vincristine, mastectomy, radiation therapy; 5) cyclophosphamide, doxorubicin, 5-fluorouracil, vincristine, radiation therapy; 6) cyclophosphamide, doxorubicin, 5-fluorouracil, vincristine, mastectomy, radiation therapy; 7) doxorubicin, vincristine, methotrexate, radiation therapy, followed by vincristine, cyclophosphamide, 5-florouracil; 8) doxorubicin, vincristine, cyclophosphamide, methotrexate, 5-florouracil, radiation therapy, followed by vincristine, cyclophosphamide, 5-florouracil; 9) surgery, followed by cyclophosphamide, methotrexate, 5-fluorouracil, prednisone, tamoxifen, followed by radiation therapy, followed by cyclophosphamide, methotrexate, 5-fluorouracil, predinsone, tamoxifen, doxorubicin, vincristine, tamoxifen; 10) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine, tamoxifen; 11) surgery, followed by cyclophosphamide, methotrexate, 5-fluorouracil, predinsone, tamoxifen, followed by radiation therapy, followed by cyclophosphamide, methotrexate, 5-fluorouracil, doxorubicin, vincristine, tamoxifen; 12) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine; 13) surgery, followed by cyclophosphamide, methotrexate, 5-fluorouracil, predinsone, tamoxifen, followed by radiation therapy, followed by cyclophosphamide, methotrexate, 5-fluorouracil, predinsone, tamoxifen, doxorubicin, vincristine, tamoxifen; 14) surgery, followed by cyclophosphamide, methotrexate, 5-fluorouracil, followed by radiation therapy, followed by cyclophosphamide, methotrexate, 5-fluorouracil, predinsone, tamoxifen, doxorubicin, vincristine; 15) surgery, followed by cyclophosphamide, methotrexate, 5-fluorouracil, predinsone, tamoxifen, followed by radiation therapy, followed by cyclophosphamide, methotrexate, 5-fluorouracil, doxorubicin, vincristine; 16) 5-florouracil, doxorubicin, cyclophosphamide followed by mastectomy, followed by 5-florouracil, doxorubicin, cyclophosphamide, followed by radtiation therapy.

In the treatment of metastatic breast cancer, a COX-2 inhibitor and an aromatase inhibitor can be used to treat the disease in combination with surgery, radiation therapy or with chemotherapeutic agents. In one embodiment, combinations of chemotherapeutic agents that can be used in combination with a COX-2 inhibitor and an aromatase inhibitor of the present invention include, but are not limited to the following combinations: 1) cyclophosphamide, methotrexate, 5-fluorouracil; 2) cyclophosphamide, adriamycin, 5-fluorouracil; 3) cyclophosphamide, methotrexate, 5-flurouracil, vincristine, prednisone; 4) adriamycin, vincristine; 5) thiotepa, adriamycin, vinblastine; 6) mitomycin, vinblastine; 7) cisplatin, etoposide.

A preferred therapeutic combination for the treatment of breast cancer in the present invention is a combination of celecoxib and exemestane.

A further preferred therapeutic combination of the present invention for the treatment of breast cancer is a combination of celecoxib, exemestane and tamoxifen.

### EXAMPLE 4

### Prostate Cancer

U.S. Pat. No. 4,596,797 discloses aromatase inhibitors as a method of prophylaxis and/or treatment of prostatic hyperplasia.

In one embodiment of the present invention, a therapy for the treatment of prostate cancer is a combination of amounts of a COX-2 selective inhibitor and an aromatase inhibitor which together comprise a therapeutically effective amount.

A preferred therapeutic combination for the treatment of prostate cancer is a combination of celecoxib and exemestane.

Illustration 5: Bladder Cancer

The classification of bladder cancer is divided into three main classes: 1) superficial disease, 2) muscle-invasive disease, and 3) metastatic disease.

Currently, transurethral resection (TUR), or segmental resection, account for first line therapy of superficial bladder cancer, i.e., disease confined to the mucosa or the lamina propria. However, intravesical therapies are necessary, for example, for the treatment of high-grade tumors, carcinoma in situ, incomplete resections, recurrences, and multifocal papillary. Recurrence rates range from up to 30 to 80 percent, depending on stage of cancer.

Therapies that are currently used as intravesical therapies include chemotherapy, immunotherapy, bacille Calmette-Guerin (BCG) and photodynamic therapy. The main objective of intravesical therapy is twofold: to prevent recurrence in high-risk patients and to treat disease that cannot by resected. The use of intravesical therapies must be balanced with its potentially toxic side effects. Additionally, BCG requires an unimpaired immune system to induce an antitumor effect. Chemotherapeutic agents that are known to be inactive against superficial bladder cancer include Cisplatin, actinomycin D, 5-fluorouracil, bleomycin, and cyclophosphamide methotrexate.

In the treatment of superficial bladder cancer, a COX-2 inhibitor can be used to treat the disease in combination with an aromatase inhibitor, or in combination with surgery (TUR), other chemotherapy and intravesical therapies.

In one embodiment, an intravesicle immunotherapeutic agent that may be used in the present invention is BCG. A preferred daily dose ranges from 60 to 120 mg, depending on the strain of the live attenuated tuberculosis organism used.

In another embodiment, a photodynamic therapeutic agent that may be used with the present invention is Photofrin I, a photosensitizing agent, administered intravenously. It is taken up by the low-density lipoprotein receptors of the tumor cells and is activated by exposure to visible light. Additionally, neomydium YAG laser activation generates large amounts of cytotoxic free radicals and singlet oxygen.

In the treatment of muscle-invasive bladder cancer, a COX-2 inhibitor and an aromatase inhibitor can be used to treat the disease in combination with surgery (TUR), intravesical chemotherapy, radiation therapy, and radical cystectomy with pelvic lymph node dissection.

In one embodiment, the radiation dose for the treatment of bladder cancer is between 5,000 to 7,000 cGY in fractions of 180 to 200 cGY to the tumor. Additionally, a 3,500 to 4,700 cGY total dose is administered to the normal bladder and pelvic contents in a four-field technique. Radiation therapy should be considered only if the patient is not a surgical candidate, but may be considered as preoperative therapy.

Currently no curative therapy exists for metastatic bladder cancer. The present invention contemplates an effective treatment of bladder cancer leading to improved tumor inhibition or regression, as compared to current therapies. In one embodiment for the treatment of metastatic bladder cancer, a COX-2 inhibitor and an aromatase inhibitor will be useful to treat the disease, optionally in combination with surgery, radiation therapy or with chemotherapeutic agents.

A preferred therapeutic combination of the present invention for the treatment of bladder cancer is a combination of celecoxib and exemestane.

### Illustration 6: Pancreas Cancer

Approximately 2% of new cancer cases diagnosed in the United States are pancreatic cancer. Pancreatic cancer is generally classified into two clinical types: 1) adenocarcinoma (metastatic and non-metastatic), and 2) cystic neoplasms (serous cystadenomas, mucinous cystic neoplasms, papilary cystic neoplasms, acinar cell systadenocarcinoma, cystic choriocarcinoma, cystic teratomas, angiomatous neoplasms).

In one embodiment, a therapy for the treatment of non-metastatic adenocarcinoma that may be used in the present invention includes the use of a COX-2 inhibitor and an aromatase inhibitor along with preoperative bilary tract decompression (patients presenting with obstructive jaundice); surgical resection, including standard resection, extended or radial resection and distal pancreatectomy (tumors of body and tail); adjuvant radiation; antiangiogenic therapy; and chemotherapy.

In another embodiment for the treatment of metastatic adenocarcinoma, a therapy of the present invention comprises a COX-2 inhibitor and an aromatase inhibitor in combination with continuous treatment of 5-fluorouracil, followed by weekly cisplatin therapy.

In yet another embodiment, a combination therapy for the treatment of cystic neoplasms is the use of a COX-2 inhibitor and an aromatase inhibitor along with resection.

A preferred therapeutic combination of the present invention for the treatment of pancreatic cancer is a combination of celecoxib and exemestane.

### Illustration 7: Ovary Cancer

Celomic epithelial carcinoma accounts for approximately 90% of ovarian cancer cases. In one embodiment, a therapy for the treatment of ovary cancer is a combination of therapeutically effective amounts of a COX-2 inhibitor and an aromatase inhibitor.

In another embodiment, a method for the treatment of celomic epithelial—carcinoma is a combination of therapeutically effective amounts of a COX-2 inhibitor and an aromatase inhibitor in combination with the following combinations of antineoplastic agents: 1) cisplatin, doxorubicin, cyclophosphamide; 2) hexamethylmelamine, cyclosphamide, doxorubicin, cisplatin; 3) cyclophosphamide, hexamethylmelamine, 5-flurouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethlmelamine, doxorubicin, cisplatin; 12) carboplatin, cyclophosphamide; 13) cisplatin, cyclophosphamide.

Germ cell ovarian cancer accounts for approximately 5% of ovarian cancer cases. Germ cell ovarian carcinomas are classified into two main groups: 1) dysgerminoma, and nondysgerminoma. Nondysgerminoma is further classified into teratoma, endodermal sinus tumor, embryonal carcinoma, chloricarcinoma, polyembryoma, and mixed cell tumors.

In one embodiment of the present invention, a therapy for the treatment of germ cell carcinoma is a combination of therapeutically effective amounts of a COX-2 inhibitor and an aromatase inhibitor.

In another embodiment of the present invention, a therapy for the treatment of germ cell carcinoma is a combination of therapeutically effective amounts of a COX-2 inhibitor and an aromatase inhibitor in combination with the following combinations of antineoplastic agents: 1) vincristine, actinomycin D, cyclophosphamide; 2) bleomycin, etoposide, cisplatin; 3) vinblastine, bleomycin, cisplatin.

Cancer of the fallopian tube is the least common type of ovarian cancer, accounting for approximately 400 new cancer cases per year in the United States. Papillary serous adenocarcinoma accounts for approximately 90% of all malignancies of the ovarian tube.

In one embodiment of the present invention, a therapy for the treatment of fallopian tube cancer is a combination of neoplasia disorder effective amounts of a COX-2 inhibiting agent and an aromatase inhibitor.

Another embodiment of the present invention for the treatment of fallopian tube cancer is a combination of therapeutically effective amounts of a COX-2 inhibitor and an aromatase inhibitor in combination with the following combinations of antineoplastic agents: 1) cisplatin, doxorubicin, cyclophosphamide; 2) hexamthylmelamine, cyclosphamide, doxorubicin, cisplatin; 3) cyclophosphamide, hexamehtylmelamine, 5-flurouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethimelamine, doxorubicin, cisplatin; 12) carboplatin, cyclophosphamide; 13) cisplatin, cyclophosphamide.

A preferred therapeutic combination for the treatment of ovarian cancer is a combination of celecoxib and exemestane.

### Illustration 8: Central Nervous System Cancers

Central nervous system cancer accounts for approximately 2% of new cancer cases in the United States. Common intracranial neoplasms include glioma, meninigioma, neurinoma, and adenoma.

In one embodiment of the present invention, a therapy for the treatment of central nervous system cancers is a combination of therapeutically effective amounts of a COX-2 inhibitor and an aromatase inhibitor.

In another embodiment of the present invention, a therapy for the treatment of maligant glioma is a combination of therapeutically effective amounts of a COX-2 inhibitor and an aromatase inhibitor in combination with the following combinations of therapies and antineoplastic agents: 1) radiation therapy, BCNU (carmustine); 2) radiation therapy, methyl CCNU (lomustine); 3) radiation therapy, medol; 4) radiation therapy, procarbazine; 5) radiation therapy, BCNU, medrol; 6) hyperfraction radiation therapy, BCNU; 7) radiation therapy, misonidazole, BCNU; 8) radiation therapy, streptozotocin; 9) radiation therapy, BCNU, procarbazine; 10) radiation therapy, BCNU, hydroxyurea, procarbazine, VM-26; 11) radiation therapy, BNCU, 5-flourouacil; 12) radiation therapy, Methyl CCNU, dacarbazine; 13) radiation therapy, misonidazole, BCNU; 14) diaziquone; 15) radiation therapy, PCNU; 16) procarbazine (matulane), CCNU, vincristine. A preferred dose of radiation therapy is about 5,500 to about 6,000 cGY. Preferred radiosensitizers include misonidazole, intra-arterial Budr and intravenous iododeoxyuridine (IUdR). It is also contemplated that radiosurgery may be used in combinations with antiangiogenesis agents.

A preferred therapeutic combination of the present invention for the treatment of central nervous system cancers is a combination of celecoxib and exemestane.

### Illustration 9

Additional examples of combinations are listed in Table No. 10.

TABLE No. 10
Combination therapy examples

| COX-2 | Antineoplastic | |
|---|---|---|
| Inhibitor | Agents | Indication |
| Celecoxib | Anastrozole | Breast |
| Celecoxib | Letrozole | Breast |
| Celecoxib | Exemestane | Breast |
| Rofecoxib | Anastrozole | Breast |
| Rofecoxib | Letrozole | Breast |
| Rofecoxib | Exemestane | Breast |
| JTE-522 | Anastrozole | Breast |
| JTE-522 | Letrozole | Breast |
| JTE-522 | Exemestane | Breast |
| Valdecoxib | Anastrozole | Breast |
| Valdecoxib | Letrozole | Breast |
| Valdecoxib | Exemestane | Breast |
| Parecoxib | Anastrozole | Breast |
| Parecoxib | Letrozole | Breast |
| Parecoxib | Exemestane | Breast |
| Etoricoxib | Anastrozole | Breast |
| Etoricoxib | Letrozole | Breast |
| Etoricoxib | Exemestane | Breast |

### Illustration 10

Table 11 illustrates examples of some combinations of the present invention wherein the combination comprises an amount of a COX-2 selective inhibitor source and an amount of an aromatase inhibitor wherein the amounts together comprise a neoplasia disorder effective amount of the compounds.

TABLE No. 11
Combinations of COX-2 selective inhibiting agents and
aromatase inhibitors

| Example | | Aromatase |
|---|---|---|
| Number | COX-2 Inhibitor | Inhibitor |
| 1 | C1 | A1 |
| 2 | C1 | A2 |
| 3 | C1 | A3 |
| 4 | C1 | A4 |
| 5 | C1 | A5 |
| 6 | C1 | A6 |
| 7 | C1 | A7 |
| 8 | C1 | A8 |
| 9 | C1 | A9 |
| 10 | C1 | A10 |
| 11 | C1 | A11 |
| 12 | C1 | A12 |
| 13 | C1 | A13 |
| 14 | C1 | A14 |
| 15 | C1 | A15 |
| 16 | C1 | A16 |
| 17 | C1 | A17 |
| 18 | C1 | A18 |
| 19 | C1 | A19 |
| 20 | C1 | A20 |
| 21 | C1 | A21 |
| 22 | C1 | A22 |
| 23 | C1 | A23 |
| 24 | C1 | A24 |
| 25 | C1 | A25 |
| 26 | C1 | A26 |
| 27 | C1 | A27 |
| 28 | C1 | A28 |
| 29 | C1 | A29 |
| 30 | C1 | A30 |
| 31 | C1 | A31 |
| 32 | C1 | A32 |
| 33 | C2 | A1 |
| 34 | C2 | A2 |
| 35 | C2 | A3 |
| 36 | C2 | A4 |
| 37 | C2 | A5 |
| 38 | C2 | A6 |
| 39 | C2 | A7 |
| 40 | C2 | A8 |
| 41 | C2 | A9 |
| 42 | C2 | A10 |
| 43 | C2 | A11 |
| 44 | C2 | A12 |
| 45 | C2 | A13 |
| 46 | C2 | A14 |
| 47 | C2 | A15 |
| 48 | C2 | A16 |
| 49 | C2 | A17 |
| 50 | C2 | A18 |
| 51 | C2 | A19 |
| 52 | C2 | A20 |
| 53 | C2 | A21 |
| 54 | C2 | A22 |
| 55 | C2 | A23 |
| 56 | C2 | A24 |
| 57 | C2 | A25 |
| 58 | C2 | A26 |
| 59 | C2 | A27 |
| 60 | C2 | A28 |
| 61 | C2 | A29 |
| 62 | C2 | A30 |
| 63 | C2 | A31 |
| 64 | C2 | A32 |
| 65 | C3 | A1 |
| 66 | C3 | A2 |
| 67 | C3 | A3 |
| 68 | C3 | A4 |
| 69 | C3 | A5 |
| 70 | C3 | A6 |
| 71 | C3 | A7 |
| 72 | C3 | A8 |
| 73 | C3 | A9 |
| 74 | C3 | A10 |
| 75 | C3 | A11 |
| 76 | C3 | A12 |
| 77 | C3 | A13 |
| 78 | C3 | A14 |
| 79 | C3 | A15 |
| 80 | C3 | A16 |
| 81 | C3 | A17 |
| 82 | C3 | A18 |
| 83 | C3 | A19 |
| 84 | C3 | A20 |
| 85 | C3 | A21 |
| 86 | C3 | A22 |
| 87 | C3 | A23 |
| 88 | C3 | A24 |
| 89 | C3 | A25 |
| 90 | C3 | A26 |
| 91 | C3 | A27 |
| 92 | C3 | A28 |
| 93 | C3 | A29 |
| 94 | C3 | A30 |
| 95 | C3 | A31 |
| 96 | C3 | A32 |
| 97 | C4 | A1 |
| 98 | C4 | A2 |
| 99 | C4 | A3 |
| 100 | C4 | A4 |
| 101 | C4 | A5 |
| 102 | C4 | A6 |
| 103 | C4 | A7 |
| 104 | C4 | A8 |
| 105 | C4 | A9 |
| 106 | C4 | A10 |
| 107 | C4 | A11 |
| 108 | C4 | A12 |
| 109 | C4 | A13 |
| 110 | C4 | A14 |
| 111 | C4 | A15 |
| 112 | C4 | A16 |
| 113 | C4 | A17 |
| 114 | C4 | A18 |
| 115 | C4 | A19 |
| 116 | C4 | A20 |
| 117 | C4 | A21 |
| 118 | C4 | A22 |
| 119 | C4 | A23 |
| 120 | C4 | A24 |
| 121 | C4 | A25 |
| 122 | C4 | A26 |
| 123 | C4 | A27 |
| 124 | C4 | A28 |
| 125 | C4 | A29 |
| 126 | C4 | A30 |
| 127 | C4 | A31 |
| 128 | C4 | A32 |
| 129 | C5 | A1 |
| 130 | C5 | A2 |
| 131 | C5 | A3 |
| 132 | C5 | A4 |
| 133 | C5 | A5 |
| 134 | C5 | A6 |
| 135 | C5 | A7 |
| 136 | C5 | A8 |
| 137 | C5 | A9 |
| 138 | C5 | A10 |
| 139 | C5 | A11 |
| 140 | C5 | A12 |
| 141 | C5 | A13 |
| 142 | C5 | A14 |
| 143 | C5 | A15 |
| 144 | C5 | A16 |
| 145 | C5 | A17 |
| 146 | C5 | A18 |
| 147 | C5 | A19 |
| 148 | C5 | A20 |
| 149 | C5 | A21 |
| 150 | C5 | A22 |
| 151 | C5 | A23 |
| 152 | C5 | A24 |
| 153 | C5 | A25 |
| 154 | C5 | A26 |
| 155 | C5 | A27 |
| 156 | C5 | A28 |
| 157 | C5 | A29 |
| 158 | C5 | A30 |
| 159 | C5 | A31 |
| 160 | C5 | A32 |
| 161 | C6 | A1 |
| 162 | C6 | A2 |
| 163 | C6 | A3 |
| 164 | C6 | A4 |
| 165 | C6 | A5 |
| 166 | C6 | A6 |
| 167 | C6 | A7 |
| 168 | C6 | A8 |
| 169 | C6 | A9 |
| 170 | C6 | A10 |
| 171 | C6 | A11 |
| 172 | C6 | A12 |
| 173 | C6 | A13 |
| 174 | C6 | A14 |
| 175 | C6 | A15 |
| 176 | C6 | A16 |
| 177 | C6 | A17 |
| 178 | C6 | A18 |
| 179 | C6 | A19 |
| 180 | C6 | A20 |
| 181 | C6 | A21 |
| 182 | C6 | A22 |
| 183 | C6 | A23 |
| 184 | C6 | A24 |
| 185 | C6 | A25 |
| 186 | C6 | A26 |
| 187 | C6 | A27 |
| 188 | C6 | A28 |
| 189 | C6 | A29 |
| 190 | C6 | A30 |
| 191 | C6 | A31 |
| 192 | C6 | A32 |
| 193 | C7 | A1 |
| 194 | C7 | A2 |
| 195 | C7 | A3 |
| 196 | C7 | A4 |
| 197 | C7 | A5 |
| 198 | C7 | A6 |
| 199 | C7 | A7 |
| 200 | C7 | A8 |
| 201 | C7 | A9 |
| 202 | C7 | A10 |
| 203 | C7 | A11 |
| 204 | C7 | A12 |
| 205 | C7 | A13 |
| 206 | C7 | A14 |
| 207 | C7 | A15 |
| 208 | C7 | A16 |
| 209 | C7 | A17 |
| 210 | C7 | A18 |
| 211 | C7 | A19 |
| 212 | C7 | A20 |
| 213 | C7 | A21 |
| 214 | C7 | A22 |
| 215 | C7 | A23 |
| 216 | C7 | A24 |
| 217 | C7 | A25 |
| 218 | C7 | A26 |
| 219 | C7 | A27 |
| 220 | C7 | A28 |
| 221 | C7 | A29 |
| 222 | C7 | A30 |
| 223 | C7 | A31 |
| 224 | C7 | A32 |
| 225 | C23 | A1 |
| 226 | C23 | A2 |
| 227 | C23 | A3 |
| 228 | C23 | A4 |
| 229 | C23 | A5 |
| 230 | C23 | A6 |
| 231 | C23 | A7 |
| 232 | C23 | A8 |
| 233 | C23 | A9 |
| 234 | C23 | A10 |
| 235 | C23 | A11 |
| 236 | C23 | A12 |
| 237 | C23 | A13 |
| 238 | C23 | A14 |
| 239 | C23 | A15 |
| 240 | C23 | A16 |
| 241 | C23 | A17 |
| 242 | C23 | A18 |
| 243 | C23 | A19 |
| 244 | C23 | A20 |
| 245 | C23 | A21 |
| 246 | C23 | A22 |
| 247 | C23 | A23 |
| 248 | C23 | A24 |
| 249 | C23 | A25 |
| 250 | C23 | A26 |
| 251 | C23 | A27 |
| 252 | C23 | A28 |
| 253 | C23 | A29 |
| 254 | C23 | A30 |
| 255 | C23 | A31 |
| 256 | C23 | A32 |
| 257 | C44 | A1 |
| 258 | C44 | A2 |
| 259 | C44 | A3 |
| 260 | C44 | A4 |
| 261 | C44 | A5 |
| 262 | C44 | A6 |
| 263 | C44 | A7 |
| 264 | C44 | A8 |
| 265 | C44 | A9 |
| 266 | C44 | A10 |
| 267 | C44 | A11 |
| 268 | C44 | A12 |
| 269 | C44 | A13 |
| 270 | C44 | A14 |
| 271 | C44 | A15 |
| 272 | C44 | A16 |
| 273 | C44 | A17 |
| 274 | C44 | A18 |
| 275 | C44 | A19 |
| 276 | C44 | A20 |
| 277 | C44 | A21 |
| 278 | C44 | A22 |
| 279 | C44 | A23 |
| 280 | C44 | A24 |
| 281 | C44 | A25 |
| 282 | C44 | A26 |
| 283 | C44 | A27 |
| 284 | C44 | A28 |
| 285 | C44 | A29 |
| 286 | C44 | A30 |
| 287 | C44 | A31 |
| 288 | C44 | A32 |
| 289 | C46 | A1 |
| 290 | C46 | A2 |
| 291 | C46 | A3 |
| 292 | C46 | A4 |
| 293 | C46 | A5 |
| 294 | C46 | A6 |
| 295 | C46 | A7 |
| 296 | C46 | A8 |
| 297 | C46 | A9 |
| 298 | C46 | A10 |
| 299 | C46 | A11 |
| 300 | C46 | A12 |
| 301 | C46 | A13 |
| 302 | C46 | A14 |
| 303 | C46 | A15 |
| 304 | C46 | A16 |
| 305 | C46 | A17 |
| 306 | C46 | A18 |
| 307 | C46 | A19 |
| 308 | C46 | A20 |
| 309 | C46 | A21 |
| 310 | C46 | A22 |
| 311 | C46 | A23 |
| 312 | C46 | A24 |
| 313 | C46 | A25 |
| 314 | C46 | A26 |
| 315 | C46 | A27 |
| 316 | C46 | A28 |
| 317 | C46 | A29 |
| 318 | C46 | A30 |
| 319 | C46 | A31 |
| 320 | C46 | A32 |
| 321 | C66 | A1 |
| 322 | C66 | A2 |
| 323 | C66 | A3 |
| 324 | C66 | A4 |
| 325 | C66 | A5 |
| 326 | C66 | A6 |
| 327 | C66 | A7 |
| 328 | C66 | A8 |
| 329 | C66 | A9 |
| 330 | C66 | A10 |
| 331 | C66 | A11 |
| 332 | C66 | A12 |
| 333 | C66 | A13 |
| 334 | C66 | A14 |
| 335 | C66 | A15 |
| 336 | C66 | A16 |
| 337 | C66 | A17 |
| 338 | C66 | A18 |
| 339 | C66 | A19 |
| 340 | C66 | A20 |
| 341 | C66 | A21 |
| 342 | C66 | A22 |
| 343 | C66 | A23 |
| 344 | C66 | A24 |
| 345 | C66 | A25 |
| 346 | C66 | A26 |
| 347 | C66 | A27 |
| 348 | C66 | A28 |
| 349 | C66 | A29 |
| 350 | C66 | A30 |
| 351 | C66 | A31 |
| 352 | C66 | A32 |
| 353 | C67 | A1 |
| 354 | C67 | A2 |
| 355 | C67 | A3 |
| 356 | C67 | A4 |
| 357 | C67 | A5 |
| 358 | C67 | A6 |
| 359 | C67 | A7 |
| 360 | C67 | A8 |
| 361 | C67 | A9 |
| 362 | C67 | A10 |
| 363 | C67 | A11 |
| 364 | C67 | A12 |
| 365 | C67 | A13 |
| 366 | C67 | A14 |
| 367 | C67 | A15 |
| 368 | C67 | A16 |
| 369 | C67 | A17 |
| 370 | C67 | A18 |
| 371 | C67 | A19 |
| 372 | C67 | A20 |
| 373 | C67 | A21 |
| 374 | C67 | A22 |
| 375 | C67 | A23 |
| 376 | C67 | A24 |
| 377 | C67 | A25 |
| 318 | C67 | A26 |
| 379 | C67 | A27 |
| 380 | C67 | A28 |
| 381 | C67 | A29 |
| 382 | C67 | A30 |
| 383 | C67 | A31 |
| 384 | C67 | A32 |
| 385 | a chromene COX-2 | A1 |
| | inhibitor | |
| 386 | a chromene COX-2 | A2 |
| | inhibitor | |
| 387 | a chromene COX-2 | A3 |
| | inhibitor | |
| 388 | a chromene COX-2 | A4 |
| | inhibitor | |
| 389 | a chromene COX-2 | A5 |
| | inhibitor | |
| 390 | a chromene COX-2 | A6 |
| | inhibitor | |
| 391 | a chromene COX-2 | A7 |
| | inhibitor | |
| 392 | a chromene COX-2 | A8 |
| | inhibitor | |
| 393 | a chromene COX-2 | A9 |
| | inhibitor | |
| 394 | a chromene COX-2 | A10 |
| | inhibitor | |
| 395 | a chromene COX-2 | A11 |
| | inhibitor | |
| 396 | a chromene COX-2 | A12 |
| | inhibitor | |
| 397 | a chromene COX-2 | A13 |
| | inhibitor | |
| 398 | a chromene COX-2 | A14 |
| | inhibitor | |
| 399 | a chromene COX-2 | A15 |
| | inhibitor | |
| 400 | a chromene COX-2 | A16 |
| | inhibitor | |
| 401 | a chromene COX-2 | A17 |
| | inhibitor | |
| 402 | a chromene COX-2 | A18 |
| | inhibitor | |
| 403 | a chromene COX-2 | A19 |
| | inhibitor | |
| 404 | a chromene COX-2 | A20 |
| | inhibitor | |
| 405 | a chromene COX-2 | A21 |
| | inhibitor | |
| 406 | a chromene COX-2 | A22 |
| | inhibitor | |
| 407 | a chromene COX-2 | A23 |
| | inhibitor | |
| 408 | a chromene COX-2 | A24 |
| | inhibitor | |
| 409 | a chromene COX-2 | A25 |
| | inhibitor | |
| 410 | a chromene COX-2 | A26 |
| | inhibitor | |
| 411 | a chromene COX-2 | A27 |
| | inhibitor | |
| 412 | a chromene COX-2 | A28 |
| | inhibitor | |
| 413 | a chromene COX-2 | A29 |
| | inhibitor | |
| 414 | a chromene COX-2 | A30 |
| | inhibitor | |
| 415 | a chromene COX-2 | A31 |
| | inhibitor | |
| 416 | a chromene COX-2 | A32 |
| | inhibitor | |
| 417 | C68 | A1 |
| 418 | C68 | A2 |
| 419 | C68 | A3 |
| 420 | C68 | A4 |
| 421 | C68 | A5 |
| 422 | C68 | A6 |
| 423 | C68 | A7 |
| 424 | C68 | A8 |
| 425 | C68 | A9 |
| 426 | C68 | A10 |
| 427 | C68 | A11 |
| 428 | C68 | A12 |
| 429 | C68 | A13 |
| 430 | C68 | A14 |
| 431 | C68 | A15 |
| 432 | C68 | A16 |
| 433 | C68 | A17 |
| 434 | C68 | A18 |
| 435 | C68 | A19 |
| 436 | C68 | A20 |
| 437 | C68 | A21 |
| 438 | C68 | A22 |
| 439 | C68 | A23 |
| 440 | C68 | A24 |
| 441 | C68 | A25 |
| 442 | C68 | A26 |
| 443 | C68 | A27 |
| 444 | C68 | A28 |
| 445 | C68 | A29 |
| 446 | C68 | A30 |
| 447 | C68 | A31 |
| 448 | C68 | A32 |

Biological Assays

### Evaluation of COX-1 and COX-2 Activity in Vitro

The COX-2 inhibiting agents of this invention exhibit inhibition in vitro of COX-2. The COX-2 inhibition activity of the compounds illustrated in the examples above are determined by the following methods. The COX-2 inhibition activity of the other COX-2 inhibitors of the present invention may also be determined by the following methods.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (107-108 pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidbnic acid (10 μM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C./room temperature by transferring 40 μl of reaction mix into 160 μl ELISA buffer and 25 μM indomethacin. The PGE2 formed is measured by standard ELISA technology (Cayman Chemical).

### Biological Evaluation

A combination therapy of a COX-2 inhibiting agent and an aromatase inhibitor for the treatment or prevention of a neoplasia disorder or osteoporosis in a mammal can be evaluated as described in the following tests.

Lewis Lung Model

Mice are injected subcutaneously in the left paw (1×10⁶ tumor cells suspended in 30% Matrigel) and tumor volume is evaluated using a phlethysmometer twice a week for 30-60 days. Blood is drawn twice during the experiment in a 24 h protocol to assess plasma concentration and total exposure by AUC analysis. The data is expressed as the mean+/−SEM. Student's and Mann-Whitney tests are used to assess differences between means using the InStat software package. A COX-2 inhibitor and an aromatase inhibitor are administered to the animals in a range of doses. Analysis of lung metastasis is done in all the animals by counting metastasis in a stereomicroscope and by histochemical analysis of consecutive lung sections.

HT-29 Model

Mice are injected subcutaneously in the left paw (1×10⁶ tumor cells suspended in 30% Matrigel) and tumor volume is evaluated using a phlethysmometer twice a week for 30-60 days. Implantation of human colon cancer cells (HT-29) into nude mice produces tumors that reach 0.6-2 ml between 30-50 days. Blood is drawn twice during the experiment in a 24 h protocol to assess plasma concentration and total exposure by AUC analysis. The data is expressed as the mean +/− SEM. Student's and Mann-Whitney tests are used to assess differences between means using the InStat software package.

A. Mice injected with HT-29 cancer cells are treated with an aromatase inhibitor i.p at doses of 50 mg/kg on days 5,7 and 9 in the presence or absence of celecoxib in the diet. The efficacy of both agents is determined by measuring tumor volume.

B. In a second assay, mice injected with HT-29 cancer cells are treated with an aromatase inhibitor on days 12 through 15. Mice injected with HT-29 cancer cells are treated with an aromatase inhibitor i.p at doses of 50 mg/kg on days 12, 13, 14, and 15 in the presence or absence of celecoxib in the diet. The efficacy of both agents is determined by measuring tumor volume.

C. In a third assay, mice injected with HT-29 colon cancer cells are treated with an aromatase inhibitor i.p 50 mg/kg on days 14 through 17 in the presence or absence of celecoxib (1600 ppm) and valdecoxib (160 ppm) in the diet. The efficacy of both agents is determined by measuring tumor volume.

### NFSA Tumor Model

The NFSA sarcoma is a nonimmunogenic and prostaglandin producing tumor that spontaneously developed in C3Hf/Kam mice. It exhibits an increased radioresponse if indomethacin is given prior to tumor irradiation. The NFSA tumor is relatively radioresistant and is strongly infiltrated by inflammatory mononuclear cells, primarily macrophages which secrete factors that stimulate tumor cell proliferation. Furthermore, this tumor produces a number of prostaglandins, including prostaglandin E₂ and prostaglandin I₂.

Solitary tumors are generated in the right hind legs of mice by the injection of 3×10⁵ viable NFSA tumor cells. Treatment with a COX-2 inhibiting agent (6 mg/kg body weight) and an aromatase inhibitor or vehicle (0.05% Tween 20 and 0.95% polyethylene glycol) given in the drinking water is started when tumors are approximately 6 mm in diameter and the treatment ia continued for 10 consecutive days. Water bottles are changed every 3 days. In some experiments, tumor irradiation is performed 3-8 days after initiation of the treatment. The end points of the treatment are tumor growth delay (days) and TCD₅₀ (tumor control dose 50, defined as the radiation dose yielding local tumor cure in 50% of irradiated mice 120 days after irradiation). To obtain tumor growth curves, three mutually orthogonal diameters of tumors are measured daily with a vernier caliper, and the mean values are calculated.

Local tumor irradiation with single y-ray doses of 30, 40, or 50 Gy is given when these tumors reach 8 mm in diameter. Irradiation to the tumor is delivered from a dual-source ¹³⁷Cs irradiator at a dose rate of 6.31 Gy/minute. During irradiation, unanesthetized mice are immobilized on a jig and the tumor is centered in a circular radiation field 3 cm in diameter. Regression and regrowth of tumors is followed at 1-3 day intervals until the tumor diameter reaches approximately 14 mm.

The magnitude of tumor growth delay as a function of radiation dose with or without treatment with a COX-2 inhibiting agent and an aromatase inhibitor is plotted to determine the enhancement of tumor response to radiation. This requires that tumor growth delay after radiation be expressed only as the absolute tumor growth delay, i.e., the time in days for tumors treated with radiation to grow from 8 to 12 mm in diameter minus the time in days for untreated tumors to reach the same size. It also requires that the effect of the combined COX-2 inhibiting agent and aromatase inhibitor plus-radiation treatment be expressed as the normalized tumor growth delay. Normalized tumor growth delay is defined as the time for tumors treated with both a COX-2 inhibiting agent and radiation to grow from 8 to 12 mm in diameter minus the time in days for tumors treated with a COX-2 inhibiting agent and an aromatase inhibitor alone to reach the same size.

Ovariectomized Rat Model: A Model of Post-Menopausal Osteoporosis

In women, estrogen deficiency during the menopause results in increased bone turnover leading to bone loss. Ovariectomy in rats produces estrogen deficiency and increased bone turnover leading to trabecular bone loss similar to that observed in post-menopausal women (Kalu, D. N., Bone and Mineral 1991; 15:175; Frost, H. M., Jee W. S. S., Bone and Mineral 1992; 18:227; Wronski, T. J., Yen, C-F, Cells Materials 1991; (suppl. 1):69). The OVX rat is thus an appropriate model to evaluate compounds for the prevention and treatment of post-menopausal osteoporosis. The ability of bone resorption inhibiting COX-2 inhibitors and aromatase inhibitors in combination to inhibit estrogen deficiency bone loss is assessed in OVX rats, since ovariectomy causes significant bone loss in the lumbar vertebrae, proximal tibia, and distal femoral metaphyses (Ke, H. Z., et al., Endocrin 1995; 136:2435; Chen, H. K., et al., J Bone Miner Res 1995; 10:1256).

Seventy-five day old female Sprague Dawley rats (weight range of 225 to 275 g) are obtained from Charles River Laboratories (Portage, Mich.). They are housed in groups of 3 and have ad libitum access to food (calcium content approximately 1%) and water. Room temperature is maintained at 22.2° C. +/−1.7° C. with a minimum relative humidity of 40%. The photoperiod in the room is 12 hours light and 12 hours dark. One week after arrival, the rats undergo bilateral ovariectomy under anesthesia (44 mg/kg Ketamine TM and 5 mg/kg Xylazine TM (Butler, Indianapolis, Ind.) is administered intramuscularly). Treatment with vehicle or the test compositions is initiated either on the day of surgery following recovery from anesthesia or 35 days following the surgery. The rats are treated either with vehicle containing a bone resorption inhibiting combination of a COX-2 inhibitor and an aromatase inhibitor or with vehicle only. Oral dosage is by gavage in 0.5 mL of pH-adjusted 1% carboxymethylcellulose (CMC). Body weight is determined at the time of surgery and weekly during the study, and the dosage is adjusted with changes in body weight. Vehicle-treated ovariectomized (OVX) rats and non-ovariectomized (intact) rats are evaluated in parallel with each experimental group to serve as negative and positive controls. The rats are treated daily for 35 days (6 rats per treatment group) and are sacrificed by decapitation on the 36th day. The 35-day time period is sufficient to allow maximal reduction in bone density, measured as described below. At the time of sacrifice, the uteri are removed, are dissected free of extraneous tissue, and the fluid contents are expelled before determination of wet weight in order to confirm estrogen deficiency associated with complete ovariectomy. Uterine weight is routinely reduced about 75% in response to ovariectomy. The uteri are then placed in 10% neutral buffered formalin to allow for subsequent histological analysis.

Calcein at 10 mg/kg is injected s.c. into all rats 12 and 2 days before necropsy as a fluorochrome bone marker to measure bone dynamic histomorphometric parameters. The effects of a combination of COX-2 inhibitor and aromatase inhibitor on the following end points are determined: (a) serum osteocalcin, a biochemical marker of bone turnover, (b) bone mineral density of lumbar vertebrae and distal femoral metaphyses, (c) bone histomorphometry of fifth lumbar vertebral body and proximal tibial metaphyses.

For the measurement of the endpoints, serum osteocalcin concentration is determined by radioimmunoassay assays known in the art, and bone mineral content (BMC) and bone mineral density (BMD) are measured by standard procedures as described below:

The first to the sixth lumbar vertebrae from each rat are removed during necropsy. These are then scanned ex vivo using dual-energy X-ray absorptiometry. The scan images are analyzed, and bone area, BMC, and BMD of whole lumbar vertebrae (WLV), and LV1 through LV6 is determined.

Using dual-energy X-ray absorptiometry, the right femur of each rat is scanned ex vivo. Bone mineral density (BMD) of the distal femoral metaphyses (second 0.5 cm from the distal end of femur) and the proximal femur (the first 0.5 cm from the proximal end of femur, which contains the femoral head, neck, and greater trochanter) is determined. In order to determine the effects of a COX-2 inhibitor and an aromatase inhibitor on long bone metaphyses, histomorphometric analyses are performed on the proximal tibiae.

### Example 5

### Effect of Exemestane and Celecoxib Alone or in Combination on DMBA-Induced Mammary Carcinoma in Rats

The chemotherapeutic potential of exemestane (EXE) and celecoxib (CXB) alone and in combination was evaluated in DMBA-induced rat mammary tumors.

Tumor bearing rats were treated for four weeks, starting when tumor diameter was 1 cm. Doses of EXE and CXB yielding a limited response rate were used to highlight a potential synergistic activity. Experimental groups tested were: EXE 50 mg/kg/wk i.m. for four weeks, CXB in the diet (500 mg/kg of diet) for four weeks, the combination of these, vehicle alone, or ovariectomy. The test results are summarized in Table 12 below.

TABLE NO. 12
Combination And Solo Administration of
Exemestane and Celecoxib

| Treatment | | | | | |
|---|---|---|---|---|---|
| No. Rats/ | | | | Rats with | # |
| No. tumors | CR + PR, % | NC, % | P, % | NT, % | NT per rat |
| Vehicle | 0 | 5 | 95 | 73 | 2.5 |
| (15/21) | | | | | |
| CXB (15/23) | 0 | 30 | 70 | 67 | 1.6 |
| Exe (15/23) | 5 | 78 | 17 | 67 | 0.9 |
| EXE + CXB | 48 | 47 | 5 | 47 | 0.6 |
| (15/23) | | | | | |
| Ovariectomy | 96 | 4 | 0 | 0 | 0 |
| 15/26 | | | | | |

CR, complete response;
PR, partial response;
NC, no change;
P, progression;
NT, new tumors appearing during the 4-week study period

As demonstrated the combination of EXE and CXB is significantly more effective than either alone in reducing tumor growth and in reducing new tumor incidence in a hormone-dependent breast cancer model.

The contents of each of the references cited herein, including the contents of the references cited within these primary references, are herein incorporated by reference in their entirety.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the methods, combinations and compositions of the present invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A composition comprising an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention, or inhibition of a disorder selected from the group consisting of a neoplasia, a neoplasia-related disorder, and osteoporosis.

2. The composition of claim 1 wherein the source of the COX-2 inhibitor is a COX-2 inhibitor.

3. The composition of claim 2 wherein the COX-2 inhibitor is a COX-2 selective inhibitor.

4. The composition of claim 1 wherein the source of the COX-2 inhibitor is selected from the group consisting of celecoxib, deracoxib, valdecoxib, rofecoxib, etoricoxib, meloxicam, and parecoxib.

5. The composition of claim 4 wherein the COX-2 selective inhibitor is celecoxib.

6. The composition of claim 4 wherein the COX-2 selective inhibitor is deracoxib.

7. The composition of claim 4 wherein the COX-2 selective inhibitor is valdecoxib.

8. The composition of claim 4 wherein the COX-2 selective inhibitor is rofecoxib.

9. The composition of claim 4 wherein the COX-2 selective inhibitor is etoricoxib.

10. The composition of claim 4 wherein the COX-2 selective inhibitor is meloxicam.

11. The composition of claim 3 wherein the COX-2 selective inhibitor is a compound of Formula (4) 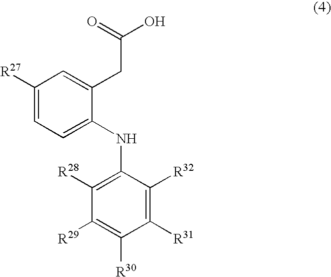
or an isomer, pharmaceutically acceptable salt prodrug or ester thereof, wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxy;
R³¹ is hydrogen, fluoro, or methyl; and
R³² is chloro, fluoro, trifluoromethyl, methyl, or ethyl,
provided that R²⁸ R²⁹ R³¹ and R³² are not all fluoro when R²⁷ is ethyl and R³⁰ is H.

12. The composition of claim 11 wherein:
R²⁷ is propyl;
R²⁸ and R³⁰ are chloro;
R²⁹ and R³¹ are methyl; and
R³² is ethyl.

13. The composition of claim 11 wherein:
R²⁷ is methyl;
R²⁸ is fluoro;
R³² is chloro; and
R²⁹, R³⁰ and R³¹ are hydrogen.

14. The composition of claim 1 wherein the aromatase inhibitor is selected from the group consisting of
aminoglutethimide;
anastrozole;
atamestane;
4,4′-(2H-tetrazol-2-ylmethylene)-bisbenzonitrile;
4,4′-(fluoro-1H-1,2,4-triazol-1-ylmethylene)-bisbenzonitrile;
exemestane;
fadrozole;
4-amino-6-methylene-androsta-1,4-diene-3,17-dione;
finrozole;
formestane;
4-[1-(2-hydroxyphenyl)-2-(1H-imidazol-1-yl)ethenyl]benzonitrile;
letrozole;
liarozole;
4-(2-benzofuranyl-1H-1,2,4-triazol-1-ylmethyl)benzonitrile;
N-[(2-chlorophenyl)methyl]-6-(1H-imidazol-1-yl)-3-pyridazinamine dihydrochloride;
minamestane;
(7Z)-6-(4-chlorophenyl)-6,7-dihydro-7-(4-pyridinylmethylene)-8(5H)-indolizinone;
14-hydroxy-androst-4-ene-3,6, 17-trione;
1-[[(2S,3aR)-3a-ethyl-9-(ethylthio)-2,3,3a,4,5,6-hexahydro-1H-phenalen-2-yl]methyl]-1H-imidazole monohydrochloride;
pentrozole;
rogletimide,
10-[2-(methylthio)ethyl]-estra-4,9(11)-diene-3, 17-dione;
10-[2-(methylthio)ethyl]-estr-9(11)-ene-3,17-dione;
2-(1H-imidazol-1-yl)-4,6-di-4-morpholinyl-1,3,5-triazine;
N-(3-hydroxy-14-methyl-1-oxopentadecyl)-□-glutamylomithyl-tyrosylthreonyl-□]-glutamylalanylprolyl-glutam inyltyrosyl-(10□3)-lactone;
4-(2,6-dihydroxybenzoyl)-3-formyl-5-hydroxy-benzoic acid;
testolactone;
(4aS,4bR,5R,-10aR,10bS,12aS)-1,3,4,4a,4b,5,6,10a,10b,11,12,12a-dodecahydro-5-mercapto-10a, 12a-dimethyl-8H-phenanthro[2,1-c]pyran-8-one;
(4aS,4bR,5R,-10aR,10bS,12aS)-3,4,4a,5,6,10a,-10b,11,12,12a-decahydro-5-mercapto-10a,12a-dimethyl-1H-phenanthro[2,1-c]pyran-1,8(4bH)-dione;
vorozole;
4-[[(4-bromophenyl)methyl]-4H-1,2,4-triazol-4-ylamino]benzonitrile; and
4-[[(3,5-difluorophenyl)methyl]-5-pyrimidinylamino]benzonitrile.

15. The composition of claim 14 wherein the aromatase inhibitor is selected from the group consisting of
aminoglutethimide;
anastrozole;
atamestane;
exemestane;
fadrozole;
finrozole;
formestane;
letrozole;
testolactone; and
4-[[(4-bromophenyl)methyl]-4H-1,2,4-triazol-4-ylamino]benzonitrile.

16. The composition of claim 15 wherein the aromatase inhibitor is aminoglutethimide.

17 The composition of claim 15 wherein the aromataseinhibitor is anastrozole.

18. The composition of claim 15 wherein the aromatase inhibitor is atamestane.

19. The composition of claim 15 wherein the aromatase inhibitor is exemestane.

20. The composition of claim 15 wherein the aromatase inhibitor is fadrozole.

21. The composition of claim 15 wherein the aromatase inhibitor is finrozole.

22. The composition of claim 15 wherein the aromatase inhibitor is formestane.

23. The composition of claim 15 wherein the aromatase inhibitor is letrozole.

24. The composition of claim 15 wherein the aromatase inhibitor is testolactone.

25. The composition of claim 15 wherein the aromatase inhibitor is 4-[[(4-bromophenyl)methyl]-4H-1,2,4-triazol-4-ylamino]benzonitrile.

26. The composition of claim 1 wherein the disorder is a neoplasia or a neoplasia-related disorder.

27. The composition of claim 26 wherein the neoplasia or the neoplasia-related disorder is selected from the group consisting of a malignant tumor growth, benign tumor growth and metastasis.

28. The composition of claim 27 wherein the neoplasia or the neoplasia-related disorder is a malignant tumor growth selected from the group consisting of acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, esophageal cancer, Ewing's sarcoma, eye and orbit cancer, female genital cancer, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, stomach cancer, striated muscle cancer, submesothelial cancer, superficial spreading melanoma, T cell leukemia, testicular cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, and Wilms tumor.

29. The composition of claim 27 wherein the neoplasia or the neoplasia-related disorder is a benign tumor growth selected from the group consisting of a cyst, polyp, fibroid tumor, endometriosis, benign prostatic hypertrophy and prostatic intraepithelial neoplasia.

30. The composition of claim 27 wherein the neoplasia or the neoplasia-related disorder is metastasis.

31. The composition of claim 1 wherein the disorder is osteoporosis.

32. A combination therapy method for the treatment, prevention, or inhibition of a neoplasia, a neoplasia-related disorder, or osteoporosis in a mammal in need thereof, comprising administering to the mammal an amount of a COX-2 inhibitor compound source and an amount of an aromatase inhibitor wherein the amount of the COX-2 inhibitor compound source and the amount of the aromatase inhibitor together comprise a therapeutically effective amount for the treatment, prevention, or inhibition of a disorder selected from the group consisting of a neoplasia, a neoplasia-related disorder, and osteoporosis.

33. The method of claim 32 wherein the source of the COX-2 inhibitor is a COX-2 inhibitor.

34. The method of claim 33 wherein the COX-2 inhibitor is a COX-2 selective inhibitor.

35. The method of claim 32 wherein the source of the COX-2 inhibitor is selected from the group consisting of celecoxib, deracoxib, valdecoxib, rofecoxib, etoricoxib, meloxicam, and parecoxib.

36 The method of claim 35 wherein the source of the COX-2 inhibitor is celecoxib.

37. The method of claim 35 wherein the source of the COX-2 inhibitor is deracoxib.

38. The method of claim 35 wherein the source of the COX-2 inhibitor is valdecoxib.

39. The method of claim 35 wherein the source of the COX-2 inhibitor is rofecoxib.

40. The method of claim 35 wherein the source of the COX-2 nhibitor is etoricoxib.

41 The method of claim 35 wherein the source of the COX-2 inhibitor is meloxicam.

42. The method of claim 34 wherein the COX-2 selective inhibitor is a compound of Formula (4) 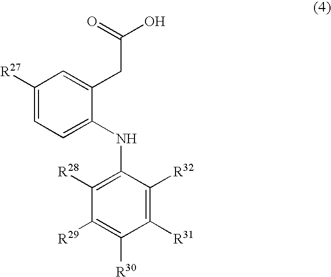
or an isomer, pharmaceutically acceptable salt prodrug or ester thereof, wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxy;
R³¹ is hydrogen, fluoro, or methyl; and
R³² is chloro, fluoro, trifluoromethyl, methyl, or ethyl,
provided that R²⁸, R²⁹, R³¹ and R³² are not all fluoro when R²⁷ is ethyl and R³⁰ is H.

43. The method of claim 42 wherein:
R²⁷ is propyl;
R²⁸ and R³⁰ are chloro;
R²⁹ and R³¹ are methyl; and
R³² is ethyl.

44. The method of claim 42 wherein:
R²⁷ is methyl;
R²⁸ is fluoro;
R³² is chloro; and
R²⁹, R³⁰ and R³¹ are hydrogen.

45. The method of claim 32 wherein the aromatase inhibitor is selected from the group consisting of
aminoglutethimide;
anastrozole;
atamestane;
4,4′-(2H-tetrazol-2-ylmethylene)-bisbenzonitrile;
4,4′-(fluoro-1H-1,2,4-triazol-1-ylmethylene)-bisbenzonitrile;
exemestane;
fadrozole;
4-amino-6-methylene-and rosta-1,4-diene-3,17-dione;
finrozole;
formestane;
4-[1-(2-hydroxyphenyl)-2-(1H-imidazol-1-yl)ethenyl]benzonitrile;
letrozole;
liarozole;
4-(2-benzofuranyl-1H-1,2,4-triazol-1-ylmethyl)benzonitrile;
N-[(2-chlorophenyl)methyl]-6-(1H-imidazol-1-yl)-3-pyridazinamine dihydrochloride;
minamestane;
(7Z)-6-(4-chlorophenyl)-6,7-dihydro-7-(4-pyridinylmethylene)-8(5H)-indolizinone;
14-hydroxy-and rost-4-ene-3,6,17-trione;
1-[[(2S,3aR)-3a-ethyl-9-(ethylthio)-2,3,3a,4,5,6-hexahydro-1H-phenalen-2-yl]methyl]-1H-imidazole monohydrochloride;
pentrozole;
rogletimide;
10-[2-(methylthio)ethyl]-estra-4,9(11)-diene-3,17-dione;
10-[2-(methylthio)ethyl]-estr-9(11)-ene-3,17-dione;
2-(1H-imidazol-1-yl)-4,6-di-4-morpholinyl-1,3,5-triazine;
N-(3-hydroxy-14-methyl-1-oxopentadecyl)-□-glutamylomithyl-tyrosylthreonyl-□-glutamylalanylprolyl-glutaminyltyrosyl-(10□3)-lactone;
4-(2,6-dihydroxybenzoyl)-3-formyl-5-hydroxy-benzoic acid;
testolactone;
(4aS,4bR,5R,-10aR,10bS,12aS)-1,3,4,4a,4b,5,6,10a,10b,11,12,12a-dodecahydro-5-mercapto-10a,12a-dimethyl-8H-phenanthro[2,1-c]pyran-8-one;
(4aS,4bR,5R,-10aR,10bS,12aS)-3,4,4a,5,6,10a,-10b,11,12,12a-decahydro-5-mercapto-10a,12a-dimethyl-1H-phenanthro[2,1-c]pyran-1,8(4bH)-dione;
vorozole;
4-[[(4-bromophenyl)methyl]-4H-1,2,4-triazol-4-ylamino]benzonitrile; and
4-[[(3,5-difluorophenyl)methyl]-5-pyrimidinylamino]benzonitrile.

46. The method of claim 45 wherein the aromatase inhibitor is selected from the group consisting of
aminoglutethimide;
anastrozole;
atamestane;
exemestane;
fadrozole;
finrozole;
formestane;
letrozole;
testolactone; and
4-[[(4-bromophenyl)methyl]-4H-1,2,4-triazol-4-ylamino]benzonitrile.

47. The method of claim 46 wherein the aromatase inhibitor is aminoglutethimide.

48. The method of claim 46 wherein the aromatase inhibitor is anastrozole.

49 The method of claim 46 wherein the aromatase inhibitor is atamestane.

50. The method of claim 46 wherein the aromatase inhibitor is exemestane.

51. The method of claim 46 wherein the aromatase inhibitor is fadrozole.

52. The method of claim 46 wherein the aromatase inhibitor is finrozole.

53. The method of claim 46 wherein the aromatase inhibitor is formestane.

54. The method of claim 46 wherein the aromatase inhibitor is letrozole.

55. The method of claim 46 wherein the aromatase inhibitor is testolactone.

56. The method of claim 46 wherein the aromatase inhibitor is 4-[[(4-bromophenyl)methyl]-4H-1,2,4-triazol-4-ylamino]benzonitrile.

57. The method of claim 32 wherein the disorder is a neoplasia or a neoplasia-related disorder.

58. The method of claim 57 wherein the neoplasia or the neoplasia-related disorder is selected from the group consisting of a malignant tumor growth, benign tumor growth and metastasis.

59. The method of claim 58 wherein the neoplasia or the neoplasia-related disorder is a malignant tumor growth selected from the group consisting of acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, esophageal cancer, Ewing's sarcoma, eye and orbit cancer, female genital cancer, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, stomach cancer, striated muscle cancer, submesothelial cancer, superficial spreading melanoma, T cell leukemia, testicular cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, and Wilms tumor.

60. The method of claim 58 wherein the neoplasia or the neoplasia-related disorder is a benign tumor growth selected from the group consisting of a cyst, polyp, fibroid tumor, endometriosis, benign prostatic hypertrophy and prostatic intraepithelial neoplasia.

61. The method of claim 58 wherein the neoplasia or the neoplasia-related disorder is metastasis.

62. The method of claim 32 wherein the disorder is osteoporosis.

63. A pharmaceutical composition comprising an amount of a COX-2 inhibitor compound source and an amount of an aromatase inhibitor and a pharmaceutically-acceptable excipient.

64. A kit that is suitable for use in the treatment, prevention or inhibition of a neoplasia or a neoplasia-related disorder or osteoporosis, wherein the kit comprises a first dosage form comprising a COX-2 inhibitor compound source and a second dosage form comprising an aromatase inhibitor, in quantities which comprise a therapeutically effective amount of the compounds for the treatment, prevention or inhibition of a neoplasia or a neoplasia-related disorder or osteoporosis.
